SEQUENCE LISTING (29 sequences)

SEQ ID NO: 1 - protein, 935 aa, Triticum aestivum
MEAALVSAATGALKPVLEKLGALVGDKYKRFKGVRGEIKSLTHELATMDAFLLKMSDEEDPDAQDKAWMNEVRELSYDMEDSIDDFMQSVDAEDTKPDGFLEKMKNLLGKIKARRRIGNEVQDLKKQIIEVAQRNERYKAREAFSKKENATIDPRALAIFQHASELVGIDEPKAELIKLLTQGESTRDKMNLVSIVGSGGMGKTTLANQVYQDLKGKFKCRVFLSVSRNPDMMNIFRTILSQVSGQRYGDTEAGSIQQLIIKIAEFLLDKRYFVVLDDIWDVVTWHTIKHAFPMRSSGSIIITTTRINEVAKSCCSTPFSGDIYCIRPLDMVHSRKLFYTRLFNSEENCPSHLKKVSEDILKKCDGLPLAIIAISGLLANTERTEGPWKQVKDSIGRALQRNTIVQEMMKILSLSYFDLPAHLKSCLLCLSIFPEDSVIKKKVLIKRWIAERLIHTEAGYSITYEFGERCFNELVNRSLIQPGETNRYGMVKYCRVHDTILDFIISKSIEENFVTLVGVPGLTVGTQSKVRRLSLQASKQKELIVPKGLVLSHVRSLDVFGISVKIPSMDKFRHLRFLDFEDCDQLENHHLENIGKLFQLRYLSLLRAEKVSKLPEQIGHLWCLEILDLRDTSVCELPASIVNLKRLVRLLVNKNVTLPCGISKLQALEKLKIVSVYSQSFNFLQEFEQLQSLKALTLDFEDYSSADRVNAERESKKDIIVASLKNLGNLLSLTVWDGPELVGESLCPVPLSLRKLMAWYSSIPHVPKWVGSLVNLQELCLELVGAQKDFYILGGLPVLRYLVLRIVEREVPRVIVSGEVGFPCLRIFKYYIWYAGIHLSFAAGAMPKLEDLSIIFDATKTESLGTSGAFDLGIENLPSLVTLDCKARGDDESRAKAAKAALREAADAHPNHPSLKFSEVSVRWDRDPVMLERRY

SEQ ID NO: 2 - DNA, 2808 bp, Triticum aestivum
atggaggcggccctggtgagtgcagcgacgggagccttgaaacctgtcctggagaagctaggcgctctagtgggcgataagtacaagaggttcaagggtgtgcgtggggagatcaaatcgctcactcatgaactagccacgatggatgcatttcttctaaagatgtcggatgaggaggatcctgatgctcaggataaggcgtggatgaatgaggtgcgggagctctcctatgacatggaggactccatcgacgacttcatgcagagtgttgacgccgaagatacaaagccagatggcttcttggagaagatgaagaacttgttggggaagataaaggctcggcgccggatcggcaacgaggtacaagatctaaagaagcaaattattgaggtggcccagaggaatgaaaggtacaaggcccgtgaggccttctccaagaaagaaaatgcaaccatcgaccccagagctcttgccatctttcagcatgcctccgagcttgtcggaattgatgaacccaaggctgagctaattaaactgttgacacaaggtgagtcaacacgagacaaaatgaacttggtatccattgttggatctgggggaatgggaaagacaactcttgcaaaccaagtgtatcaagatctcaaaggaaaattcaaatgtcgggtcttcctatcagtgtctaggaatccagacatgatgaatatctttaggactattcttagtcaagttagtggccaacgttacggtgacactgaagcaggaagtatacaacaactcatcatcaagattgccgaattcctattagacaaaagatattttgttgttcttgacgatatttgggatgtggttacatggcatactattaagcatgcatttcctatgagaagttctggcagtatcataatcaccactacccgtataaatgaagttgctaaatcatgttgttcaacaccattcagtggagatatttattgcataaggcctcttgatatggtgcactctaggaagttattttacacaagactattcaactcagaagaaaattgcccctcacaccttaaaaaagtttctgaggatattttgaaaaaatgtgatgggttgccattggcaatcattgctatatctggtttgttggctaatactgaaaggacagaggggccatggaaacaagtcaaagattcaattggtcgagcacttcaaagaaataccattgttcaagaaatgatgaagatattgtcacttagttactttgatctgcctgctcatcttaaatcttgtcttttatgtttgagtatattccctgaagattctgttatcaagaagaaagttttgataaagaggtggattgctgaaagattaattcatacagaagccggatatagtataacatatgagtttggtgaaaggtgttttaatgagctcgtcaataggagtttgatccaacctggggaaaccaacagatatggtatggtaaagtattgtcgagttcatgacacaattcttgatttcatcatatccaaatccatcgaagaaaactttgttactttggtaggtgttcctggtttgactgttggcacacaaagcaaagttcgtcggctgtccctgcaagccagcaagcaaaaagaattgatagtgccaaaaggcttggtgttgtctcatgtccgatcacttgatgtgttcgggatatcagtgaaaatcccttctatggataagttcaggcatttgcgttttctggactttgaagattgcgaccaactggagaaccatcatcttgaaaatatagggaagttgtttcagctgaggtacctgagcctcctaagagcggagaaagtaagtaagctcccagaacagataggacacctatggtgcttagagatactggatttaagagacacctctgtttgtgagttaccagcatctattgtcaatctcaagagattggtgcgtctattagtaaataagaatgttacacttccttgtggaatttccaagctgcaagcactggagaaattgaagattgtgagtgtctatagccagtcatttaacttcctgcaagaatttgagcagctgcagagtctgaaggcattgacccttgattttgaggattatagttctgctgatcgagtgaacgctgaaagagagtccaagaaagatattattgtcgcttcccttaaaaacctagggaaccttctctctctaactgtttgggatggccccgaattagtaggggaatctctgtgccctgtgccacttagcctccggaagcttatggcctggtattcgagtattccccatgttccaaagtgggtgggatcccttgtcaacctccaggagttatgccttgaattggtcggagctcagaaagatttctatatccttggaggcttacctgttctgcgttatctggttctgcgaattgttgaaagggaagttccgagggtcatagtctctggcgaagttggattcccatgcttgaggatatttaaatattatatttggtatgctgggatccatttgtcgtttgcggctggagccatgccaaagttagaagacctctcgataatatttgacgcaacaaaaaccgagtctctcggtactagcggtgcttttgatctcggaatcgaaaatctccccagcctcgttaccctcgactgtaaagcgcggggagacgatgaaagcagagctaaggctgcaaaggctgcccttcgggaagcagcggacgcacatcccaaccaccctagcctaaagttttcagaggtctccgttagatgggacagggatccagtgatgctggaacggaggtactaa

SEQ ID NO: 3 - protein, 948 aa, Triticum turgidum subsp. durum
MEAALVTVATGVLKPVLGKLATLLGDEYKRFKGVRKEIRSLTHELAAMEAFLLKMSEEEEDLNVQDKVWMNEVRELSYDMEDAIDDFMQSVGDKEEKPDGFIDKIKSSLGKLGNMKARHRIGKEIQDLKKQIIEVGDRNARYKGREIFSKAVNVTVDPRALAIFEHASKLVGIDEPKAELIKLLTDKDGVASTQQQVKMVSIVGSGGMGKTTLANQVYQELKEKFKCKAFISVSRNPDMTNILRTLLSEVGCQDYADTEAGSIQQLIRKITDYLAEKRYIIVIDDIWDVKTWDVIKCAFPMTRCGGVIITTTRLSDVARSCHSSIGGHIYNIRPLNMEHSRQLFHRRLFSSEEDCPSSLVKVSNQILEKCDGLPLAIIAIAGLLANTGRSEHLWNQVKDSIGRALERNPNVEVMIKILSLSYFDLPPHLKTCLLYLSIFPEDSIIEKKTLISRWIAEGFIQKEGIYTAYEVGVRCFNELINRSLIQPVKKDDYRGKSCRVHDIILDFIVSKSIEENFVTFAGVPSLTTVTQGKVRRLSMQVEGKGDSILPMSPILSHVRSFNVFRNRVNIHSTMEFRHLRVVDFNDSLLENHHLANVGRLLQLRYLSIYMTAVSELPEQIGHLQCLEMLDIRYTMVSELPASIVNLGKLAHLLLGSEDTCVKFPDGIAKMQALETLDEVDASKQSYNFLQGLGRLKNLRKLHIDYHDVAQEDKEVIASSLGKLCTQNLCSLTMRGNDDDDFLLNTWCTSPPLNLRKLVIWGCIFPKVPHWVGSLVNLQKLRLHVGKEIRHEDICILGALPALLTLGLKGMQKQPSCEDGRLAVSGEAGFRCLRKFKYCRWGDRMDLMFTAKCMPKLEKLKIIFYRHAQDEAPIIPAFDFGIENLSRLTTFKCHLGCRPMATRTFDAVKASLDRVVRAHPNHLTVIFSYPLRTSDMTYTFHDCYMRSQD

SEQ ID NO: 4 - protein, 1624 aa, Triticum monococcum
MSGLLGTVVDATIGWLVQSILDSFFTGRMEAWTREIGLAEDVEKLKFQMRDVQMVLAAAEGRRIDNMPLALSLDGLRELLYDSEDVMDELDYYRLEQQITEGKGCSAPTGTNLEGSYVSSSTLSSVVKSVCSATSQITDWISRGRKRKREEEGPAHCNMLPFEIKDSISKRINVIVNLLCSSSNSVKGVLQLENLRTMATSSKSQNIARDHRMTTSVPIEYKVYGRDAERDEIIDLLIKGGSSDLNVLPVVGSGGIGKTTLVRYVYHAKRIKDHFDLLIWVCVSTNFDVVGLTLQILDHVCEDRPYEKKSSLNKLQEILQENIRNKRFLLVMDDMWEEKDRGGWIKLLAPLKSNKVKGCMVLATTRTKSVAKMIGTMDEITLTGLDEKEFWLFFKACAFRNDNREHHPSLQSIGKQIAKALKGFPLAAQSVGALLSTEVSYQHWTTVRDKWKSLQGYDDDILPILKLSYDYLPVYLQRCFSYCSLYPEDYGFDGKELVHAWISQNFVQCKDPTIRLEETGHEYLEKLVDLGFFQKDGSHYVMHDLMHALAGMVSSNECATIDGLKSGAIRASVRHLSIIITDYDKDEHVSNSSEKFDKILQKVSWHKLRTLMLFGRSSIHLSGPLRTLCNVAKCLRLLSVTGADISSIYNSSNLFHLRYISAYRVSNPAFRQALTRCYHLQVLDVGISGNLDVPTDMNNLVNLRHLIAHEKVHHAIDCVSNMTSLQELKFKVQNVGSFEIGQLQSMNELVSLGVSQLENVKTKEEAWAAMLTHKEYLETLFLSWENSSMSLQPEAAEDVLDGLQPHQNLKTLEITGYGGAISPTWLSSAFSVTSLQILHLEECREWQILSTHGMHSLRKLTLIRMLNLMELSVPSLVELILIGMPKLKKCTGSYGMDLTSHLSVLMIKNCPQLNELTLFQSYSSFDAEQKSWFPSLSKLSIGQCPHIINNWPILPLREMGALKELELMDLHVVRVSVPSLEKLVLTKMPILEYCSSLTTSPPLQSSPPEGDQNELVSNLRRLTIHDCPCLIVSHPLPPSALISELSISGVSTLPTMRINWRHFTIESNELCMLDESILAFHNLRGITLFQIRSCPNLISLSSESFSQLIALETLSIHECPNLTMSNIMPEVVQENSTSTSSLVPPSLKRVNISTCGVTGRWLSQLLSHSQSLEELLLTGCPQIKFLSISQPRETEGTSSLVSAVTTSAQDEHELKLPYNLLCSLKALWIQLSLDLEFCGGYRDFAGFTSLMDLVLFGCPKLVSSLVGETKDDGTMEVGLLPPALEKVTISPLPESLRSFTPQGLLHLKELSLFYGPCLKSVQLHSCTSLVELGITGCVQLAVLEGLQFLTSLRSLDIEMSPELSSAWDLKLQEQEQGGNQIQLLPPSLDKLEIRALTDSVQSHLLSCLPAITKLAIGRSPELTSLQLGCCTALKELEIEKCDSLESIEGLQFCRNLKSLRVFNSPGLASFLELVSHQQGASVTLSGLQTLEASDGSVLSTHFCKQLTSLRLLQFGSEASEGRGESLVSLTEEQEGALQLLTSLQELQFRRCQNLLSLPANLNSLTSLETLSIIHCKSITRLPDMGLPTSLRYLKLFYCSEELAVQCRIVATEKLRVRIDRQDVN

SEQ ID NO: 5 - protein, 941 aa, Triticum aestivum
MAEVLVSASTGAMGSLLRKLGAMLTDEYKLLKNVRGDIKFLKDELEVMCAFLLKMSDVEEPDEPTKLRVTAVREMSYKIEDNIDKFMVLVEQEHGSSCSEAAHGVAKLMDKCKNLLPDIKARRRIAKEVKDIKKEIKDVSDRFSRYKIDDSSSSMPAKDKVDPRLRAVYKDAAELVGIDGPKDELVKWLNEKEGQSLKSVSIVGYGGLGKTTLANQIRVNLGATFDCGAFVSISRKPDMKAILRSILSQITKKDDACSRLDDIQLIIDKIREFLQDTRYFIIIDDIWELGTWETLKCAFVKNTLGSRIIITTRIVDVAKSCSPSSEDLVYEMKPLSEADSKKLFFKRIFGCEESCPDSLKEAANDILKKCRGLPLAINAISSVLVTTRETKEEWDRVRHSIRSSKVKSDIIETMNYILSLSYFDLPHHLRSCLLYLALFPEDQLIGRKRLVRRWISEGFIHGESGQDLMELGEEYFHQLVNRSLIQPGNIGYDGKAMYCRVHDTILDFLIDKSSEENMCTVLKKQCKPNGIVRRLSLMGNEDEEIVEQLDLSHARSITAFGDIKLLPSLGRSKCLRVLDLQDCDQLENHHIKDIERLYQLRYLDISSTGITELPRQIGELLYLETLVAYGLRELPESTSRLQRLARLFVYSGCKLPGGLGNLINLQELDCVDALHLKHVEELGKLTNLRKLSIKLDTGGIEGNKLEESKEKLVSSLCKLDECGLLSLSIDYYLREKDGEEPFLPALGCIQEVFVYGQDISRISRWLASLPNLHRLLLDDPKIEQQDIEMIGLIPNLIDLTLPPLYKTDDAGRLIIRREGFQQLQKFEAYNTRMGVLMFEPGAMPRLKELKLHNFIEKPKSAAVDFDFGIQRLSSLARLTVSLSCGGWTVAEVEAAEDAFKSMAEANPNRPILEMTRYNTQHMLQDEQIGMTGSATTPAVKS

SEQ ID NO: 6 - protein, 712 aa, Aegilops tauschii
MDIVTGAIAKLIPKLGELLVGEYKLHKGVKKNIEDLLKELKTMNAALIKIGEVPPDQLDSQDKLWADEVRELSYVIEDAVDKFLVRVHGVEPDDNTNGFKGLMKRTTKLLKKVVDKHGIAHAIKDIKKELQEVAARRDRNKFDGIASIPTEAIDPRLRALYIEAAELVGIYGKRDQELMSLLSLEGDDASTKKLKKVSIVGFGGLGKTTLAKAVYEKIKGDFDCHAFVPVGQNPDKKKVFRDILMDLSNSNSDLALLDERQLINKLHKFLENKRYLVIIDDVWDEGLWKDINLAFSNRNNLGSRLIITTRIFGVSESCCSSADDPVYEIEPLSIDDSSKLFYTRIFSDSGCPKEFEQVSKDILKKCGGVPLAIITIASALASGQQVKPKHEWDILLQSLGSGVTKDNSLVEMRRILSFSYYNLPSHLKTCLLYLCIYPEDSMIHRDRLIWKWVAEGFVHHGDQGTSLFLVGLNYFNQLINRSMLQPIYSDMGNVYACRVHDMVLDLICNLSHEAKFVNVFDGTGNIMSSQSNVRRLSLQNKNEDHQAKPLTNIMSISQVRSITIFPPAVSIMPALSRFEVLRVLDLSDCNLGESSSLQPNLKGVGHLIHLRYLGLSGTRISKLPAEIGTLQFLEVLDLGYNHELDELPSTLFKLRRLIYLNVSPYEVVPTPGVLQNMTSIEVLRGIFVSEHYCTRAWQTGKAEGASDLLQGW

SEQ ID NO: 7 - protein, 919 aa, Triticum monococcum subsp. monococcum
MEIAMGAIGSLLPKLGELLIGEITLEKKVRKGIESLITELKLMQAVLSKVSKVPADQLDEGVKIWAGNVKELSYQMEDIVDAFMVRVGDGGESTNPKNRVKKILKKVKKLFKNGKDLHRISAALEEVVLQAKQLAELRQRYEQEMRDTSANTSVDPRMMALYTDVTELVGIEETRDKLINMLTEGDDWSKHPLKTISIVGFGGLGKTTLAKAAYDKIKVQFDCGAFVSVSRNPEMKKVLKDILYGLDKVKYENIHNAARDEKYLIDDIIEFLNDKRYLIVIDDIWNEKAWELIKCAFSKKSPGSRLITTTRNVSVSEACCSSEDDIYRMEPLSNDVSRTLFCKRIFSQEEGCPQELLKVSEEILKKCGGVPLAIITIASLLANKGHIKAKDEWYALLSSIGHGLTKNRSLEQMKKILLFSYYDLPSYLKPCLLYLSIFPEDREIRRARLIWRWISEGFVYSEKQDISLYELGDSYFNELVNRSMIQPIGIDDEGKVKACRVHDMVLDLICSLSSEENFVTILDDPRRKMPNSESKVRRLSIQNSKIDVDTTRMEHMRSVTVFSDNVVGKVLDISRFKVLRVLDLEGCHVSDVGYVGNLLHLRYLGLKGTHVKDLPMEIGKLQFLLTLDLRGTKIEVLPWSVVQLRRLMCLYVDYGMKLPSGIGNLTFLEVLDDLGLSDVDLDFVKELGRLTKLRVLRLDFHGFDQSMGKALEESISNMYKLDSLDVFVNRGLINCLSEHWVPPPRLCRLAFPSKRSWFKTLPSWINPSSLPLLSYLDITLFEVRSEDIQLLGTLPALVYLEIWNYSVFEEAHEVEAPVLSSGAALFPCATECRFIGIGAVPSMFPQGAAPRLKRLWFTFPAKWISSENIGLGMRHLPSLQRVVVDVISEGASREEADEAEAALRAAAEDHPNRPILDIW

SEQ ID NO: 8 - protein, 1230 aa, Triticum aestivum
MAEFVVRPLVSTLMNTASSYLLDQYKVMDGMKEQRETLKRQLPAILDIIQDAEKKGASLPGVRAWLEALKKVAYEANDVFDEFKYEALRRDAKKKGHYKKLGFDIVSLFPAHNPIVFRYRMGKKLCRIVRKIEGLVREMNDFGFNQTQQAPPSKQWRNTDSIIIDSEKDIVSRSRNEEKKKIVDILIDQAGDRDLIVLPIVGMGGLGKTTFAQLVYNDPIIKEHFKLQRWCCVSDDFDVVKIANNICETNEIHREKALQNLQKEVSGKRYLIVLDDVWNEDADKWEKLKTCLKHGGKGSAILTTTRNVQVARIMKMCIADSHNLRNLDKVFLKEIFENRAFCLQKPKAAELSDVVDKIMDRCGGSPLAAKAFGSMLSNKTSMKEWTDILARSNTCNEGTKTFLVLKLSYDDLPSHLKQCFAFCAVFPKDYEIGVETLIQLWMAHDFIPLKEGDNLEKVGREIFDELTWRSFFQDVKRIPRREWWGELRPRTICKIHDLMHDIALSVMGKDCLTIVDRPNEKELLSTGPTRYLFSSYEYIGTLLDDYLKKHSPALQTLLYPYPYTSDSAPHLSKCNYLRALQLFSLRKLPLWPRHLQHLRYLDLSNNMLIEELPKEISILYNLQTLNLCNCRRLDQLPEDMKYMENLRHLYTNGCSSLKCMPPGLGQLTSLQTLTYFVVSSSPGCSTIRELQDLNLGGELELSRLQFATEVDAKACSLGNKEKLTHLSLKWGDDSSDELGHHRNVLDALKPHAVLEFLRIRSYRGTGFPAWVVSINFLQHLTELQLDGCTMCEEFPQFGQFKSLEVLVLKRLNKLQSLCNHSSSAIFPALKVLRLKKLEIFERWVATEGEELAFPQLENVKIKDCPKLAILPEAPKLKFIALKEEKAQLSLSIFKSRYMACLSGVGLSVRDTEAAPRTELDQDCEVSLSNLLLDGCNFLFCSTPLQPTVGVWKWFGQLVHLEIKSCDMLIYWPEEEFRCLVSLNSLSINSCSKLVGHTQGKGCRTRTQVRDQLLPNLKNLRIHHCGSLTELFVLPPSLTSIDMLDCNSIESILGQDDTELESILHFDTASSSEHFNDLTSTCLLEQSLSPRINPLPCLDYLRIVSCKKLRFVPVQLDALQFLCISDCNGLESLDCLGDLPSLEYLYLMGCKHLASVPGSLGSYSALQKLRIEYCPALNMKPLHGHLQQRLDSLELKDLSKAGSSHPNEGPKLWEPKSWKYMIPSLRKRESE

SEQ ID NO: 9 - protein, 956 aa, Secale cereale
MNIVTGAMGSLIPKLGELLMDEYKLHKRIKKDVEFLKKELESMHAALIKVGEVPRDQLDRQVKLWADEVRELSYNMEDVVDKFLVRVDGDGIQQPHDNSGRFKELKNKMIGLFKKGRNHHRIADAIKEIKEQLQEVAARRDRNKVAVPNPMEPITIDPCLRALYAEATELVGIYGKRDEELMRLLSMEGDDASNKRLKKVSIVGFGGLGKTTLARAVYDKIKGDFDCRAFVPVGQNPDMKKVLRDILIDLGNPHSDLAILDDKQLVKKLHDFLENKRYLVIIDDIWDEMLWEGINFAFSNRNNLGSRLITTTRNFDVSKSCCLSADDSIYKMKPLSTDDSRRLFHKRIFPDAGGCPSEFQQVSEDILKKCGGVPLAIITIASALASGQHVKPKHEWDILLQSLGSGVTKDNSLVEMRRILSFSYYNLPSHLKTCLLYLCIYPEDSTIGRDRLIWKWVAEGFVHHGDQGTSLFLVGLNYFNQLINRSMIQPIYDELGQVHACRVHDMVLDLICNFSHEAKFVNVLDGTGNSISSQSNVRRLSLQNKMEDHQAKPLTNIMSMSRVRSITIFPPAVSIMPSLSMFEVLRVLDLSNCDLGKSSSLQLNLKGVGHLIHLRYLDLQGTQISELPTEIGNLQFLEVLDLDNNYELDELPSTLFKLRRLIYLNVMLYKVVPTPGVLQNMTSIEVLRGVLVSLNIIAQELGNLTRLRELKICFKDGNLDSYKLFVKSLGNLHHIESLSISYNSKETSFELMDLLGERWVPPVHLREFVSWMPSQLSALRGWIKRDPSHLSNLSELILWPVKEVQQEDVEIIGGLLSLRRLWIKSTHQTQRLLVIRADGFRCMMDFELNCGSAAQIMFEPGALPRAEVLVFSLGVRVAQEDGNCGFDLGLQGNLLSLRHDVFVRIYCGGARVGEAKEAEAAVRHALEAHPNHPPIDIEMTPYIAEGARDDDLCEEN

SEQ ID NO: 10 - DNA, 7246 bp, Triticum aestivum
catgggcgcggaccacctgcgcctggtccaccaaagccgtggccacgcctccctcttctctatctctcctgcgccacgagaggacaggagagactgcggcggtgctgtcccgtcccatcgggctttaaagctgtcgtctcgtcctcatctggtagtactgtattattcaattattggcctccaagttggccgaaggtgcgctccaagaggcaaccttctctctgtacaagaccaagaggcagacgacgtcgagcgcacgcggagagtagagtcaaccttctctcacaaaggagcaaccaactgctgaaagctggatcggatcctcggaatcgttcccgtgaattgaagctaggagaccaagctccgtcaagttcgtcatccaccttcacaagggcatctccaggttagttcttcaagccagatcttgctagctagctagcttcagttggcatctcgtgatgcggcggcatgattaagcgttcattatagaggtaattccatgttttacctcctcttttcggatgaagagagcttgtggtgagcagatctagctacgtaccagaagaactgttgaccaacttggtactccctccgttccaaaattgatgacctatacaaagttgggtcatcaattttggaacggagggagtacctagcaacgagcatctgagcttaattaccaatttcgggataagaaaaacttgtctttagctatgcaagttgagcgcaaagattaattgtttaattacggccggcgatgtaactatgctaccttttccccgcaaaaaagctatcctagcttcgctaagctgtaagccgagagagtaccatcattcagtctagctactctgttttcgccttcatatgcacaaaactaactgacaaagcgtacgtctcaggtagcaccgctcgcaaccatggaggcggccctggtgagtgcagcgacgggagccttgaaacctgtcctggagaagctaggcgctctagtgggcgataagtacaagaggttcaagggtgtgcgtggggagatcaaatcgctcactcatgaactagccacgatggatgcatttcttctaaagatgtcggatgaggaggatcctgatgctcaggataaggcgtggatgaatgaggtgcgggagctctcctatgacatggaggactccatcgacgacttcatgcagagtgttgacgccgaagatacaaagccagatggcttcttggagaagatgaagaacttgttggggaagataaaggctcggcgccggatcggcaacgaggtacaagatctaaagaagcaaattattgaggtggcccagaggaatgaaaggtacaaggcccgtgaggccttctccaagaaagaaaatgcaaccatcgaccccagagctcttgccatctttcagcatgcctccgagcttgtcggaattgatgaacccaaggctgagctaattaaactgttgacacaaggtgagtcaacacgagacaaaatgaacttggtatccattgttggatctgggggaatgggaaagacaactcttgcaaaccaagtgtatcaagatctcaaaggaaaattcaaatgtcgggtcttcctatcagtgtctaggaatccagacatgatgaatatctttaggactattcttagtcaagttagtggccaacgttacggtgacactgaagcaggaagtatacaacaactcatcatcaagattgccgaattcctattagacaaaaggtacaatttcagttttaacttctcatccttgagtatatagttaataaatttatttaaatattacacatcaaaaccatactatgtttttgtataataaaattagttccatgtacttttttgtccaattcttttttctcattagttggtacaaaaagttatggtcacatgtttgtagtcatgttcatgttgatgccaagtaaataaaaatagagagagtagcaaaaaccacaacatatgacatagccttagagcaactataatagggtgcttatagtcgaaaaatacatggatgaaccctccctagtacatatgaacccacttcggaaaacatatttgtaaatgcgaaaacattctgaaaaaagattgcatgggtacatctacatgttctatgtgcgtgcgaaagtttcgtggaaaaacaactttttttcttgcctgtgcaaaaaagacgaaaaaattgtgttgtgaaacgctatttaggagcattgaaatttgcctttttttgcggagacaaaacaaaaaaacattttttcacaaaactttgtgcctcacagatatttttgcgaacatgtatgtgtgacattttgttttgaattttttcaaattttaaaacacgttgaaaatgtatttttgaaaagtgggatcatacgtacctgggttcaggacatgctgactctattgtctatattgtcggggcgatagaccccgggtagcctcatccggaccttaggacatttcaagacatcggggctagcaaagcccctcaggccttccggacgcacagccggttccttgggccggctaggaccaagtggccggcttccagaagacggcgcacccttagaaggcggctttggggggaggccggctcccagcagacggcccctcctcccctcggagtctgcattcacctgctccgactagacatggcgtggctacagtatgagctgacccagcggccacccacccgcctggcgcgggactgtagccatgcgacccctgacgcggccctcgtcagcaagggtcacgctaccgccaaaggcagtcagtacggcccgcaggcggcgggccctacctgtcagcgggctcccagaagacggcgcccccttagaagacgccttcaggggcgggccggcttccagctggcggcctttccctcccctcggagtccgcactcacccactacgacatgacggagcgtggctgcagtatgcgctgaccaagcggccaccaacccgcctgacgtgggactgtagccatacgactcctgacgcggcctccgtcatcaagtacagagctacagcaaaaggcaatcagcgtggcccgcaggcggcgggccctacctatcggccggattctcggcagccggcggggccctccagaggcgggccccagcagccggcggagaacccggcgaccctagccactgacgtccgggtcctacacccggccgtattaccattgtacccctggggggttggcctataaagccccccagggctcacccatgcaaagggttcagccttcattcccacactccaccatagctaggagaagagagcagccctgcccttcttcttcctctagcaacacagctcaaggagcaaccttgtatactcttgatcatagtcatcatgcggagaccccgcagagcaggattaggggtgttatctctccggagagccccgaacctgggtacgtctcgtgtgcgatcgagctcgtgctcactcccgcttccagggcccgacgacgtcctcttggctccacccatgataagccaccccctggcatctgacgtgagattaccacgacatatatggaggggatagacaatgaaaaatgaggagagtgagctctcctgcaagagcctacctttacacgttctggaagcaaaatataatactccctccgtctgagtttatttgtcgcttaaacggatatatctagtactgaaatacatttagatacatccatttaagcgacaactaattctggacagatgtagtatctaatagccaatatcatcatatgtactatgagcaagtataacaatagagacaattgctggctatatctcattagcatgtcatatatggttagtctaacaaaccacgtatataatagttagctataaaaataattgccccattaatatctaacctatcttactctgaatatttcttggagctcatactataactagcgataaatctacaatttgctacccttcttcctccgccaacttagcacaaatatgatgtgtaaacttctataggccgcttacatattttactatacttgctctaatagacatgcttttagggagtagatcaagtagctaacctcgacccattattctaacactaacaccactcacattttataatgcgcgaaaagttcattttcctgcctatattagttattgacttggctcacttctcttcgttaagttttttgggcttccgtgatatcctaaaccatttttcggagtactttatctcatgatcagttttggacaagccatgccatgcgcgtcatcttcttctaagctcccttcaattatcagacataccagatcctccatgcatccatatggacatgttctccttcctcttctctcccgccctttccactaaacactgctatacttgtctacggggatcggttgtcatgagatccccaaggcctcggccgctgtctcgacctcgtgcaatcgtgtgcatcactgacaatgtagtcgctcaaatgaaaaaaaaatagtttggagttaagtggtcccataaattgtgtagagggaatgcaaggcaaaaagttattttatagtagaaatgaacttttccatatagcagtatgtagcttttcttgatttaaaatcataggatgtttggatctacacctagctagactcttcttttaattccaggatcaccctgataaacatcccttgtagactagttaaatatgtgaattcatcataatgatttggtcaatctcttgttcagtcacccttgatattttctatggtcatggccctgtttaagctgttatgtatcaccgaattctcataatctgtggaactaattagcatgtactcttttctatggtcatggccctatttagcagttgtgcaagatttcttacttagaagtatatttttatgttgccactgtgaacatgtttatagatttatatatttgtctatcttcttgtacactctttatatcattaccacactgatatcagccatgtatgcttgtgcagatattttgttgttcttgacgatatttgggatgtggttacatggcatactattaagcatgcatttcctatgagaagttctggcagtatcataatcaccactacccgtataaatgaagttgctaaatcatgttgttcaacaccattcagtggagatatttattgcataaggcctcttgatatggtgcactctaggaagttattttacacaagactattcaactcagaagaaaattgcccctcacaccttaaaaaagtttctgaggatattttgaaaaaatgtgatgggttgccattggcaatcattgctatatctggtttgttggctaatactgaaaggacagaggggccatggaaacaagtcaaagattcaattggtcgagcacttcaaagaaataccattgttcaagaaatgatgaagatattgtcacttagttactttgatctgcctgctcatcttaaatcttgtcttttatgtttgagtatattccctgaagattctgttatcaagaagaaagttttgataaagaggtggattgctgaaagattaattcatacagaagccggatatagtataacatatgagtttggtgaaaggtgttttaatgagctcgtcaataggagtttgatccaacctggggaaaccaacagatatggtatggtaaagtattgtcgagttcatgacacaattcttgatttcatcatatccaaatccatcgaagaaaactttgttactttggtaggtgttcctggtttgactgttggcacacaaagcaaagttcgtcggctgtccctgcaagccagcaagcaaaaagaattgatagtgccaaaaggcttggtgttgtctcatgtccgatcacttgatgtgttcgggatatcagtgaaaatcccttctatggataagttcaggcatttgcgttttctggactttgaagattgcgaccaactggagaaccatcatcttgaaaatatagggaagttgtttcagctgaggtacctgagcctcctaagagcggagaaagtaagtaagctcccagaacagataggacacctatggtgcttagagatactggatttaagagacacctctgtttgtgagttaccagcatctattgtcaatctcaagagattggtgcgtctattagtaaataagaatgttacacttccttgtggaatttccaagctgcaagcactggagaaattgaagattgtgagtgtctatagccagtcatttaacttcctgcaagaatttgagcagctgcagagtctgaaggcattgacccttgattttgaggattatagttctgctgatcgagtgaacgctgaaagagagtccaagaaagatattattgtcgcttcccttaaaaacctagggaaccttctctctctaactgtttgggatggccccgaattagtaggggaatctctgtgccctgtgccacttagcctccggaagcttatggcctggtattcgagtattccccatgttccaaagtgggtgggatcccttgtcaacctccaggagttatgccttgaattggtcggagctcagaaagatttctatatccttggaggcttacctgttctgcgttatctggttctgcgaattgttgaaagggaagttccgagggtcatagtctctggcgaagttggattcccatgcttgaggatatttaaatattatatttggtatgctgggatccatttgtcgtttgcggctggagccatgccaaagttagaagacctctcgataatatttgacgcaacaaaaaccgagtctctcggtactagcggtgcttttgatctcggaatcgaaaatctccccagcctcgttaccctcgactgtaaagcgcggggagacgatgaaagcagagctaaggctgcaaaggctgcccttcgggaagcagcggacgcacatcccaaccaccctagcctaaagttttcagaggtctccgttagatgggacagggatccagtgatgctggaacggaggtactaaatttttctcccacaaaaggccatgtacttctttaattcacaagatttatgtatcgaaactgctatgtagacgatggatgtgtgcacgacactgggacgattggcagtccagccgctgatatgcgctggatagtatgcatggacggcttgatatgtgctgtcgtgcacaagtcgtccggacggcgtcgtgtgtatagcaccgctgtgtatgcatctccctcattgcctcatatcatcggatgtctgtctttttcagagataagc

SEQ ID NO: 11 - protein, 73 aa, Artificial Squence
  Fragment of Sr33
IASIPTEAIDPRLRALYIEAAELVGIYGKRDQELMSLLSLEGDDASTKKLKKVSIVGFGGLGKTTLAKAVYEK

SEQ ID NO: 12 - protein, 73 aa, Artificial Sequence
  Fragment of Sr50
PNPMEPITIDPCLRALYAEATELVGIYGKRDEELMRLLSMEGDDASNKRLKKVSIVGFGGLGKTTLARAVYDK

SEQ ID NO: 13 - protein, 71 aa, Artificial Sequence
  Fragment of Sr26
AFSKKENATIDPRALAIFQHASELVGIDEPKAELIKLLTQGESTRDKMNLVSIVGSGGMGKTTLANQVYQD

SEQ ID NO: 14 - protein, 70 aa, Artificial Sequence
  Fragment of Sr22
SSSSMPAKDKVDPRLRAVYKDAAELVGIDGPKDELVKWLNEKEGQSLKSVSIVGYGGLGKTTLANQIRVN

SEQ ID NO: 15 - protein, 71 aa, Artificial Sequence
  Fragment of Sr35
RDTSANTSVDPRMMALYTDVTELVGIEETRDKLINMLTEGDDWSKHPLKTISIVGFGGLGKTTLAKAAYDK

SEQ ID NO: 16 - protein, 67 aa, Artificial Sequence
  Fragment of Sr45
PSKQWRNTDSIIIDSEKDIVSRSRNEEKKKIVDILIDQAGDRDLIVLPIVGMGGLGKTTFAQLVYND

SEQ ID NO: 17 - protein, 74 aa, Artificial Sequence
  Fragment of Sr46
NNEDTSSLNYMIKKSQDLANISRSLDDEDLVGVDENRGKLEQWLGSDDVERSLITLTGMGGLGKTALASNVYRK

SEQ ID NO: 18 - protein, 9 aa, Artificial Sequence
  P-loop consensus
  MISC_FEATURE (2)..(2): Xaa = any amino acid
  MISC_FEATURE (3)..(3): Xaa = any amino acid
  MISC_FEATURE (5)..(5): Xaa = any amino acid
  MISC_FEATURE (7)..(7): Xaa = Ser or Thr
  misc_feature (8)..(8): Xaa can be any naturally occurring amino acid
GXXGXGKXT

SEQ ID NO: 19 - protein, 9 aa, Artificial Sequence
  Sr26 p-loop
GSGGMGKTT

SEQ ID NO: 20 - protein, 14 aa, Artificial Sequence
  Sr26 p-loop extended
VSIVGSGGMGKTTL

SEQ ID NO: 21 - protein, 4 aa, Artificial Sequence
  kinase 2 motif consensus
  MISC_FEATURE (3)..(3): Xaa = any amino acid
DDXW

SEQ ID NO: 22 - protein, 4 aa, Artificial Sequence
  Sr26 kinase 2 motif
DDIW

SEQ ID NO: 23 - protein, 13 aa, Artificial Sequence
  Sr26 kinase motif 2 extended
RYFVVLDDIWDVV

SEQ ID NO: 24 - protein, 9 aa, Artificial Sequence
  Sr26 kinase 3a motif extended
  MISC_FEATURE (2)..(2): Xaa - = any amino acid
  MISC_FEATURE (3)..(3): Xaa - = any amino acid
  MISC_FEATURE (4)..(4): Xaa - = any amino acid
  MISC_FEATURE (5)..(5): Xaa - = any amino acid
  MISC_FEATURE (6)..(6): Xaa - = any amino acid
  MISC_FEATURE (8)..(8): Xaa - = any amino acid
GXXXXXTXR

SEQ ID NO: 25 - protein, 9 aa, Artificial Sequence
  Sr26 kinase 3a motif
GSIIITTTR

SEQ ID NO: 26 - protein, 13 aa, Artificial Sequence
  Sr26 kinase 3a motif extended
GSIIITTTRINEV

SEQ ID NO: 27 - protein, 9 aa, Artificial Sequence
  LRR domain consensus
  MISC_FEATURE (1)..(1): Xaa = any amino acid
  MISC_FEATURE (2)..(2): Xaa = any amino acid
  MISC_FEATURE (4)..(4): Xaa = any amino acid
  MISC_FEATURE (6)..(6): Xaa = any amino acid
  MISC_FEATURE (7)..(7): Xaa = any amino acid
  MISC_FEATURE (8)..(8): Xaa = any amino acid
  MISC_FEATURE (9)..(9): Xaa = any amino acid
XXLXLXXXX

SEQ ID NO: 28 - DNA, 23 bp, Artificial Sequence
  Oligonucleotide primer
ggaatactcgaataccaggccat

SEQ ID NO: 29 - DNA, 26 bp, Artificial Sequence
  Oligonucleotide primer
ttgccactgtgaacatgtttatagat

(19) United States
(12) Patent Application Publication
(10) Pub. No.: US 2022/0042034 A1
(43) Pub. Date: Feb. 10, 2022

(54) Rust Resistance Gene

(71) Applicant: Commonwealth Scientific and Industrial Research Organisation, Acton, ACT (AU)

(72) Inventors: Jianping Zhang, Acton (AU); Evans Lagudah, Ngunnawal (AU)

(21) Appl. No.: 17/297,728

(22) PCT Filed: Apr. 12, 2019
(86) PCT No.: PCT/AU2019/050331

§ 371 Date: May 27, 2021

(30) Foreign Application Priority Data
Nov. 30, 2018 (AU) 2018904568

(51) Int. Cl. C12N 15/82; C07K 14/415

(52) U.S. Cl. CPC C12N 15/8282; C07K 14/415

(57) ABSTRACT

The present invention relates to a plant which has integrated into its genome an exogenous polynucleotide encoding a polypeptide which confers resistance to at least one strain of Puccinia graminis.

### FIELD OF THE INVENTION

The present invention relates to a plant which has integrated into its genome an exogenous polynucleotide encoding a polypeptide which confers resistance to at least one strain of Puccinia graminis.

### BACKGROUND OF THE INVENTION

Stem or black rust, caused by the fungal pathogen Puccinia graminis, has a long history of causing devastating destruction of cereal crops and was documented as early as Roman times. Yield losses in wheat due to stem rust Puccinia graminis f. sp. tritici (Pgt), have been reported from almost all major wheat growing regions worldwide. The successful utilization of the 1B/1R translocation origin resistance (R) gene Sr31 in many CIMMYT derivate semi-dwarf, high yield potential cultivars during the well-known “Green Revolution” initiated by Dr Norman Borlaug reduced the incidence of stem rust disease worldwide for almost 40 years.

That was until the emergence of Ug99, a stem rust pathotype first identified in Uganda in 1999 that was reported to be virulent to Sr31 (Pretorius et al., 2000). Since then, numerous efforts have been made seeking Ug99 resistance genes (Singh et al., 2015). As a result, eight seedling R genes and two triple rust APR genes, namely Sr22, Sr33, Sr35, Sr45, Sr50, Sr13b, Sr21, Sr46, Lr34/Yr18/Sr57 and Lr67/Yr46/Sr56, were successfully identified and cloned as effective R genes against the Ug99 lineage (Saintenac et al., 2013; Mago et al., 2015; Zhang et al., 2017; Chen et al., 2018; Periyannan et al., 2013; Steuernagel et al., 2016; Krattinger et al., 2011; Moore et al., 2015).

Recently, disease epidemics have been reported as a result of re-emergence of the stem rust pathotype “Digalu” (TKTTF) in the UK after 60 years that rendered 80% of the UK wheat cultivars susceptible. Furthermore, in 2017, Europe reportedly had the most severe stem rust disease outbreaks for more than 50 years, and the vulnerable hosts expanded from wheat to barley. The causal pathotype of the Sicilian epidemic was first thought to be race TTTTF, a pathotype that has been previously identified from Tanzania and Rwanda, but later on was reconfirmed as TTRTF (Lewis et al., 2018; Bhattacharya et al., 2017). The significant pathogenicity difference between these two pathotypes is that TTRTF is virulent on Sr7a, Sr13b, Sr37, Sr44 and most importantly, the newly cloned Ug99 resistant R genes Sr33 and Sr35. The TTRTF race is also reported to give a high infection type on adult plant carrying the cloned Secale cereale derived Ug99 effective R gene Sr50. A report in 2016 of a new pathotype arose in Georgia, the U.S., that is virulent to Sr22 making the situation more urgent and demonstrated that the threat is not only from the Ug99 lineage.

The history involving wheat R genes versus mutating rust pathogen populations shows a repetitive scenario. In some cases, even while the scientists were still celebrating the cloning of a new effective single R gene against a certain pathotype, the gene is reported to be no longer completely effective due to the appearance of new virulent pathotypes.

The relatives of wheat are a proven source of genetic resistance that can be transferred to commercial cultivars. The globally successful CIMMYT-derived wheats of last century were protected by Sr2 (transferred from Triticum turgidum) and also Sr31 (from Petkus rye). The stem rust resistance locus Sr26 is derived from tall wheat grass (Thinopyrum ponticum (Podp.) Barkworth & D. R. Dewey (Syn. Agropyron elongatum (Host) Beauvoir ssp. ruthenicum Beldie) (2n=10x=70)). Its introgression into wheat as the chromosome wheat-6Ae #1 translocation has been considered as one of the most successful examples of utilization of resistance resources from wheat wild relatives (Knott et al., 1961; Dundas et al., 2015). The Sr26 locus (McIntosh et al., 1995) was transferred to wheat chromosome 6A by Dr Doug Knott of the University of Saskatchewan (Knott et al., 1961) using irradiation techniques, and is a unique resistance that remains effective against all known Pgt pathotypes, including all races from the Ug99 group. Knott et al. (1961) used the wheat-Agropyron (now Thinopyrum) derivative previously developed by L. H. Shebeski and the translocation carrying Sr26 has been released in several Australian wheat cultivars (Park et al., 2009). McIntosh et al. (1995) made special mention of the fact that Sr26, along with Sr2, were two excellent examples of durable stem rust resistance.

It was proposed that this superior durable resistance locus Sr26 was an integrated resistance effect due to a group of R loci in the introgressed Th. ponticum segment, same as the case for Lr13 (Mundt, 2018). Molecular markers have been developed for Sr26, but due to the presence of the Ph1 gene that regulates chromosome pairing and recombination, there is no recombination between wheat and the introduced Th. ponticum chromosome segment. All markers developed so far for Sr26 are potentially physically distant from the gene and lack the specificity to reliably track the Sr26 gene itself. It is particularly difficult to differentiate Sr26 from other genes that are also derived from the Th. ponticum background. Consequently, it has not been possible to determine whether the Sr26 resistance is a single locus or a cluster of resistance loci.

Thus, there is a need to identify the Sr26 gene for use in developing rust resistant plants such as cereal crops.

### SUMMARY OF THE INVENTION

The present inventors have identified a new polypeptide and gene which confer some level of resistance to plants against Puccinia graminis.

Thus, in a first aspect, the present invention provides a plant comprising an exogenous polynucleotide encoding a polypeptide which confers resistance to at least one strain of Puccinia graminis, wherein the polypeptide comprises amino acids having a sequence as provided in SEQ ID NO:1, a biologically active fragment thereof, or an amino acid sequence which is at least 70% identical to SEQ ID NO:1.

In an embodiment, the polynucleotide is operably linked to a promoter capable of directing expression of the polynucleotide in a cell of the plant.

In another aspect, the present invention provides a transgenic plant which has integrated into its genome an exogenous polynucleotide encoding a polypeptide which confers resistance to at least one strain of Puccinia graminis, wherein the polypeptide comprises amino acids having a sequence as provided in SEQ ID NO:1, a biologically active fragment thereof, or an amino acid sequence which is at least 70% identical to SEQ ID NO:1, and wherein the polynucleotide is operably linked to a promoter capable of directing expression of the polynucleotide in a cell of the plant.

In an embodiment, the Puccinia graminis is Puccinia graminis f. sp. tritici.

In an embodiment, the Puccinia graminis f. sp. tritici is a race of Ug99 or DIGALU.

In an embodiment, the strain is one or more or all of race TTRTF, PTKST, TKKTF, TKTTF and PCHSF of Puccinia graminis f. sp. tritici.

In an embodiment, the transgenic plant has enhanced resistance to at least one strain of Puccinia graminis when compared to an isogenic plant lacking the exogenous polynucleotide.

In an embodiment, the polypeptide is an Sr26 polypeptide.

In an embodiment, the polynucleotide comprises nucleotides having a sequence as provided in SEQ ID NO:2, a sequence which is at least 70% identical to SEQ ID NO:2, or a sequence which hybridizes to SEQ ID NO:2. In a further embodiment,

i) the polypeptide comprises amino acids having a sequence which is at least 90% identical to SEQ ID NO:1, and/or

ii) the polynucleotide comprises a sequence which is at least 90% identical to SEQ ID NO:2.

In an embodiment, the polypeptide comprises one or more, preferably all, of a coiled coil (CC) domain, an nucleotide binding (NB) domain and a leucine rich repeat (LRR) domain.

In a further embodiment, the polypeptide comprises one or more, preferably all, of a p-loop motif, a kinase 2 motif and a kinase3a motif in the NB domain.

In an embodiment, the p-loop motif comprises the sequence GxxGxGK(T/S)T (SEQ ID NO:20), more preferably the sequence GSGGMGKTT (SEQ ID NO:21). In an embodiment, the p-loop motif comprises the sequence VSIVGSGGMGKTTL (SEQ ID NO:22).

In an embodiment, the kinase 2 motif comprises the sequence DDxW (SEQ ID NO:23), more preferably the sequence DDIW (SEQ ID NO:24). In an embodiment, the kinase 2 motif comprises the sequence RYFVVLDDIWDVV (SEQ ID NO:25).

In an embodiment, the kinase 3a motif comprises the sequence GxxxxxTxR (SEQ ID NO:26), more preferably the sequence GSIIITTTR (SEQ ID NO:27). In an embodiment, the kinase 3a motif comprises the sequence GSIIITTTRINEV (SEQ ID NO:28).

In a further embodiment, the LRR domain comprises about 5 to about 15 imperfect repeats of the sequence xxLxLxxxx (SEQ ID NO:29).

Preferably, the plant is a cereal plant. Examples of transgenic cereal plants of the invention include, but are not limited to wheat, barley, maize, rice, oats and triticale. In a particularly preferred embodiment, the plant is wheat.

In a further embodiment, the plant comprises one or more further exogenous polynucleotides encoding another plant pathogen resistance polypeptide. Examples of such other plant pathogen resistance polypeptides include, but are not limited to, Lr34, Lr1, Lr3, Lr2a, Lr3ka, Lr11, Lr13, Lr16, Lr17, Lr18, Lr21, LrB, Lr67, Lr46, Sr50, Sr33, Sr13 and Sr35. In an embodiment, the plant further comprises Lr34, Lr67 and Lr46.

Preferably, the plant is homozygous for the exogenous polynucleotide.

In an embodiment, the plant is growing in a field.

Also provided is a population of at least 100 transgenic plants of the invention growing in a field.

In another aspect, the present invention provides a process for identifying a polynucleotide encoding a polypeptide which confers resistance to at least one strain of Puccinia graminis comprising:

i) obtaining a polynucleotide operably linked to a promoter, the polynucleotide encoding a polypeptide comprising amino acids having a sequence as provided in SEQ ID NO:1, a biologically active fragment thereof, or an amino acid sequence which is at least 70% identical to SEQ ID NO:1,

ii) introducing the polynucleotide into a plant,

iii) determining whether the level of resistance to Puccinia graminis is modified relative to an isogenic plant lacking the polynucleotide, and

iv) optionally, selecting a polynucleotide which when expressed confers resistance to Puccinia graminis.

In an embodiment, the polynucleotide comprises nucleotides having a sequence as provided in SEQ ID NO:2, a sequence which is at least 82% identical to SEQ ID NO:2, or a sequence which hybridizes to SEQ ID NO:2.

In another embodiment, the plant is a cereal plant such as a wheat, barley or triticale plant.

In another embodiment, the polypeptide is a plant polypeptide or mutant thereof.

In another embodiment, step ii) further comprises stably integrating the polynucleotide operably linked to a promoter into the genome of the plant.

In an embodiment, the strain is one or more or all of race TTRTF, PTKST, TKKTF, TKTTF and PCHSF of Puccinia graminis f. sp. tritici.

Also provided is a substantially purified and/or recombinant polypeptide which confers resistance to at least one strain of Puccinia graminis, wherein the polypeptide comprises amino acids having a sequence as provided in SEQ ID NO:1, a biologically active fragment thereof, or an amino acid sequence which is at least 70% identical to SEQ ID NO:1

In an embodiment, the polypeptide is an Sr26 polypeptide.

In an embodiment, the polypeptide comprises amino acids having a sequence which is at least 80% identical, at least 90% identical, or at least 95% identical, to SEQ ID NO:1.

In an embodiment, a polypeptide of the invention is a fusion protein further comprising at least one other polypeptide sequence. The at least one other polypeptide may be, for example, a polypeptide that enhances the stability of a polypeptide of the present invention, or a polypeptide that assists in the purification or detection of the fusion protein.

In a further aspect, the present invention provides an isolated and/or exogenous polynucleotide comprising nucleotides having a sequence as provided in SEQ ID NO:2, a sequence which is at least 70% identical to SEQ ID NO:2, a sequence encoding a polypeptide of the invention, or a sequence which hybridizes to SEQ ID NO:2.

In another aspect, the present invention provides a chimeric vector comprising the polynucleotide of the invention. Preferably, the polynucleotide is operably linked to a promoter.

In a further aspect, the present invention provides a recombinant cell comprising an exogenous polynucleotide of the invention and/or a vector of the invention.

The cell can be any cell type such as, but not limited to, a plant cell, a bacterial cell, an animal cell or a yeast cell.

Preferably, the cell is a plant cell. More preferably, the plant cell is a cereal plant cell. Even more preferably, the cereal plant cell is a wheat cell.

In a further aspect, the present invention provides a method of producing the polypeptide of the invention, the method comprising expressing in a cell or cell free expression system the polynucleotide of the invention.

Preferably, the method further comprises isolating the polypeptide.

In yet another aspect, the present invention provides a transgenic non-human organism comprising an exogenous polynucleotide of the invention, a vector of the invention and/or a recombinant cell of the invention.

Preferably, the transgenic non-human organism is a plant. Preferably, the plant is a cereal plant. More preferably, the cereal plant is a wheat plant.

In another aspect, the present invention provides a method of producing the cell of the invention, the method comprising the step of introducing the polynucleotide of the invention, or a vector of the invention, into a cell.

Preferably, the cell is a plant cell.

In a further aspect, the present invention provides a method of producing a transgenic plant of the invention, the method comprising the steps of

i) introducing a polynucleotide of the invention and/or a vector of the invention into a cell of a plant,

ii) regenerating a transgenic plant from the cell, and

iii) optionally harvesting seed from the plant, and/or

iv) optionally producing one or more progeny plants from the transgenic plant, thereby producing the transgenic plant.

In a further aspect, the present invention provides a method of producing a transgenic plant of the invention, the method comprising the steps of

i) crossing two parental plants, wherein at least one plant is a transgenic plant of the invention,

ii) screening one or more progeny plants from the cross for the presence or absence of the polynucleotide, and

iii) selecting a progeny plant which comprise the polynucleotide, thereby producing the plant.

In an embodiment, at least one of the parental plants is a transgenic plant of the invention, and the selected progeny plant comprises an exogenous polynucleotide encoding a polypeptide which confers resistance to at least one strain Puccinia graminis.

In a further embodiment, at least one of the parental plants is a tetraploid or hexaploid wheat plant.

In yet another embodiment, step ii) comprises analysing a sample comprising DNA from the plant for the polynucleotide.

In another embodiment, step iii) comprises

i) selecting progeny plants which are homozygous for the polynucleotide, and/or

ii) analysing the plant or one or more progeny plants thereof for resistance to at least one strain of Puccinia graminis.

In an embodiment, the strain is one or more or all of race TTRTF, PTKST, TKKTF, TKTTF and PCHSF of Puccinia graminis f. sp. tritici.

In an embodiment, the method further comprises

iii) backcrossing the progeny of the cross of step i) with plants of the same genotype as a first parent plant which lacked a polynucleotide encoding a polypeptide which confers resistance to at least one strain of Puccinia graminis for a sufficient number of times to produce a plant with a majority of the genotype of the first parent but comprising the polynucleotide, and

iv) selecting a progeny plant which has resistance to the at least one strain of Puccinia graminis.

In yet another aspect, a method of the invention further comprises the step of analysing the plant for at least one other genetic marker.

Also provided is a plant produced using a method of the invention.

Also provided is the use of the polynucleotide of the invention, or a vector of the invention, to produce a recombinant cell and/or a transgenic plant. In an embodiment, the transgenic plant has enhanced resistance to at least one strain of Puccinia graminis when compared to an isogenic plant lacking the exogenous polynucleotide and/or vector.

In a further aspect, the present invention provides a method for identifying a plant comprising a polynucleotide encoding a polypeptide which confers resistance to at least one strain of Puccinia graminis, the method comprising the steps of

i) obtaining a nucleic acid sample from a plant, and

ii) screening the sample for the presence or absence of the polynucleotide, wherein the polynucleotide encodes a polypeptide of the invention.

In an embodiment, the polynucleotide comprises nucleotides having a sequence as provided in SEQ ID NO:2, a sequence which is at least 70% identical to SEQ ID NO:2, or a sequence which hybridizes to SEQ ID NO:2.

In an embodiment, the method identifies a transgenic plant of the invention.

In another embodiment, the method further comprises producing a plant from a seed before step i).

Also provided is a plant part of the plant of the invention.

In an embodiment, the plant part is a seed that comprises an exogenous polynucleotide which encodes a polypeptide which confers resistance to at least one strain of Puccinia graminis.

In a further aspect, the present invention provides a method of producing a plant part, the method comprising,

a) growing a plant of the invention, and

b) harvesting the plant part.

In another aspect, the present invention provides a method of producing flour, wholemeal, starch or other product obtained from seed, the method comprising;

a) obtaining seed of the invention, and

b) extracting the flour, wholemeal, starch or other product.

In a further aspect, the present invention provides a product produced from a plant of the invention and/or a plant part of the invention.

In an embodiment, the part is a seed.

In an embodiment, the product is a food product or beverage product. Examples include, but are not limited to;

i) the food product being selected from the group consisting of: flour, starch, leavened or unleavened breads, pasta, noodles, animal fodder, animal feed, breakfast cereals, snack foods, cakes, malt, beer, pastries and foods containing flour-based sauces, or

ii) the beverage product being beer or malt.

In an alternative embodiment, the product is a non-food product. Examples include, but are not limited to, films, coatings, adhesives, building materials and packaging materials.

In a further aspect, the present invention provides a method of preparing a food product of the invention, the method comprising mixing seed, or flour, wholemeal or starch from the seed, with another food ingredient.

In another aspect, the present invention provides a method of preparing malt, comprising the step of germinating seed of the invention.

Also provided is the use of a plant of the invention, or part thereof, as animal feed, or to produce feed for animal consumption or food for human consumption.

In a further aspect, the present invention provides a composition comprising one or more of a polypeptide of the invention, a polynucleotide of the invention, a vector of the invention, or a recombinant cell of the invention, and one or more acceptable carriers.

In another aspect, the present invention provides a method of identifying a compound that binds to a polypeptide comprising amino acids having a sequence as provided in SEQ ID NO:1, a biologically active fragment thereof, or an amino acid sequence which is at least 70% identical to SEQ ID NO:1, the method comprising:

i) contacting the polypeptide with a candidate compound, and

ii) determining whether the compound binds the polypeptide.

Any embodiment herein shall be taken to apply mutatis mutandis to any other embodiment unless specifically stated otherwise.

The present invention is not to be limited in scope by the specific embodiments described herein, which are intended for the purpose of exemplification only. Functionally-equivalent products, compositions and methods are clearly within the scope of the invention, as described herein.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

The invention is hereinafter described by way of the following non-limiting Examples and with reference to the accompanying figures.

### BRIEF DESCRIPTION OF THE ACCOMPANYING DRAWINGS

FIG. 1—Phenotypic responses to Puccinia graminis of Sr26 wildtype and mutant 12S line and schematic gene structure of Sr26. a. Wild-type Avocet and Avocet EMS-derived susceptible mutant Sr26 line (12S1 with a S431N mutation inoculated with Pgt isolate PTKST from Ug99 lineage at both seedling and adult plant stages. b. Candidate gene structures (upper), with mutations and their predicted effects on the translated protein identified. Predicted conserved domains for CNL protein are shown corresponding to the gene structure (lower).

FIG. 2—Wildtype and EMS-derived mutants used in the MuRenSeq Pipeline. a. Five mutants and wildtype used for MutRenSeq pipeline to identify Sr26 candidate gene; b. IGV snapshot indicating the SNP changes in each mutant lines employed. The screen capture illustrates the Sr26 locus with four identified susceptible mutants all carrying a mutation in the candidate contig, and one deletion mutant that does not have any reads mapping to the wildtype assembly. The full locus was de novo assembled. From the top to the bottom: Horizontal lines represent the orientation of the identified contig, while read coverage (grey histograms) are indicated on the left, e.g. [0-1651], and the name of line from which the reads are derived on the right. Vertical bars represent the position of the SNPs identified between the reads and reference assembly. Rectangles depict the motifs identified by NLR-Parser (each motif is specific to a conserved NLR domain). Note the orientation of this IGV snapshot view is 3′ to 5′, therefore all the SNPs are actually G to A mutation. Mutant 12S and 70S are likely to be siblings due to possession of identical SNPs.

FIG. 3—The CC (coiled-coil), NB-ARC (Nucleotide binding), and LRR (leucine-rich-repeat) domains are indicated by bars. The conserved motifs (EDVID, Kinase 2, RNBS-B, Kinase 3 (RNBS-C), GLPL, RNBS-D, and MHDV) are indicated by frame and labeled below the sequence. Sequence labeled with stars showing the position of amino acid changes that caused the loss of function mutations. Sr26wtNLR682 and Sr26wtNLR682 are two NLR contigs that have highest similarity with the Sr26 candidate gene from the wildtype de novo assembly. Alignment with other Sr protein sequences Sr13, Sr21, Sr22, Sr33, Sr25, Sr45 and Sr50 is shown.

FIG. 4—Transgenic validation of Sr26. Three constructs at T₀ generation inoculated by Pgt 98-1,2,3,5,6. a. Three constructs used for transformation validation of Sr26 candidate gene. B. Representative phenotypic response to Pgt from T0 plants of each constructs.

FIG. 5—Location of the closest homologs of the Sr26 gene sequence in grass and diploid wheat genomes.

FIG. 6—Phylogenetic analysis of R genes.

FIG. 7—Cell death induction of wheat Sr gene CC domains in planta. (A) Partial alignment of the Sr33, Sr50, Sr26, Sr22, Sr35, Sr45, and Sr46 protein sequences showing the site corresponding to 160 residues of Sr33. (B) Sr331-160, Sr501-163, Sr261-163, Sr221-168, Sr351-161, Sr451-163, and Sr461-171 protein fragments N-terminally fused to YFP were transiently expressed in N. benthamiana. The autoactive Sr50CC-YFP and YFP were used as positive and negative controls, respectively. Cell death was documented 5 days after infiltration. Equivalent results were obtained in three independent experiments. (C) Indicated proteins, transiently expressed in N. benthamiana leaves, were extracted 24 hours after infiltration and analyzed by immunoblotting with anti-GFP antibodies (α-GFP). Ponceau staining of the RubisCO (ribulose-1,5-bisphosphate carboxylase/oxygenase) large subunit shows equal protein loading.

FIG. 8—Sr22 and Sr45 protein fragments without tag were transiently expressed in N. benthamiana. The autoactive Sr50CC-YFP and YFP were used as positive and negative controls, respectively. Cell death was documented 5 days after infiltration. Equivalent results were obtained in three independent experiments.

FIG. 9—Synergistic stem rust resistance observed in wheat seedlings and adult plants inoculated with Pgt pathotype PTKST from Ug99 lineage. Lines labelling order: 1. Kite; 2. Avocet+Lr46; 3. Avocet+Lr34+Lr46+Lr67; 4. Line 37-07 (control); 5. Sr26 mutant 12S; 6. Sr26 mutant 499S. a. Stem rust response observed at 12 dpi at the seedling stage under glasshouse conditions; b. Stem rust response observed at 14 dpi on flag leaves at adult plants under glasshouse conditions; c. First round of stem rust response observed on stems of adult plants under field conditions; d. Second round of stem rust response observed on stems of adult plants under field conditions (21 days after first round); e. Representative colony size differences observed in adult plant flag leaf sheath at 4 dpi under glasshouse conditions; g. Panorama comparison of colony size between Avocet+Lr34+Lr46+Lr67 (No. 3) and Sr26 mutant 12S (No. 5).

FIG. 10—Stem rust responses of flag leaves and stems when inoculated with Pgt pathotype PTKST from Ug99 lineage at the adult plant stage. Lines labelling order: 1. Kite; 2. Avocet+Lr46; 3. Avocet+Lr34+Lr46+Lr67; 4. Line 37-07 (control); 5. Sr26 mutant 12S; 6. Sr26 mutant 499S. a. Stem rust response observed at 20 dpi on flag leaves of adult plants under glasshouse conditions; b. Stem rust response observed at 20 dpi on stems of adults under glasshouse conditions; c. Chitin assay results from the flag leaf sheath at 14 dpi at adult plant stage; d. Average individual colony size measurements from the flag leaf sheath of adult plants at 4 dpi under glasshouse conditions. All results were obtained based on three biological and technical replicates.

### KEY TO THE SEQUENCE LISTING

SEQ ID NO:1—Amino acid sequence of stem rust resistance polypeptide Sr26 polypeptide.

SEQ ID NO:2—Open reading frame encoding Sr26 polypeptide.

SEQ ID NO:3—Amino acid sequence of Sr13 polypeptide (ATE88995.1).

SEQ ID NO:4—Amino acid sequence of Sr21 polypeptide (AVK42833.1).

SEQ ID NO:5—Amino acid sequence of Sr22 polypeptide (CUM44200.1).

SEQ ID NO:6—Amino acid sequence of Sr33 polypeptide (AGQ17386.1).

SEQ ID NO:7—Amino acid sequence of Sr35 polypeptide (AGP75918.1).

SEQ ID NO:8—Amino acid sequence of Sr45 polypeptide (CUM44213.1).

SEQ ID NO:9—Amino acid sequence of Sr50 polypeptide (ALO61074.1).

SEQ ID NO:10—Amino acid sequence of Chinese Spring 6A protein.

SEQ ID NO:11—Amino acid sequence of Chinese Spring 6B protein.

SEQ ID NO:12—Amino acid sequence of Chinese Spring 6C protein.

SEQ ID NO:13—Genomic sequence encoding Sr26 polypeptide.

SEQ ID NO:14—Fragment of Sr33.

SEQ ID NO:15—Fragment of Sr50.

SEQ ID NO:16—Fragment of Sr26.

SEQ ID NO:17—Fragment of Sr22.

SEQ ID NO:18—Fragment of Sr45.

SEQ ID NO:19—Fragment of Sr46.

SEQ ID NO:20—p-loop consensus motif.

SEQ ID NO:21—Sr26 p-loop motif.

SEQ ID NO:22—Sr26 p-loop motif extended.

SEQ ID NO:23—kinase 2 consensus motif.

SEQ ID NO:24—Sr26 kinase 2 motif.

SEQ ID NO:25—Sr26 kinase 2 motif extended.

SEQ ID NO:26—kinase 3a consensus motif.

SEQ ID NO:27—Sr26 kinase 3a motif.

SEQ ID NO:28—Sr26 kinase 3a motif extended.

SEQ ID NO:29—LRR domain repeat consensus sequence.

SEQ ID NO's 30 and 31—Oligonucleotide primers.

### DETAILED DESCRIPTION OF THE INVENTION

### General Techniques and Definitions

Unless specifically defined otherwise, all technical and scientific terms used herein shall be taken to have the same meaning as commonly understood by one of ordinary skill in the art (e.g., in cell culture, molecular genetics, plant molecular biology, protein chemistry, and biochemistry).

Unless otherwise indicated, the recombinant protein, cell culture, and immunological techniques utilized in the present invention are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

The term “and/or”, e.g., “X and/or Y” shall be understood to mean either “X and Y” or “X or Y” and shall be taken to provide explicit support for both meanings or for either meaning.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

### Polypeptides

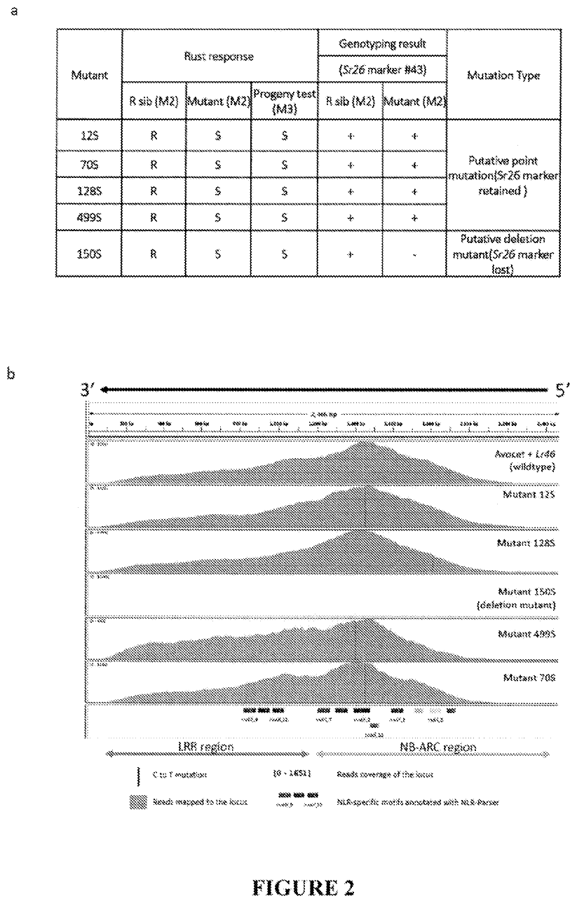

As used herein, the term “Sr26” relates to a protein family which share high primary amino acid sequence identity, for example at least 70%, least 80%, at least 90%, or at least 95% identity with the amino acid sequences provided as SEQ ID NO:1. The present inventors have determined that some variants of the Sr26 protein family, when expressed in a plant, confer upon the plant resistance to at least one strain of Puccinia graminis. An example of such a variant comprises an amino acid sequence provided as SEQ ID NO: 1. Thus, variants which confer resistance are referred to herein as Sr26 (resistant) polypeptides or proteins, whereas those which do not (see as the mutants mentioned in FIG. 2a) are referred to herein as Sr26 (susceptible) polypeptides. In a preferred embodiment, Sr26 (resistant) proteins do not comprise a mutation, such as a threonine, at a position corresponding to amino acid number 311 of SEQ ID NO:1, or a mutation, such as an asparagine, at a position corresponding to amino acid number 431 of SEQ ID NO:1, or a deletion in the RNBS-D motif, such as one or more or all of the amino acids at a position corresponding to amino acid numbers 447 to 468 of SEQ ID NO:1.

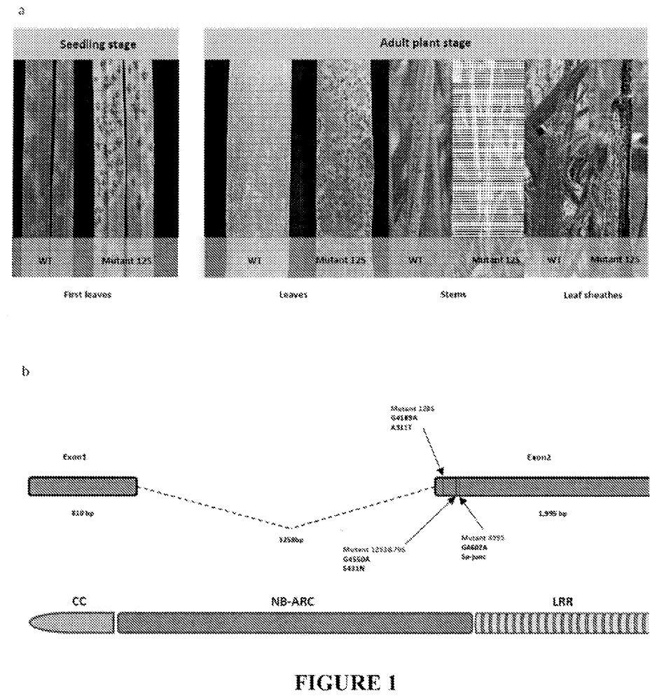

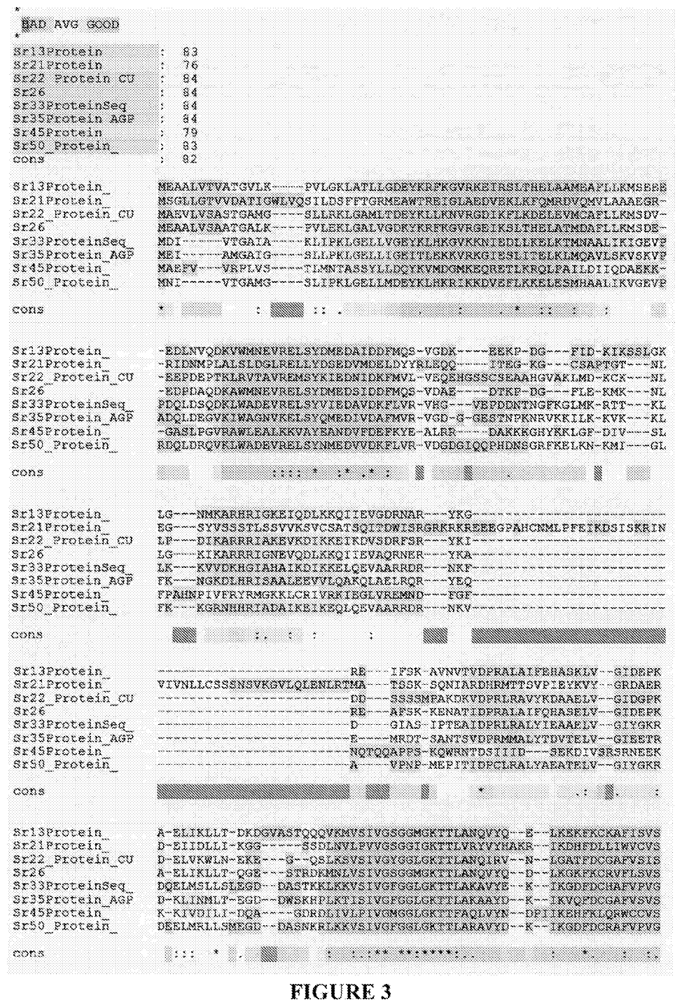

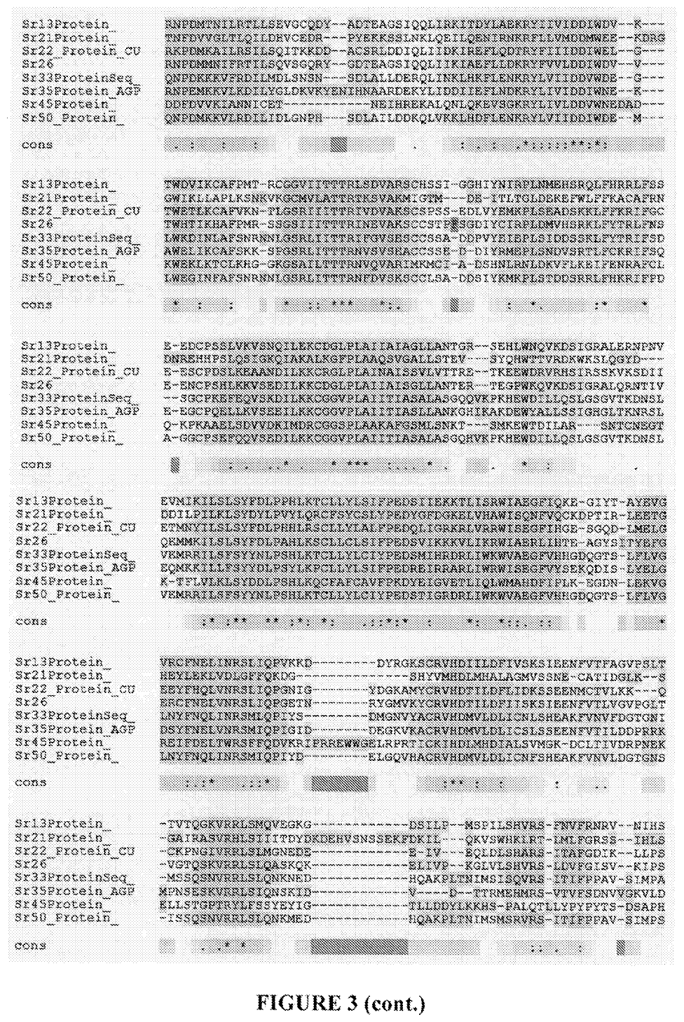

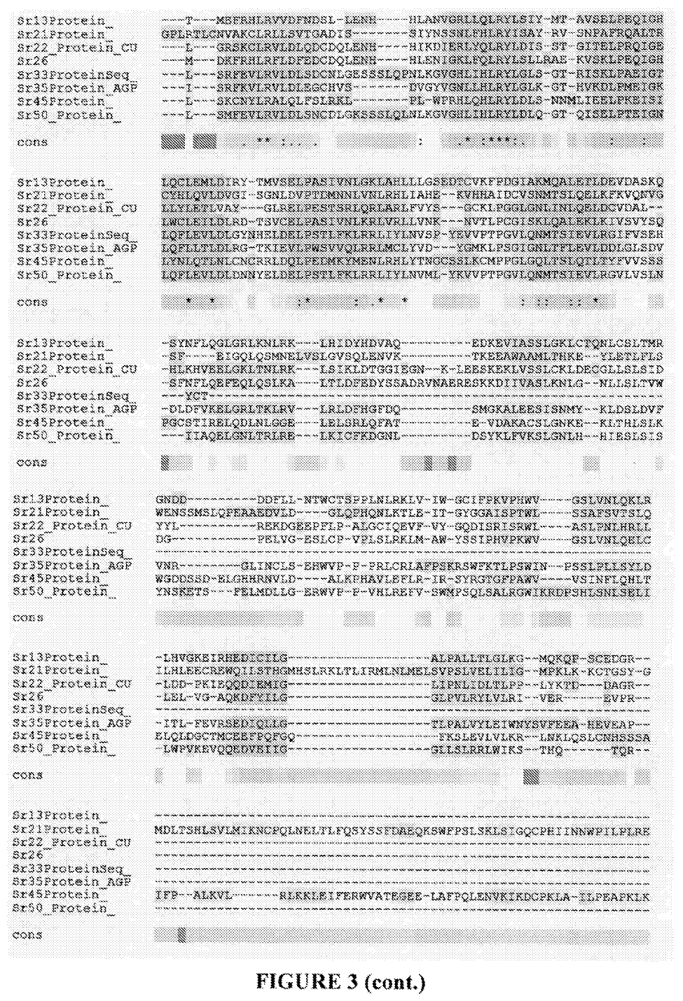

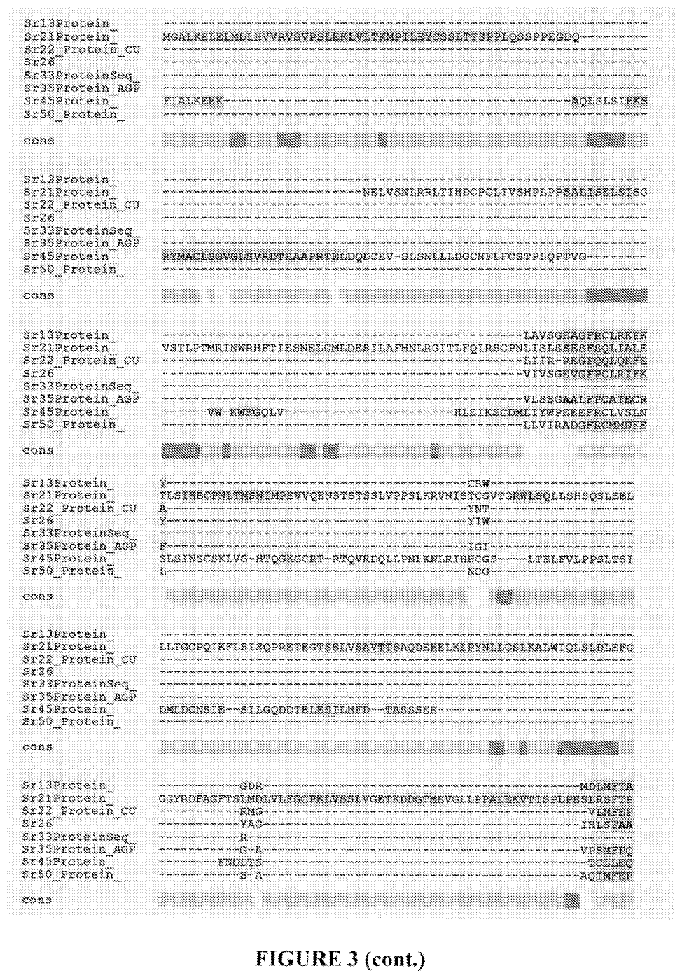

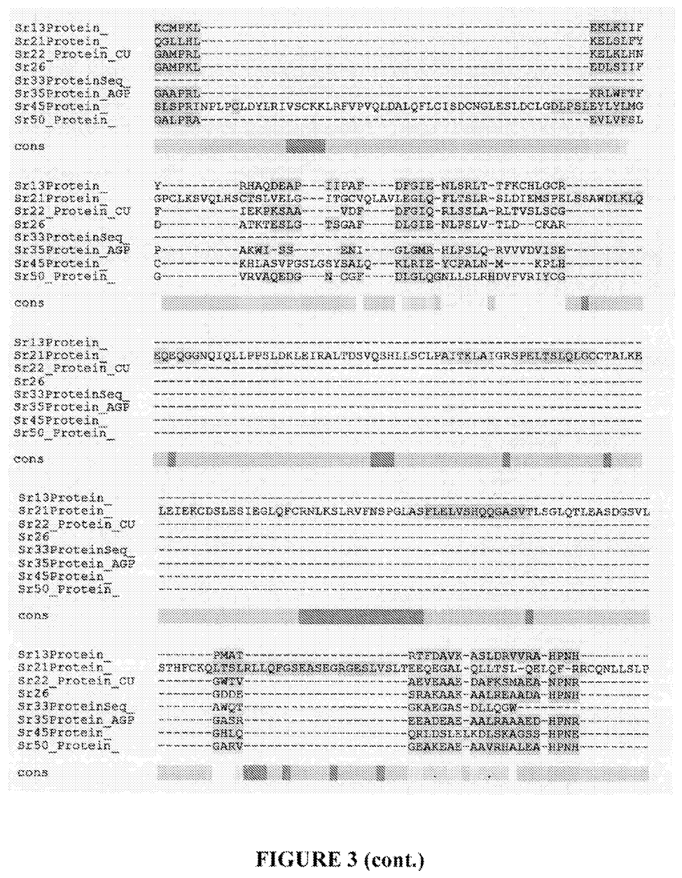

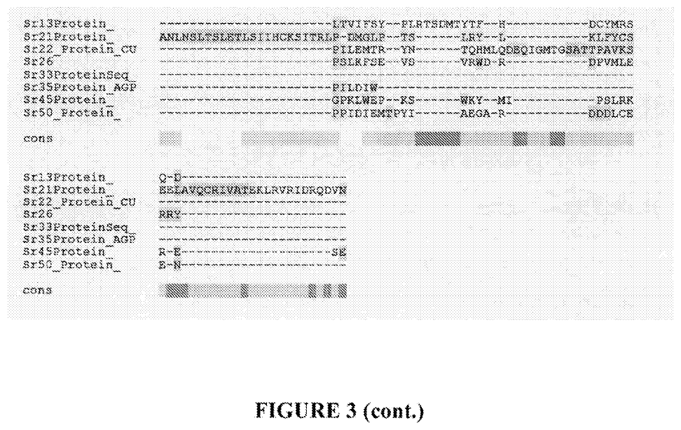

Polypeptides of the invention typically comprise a coiled coil (CC) domain towards the N-terminus, followed by a nucleotide binding (NB) domain and a leucine rich repeat (LRR) domain towards the C-terminus (see FIG. 1b). Each of these three types of domains are common in polypeptides that confer resistance to plant pathogens. In addition, CC-NB-LRR containing polypeptides are a known large class of polypeptides which, as a class, confer resistance across a wide variety of different plant pathogens (see, for example, Bulgarelli et al., 2010; McHale et al., 2006; Takken et al., 2006; Wang et al., 2011; Gennaro et al., 2009; and Dilbirligi et al., 2003), although each CC-NB-LRR polypeptides is specific to a particular species or sub-species of pathogen. Accordingly, by aligning the polypeptides of the invention with other CC-NB-LRR polypeptides, combined with the large number of studies on these types of proteins as well as CC domains, NB domains and LRR domains, the skilled person has a considerable amount of guidance for designing functional variants of the specific polypeptides provided herein (such as provided in FIG. 3).

A coiled-coil domain or motif is a structural motif which is one of the most common tertiary structures of proteins where α-helices are coiled together like the strands of a rope. Computer programs have been devised to detect heptads and resulting in coiled-coil structures (see, for example Delorenzi and Speed, 2002). Coiled coils typically comprise a repeated pattern, hxxhcxc, of hydrophobic (h) and charged (c) amino-acid residues, referred to as a heptad repeats. The positions in the heptad repeat are usually labeled abcdefg, where a and d are the hydrophobic positions, often being occupied by isoleucine, alanine, leucine or valine. Folding a protein with these hepatds into an α-helical secondary structure causes the hydrophobic residues to be presented as a ‘stripe’ that coils gently around the helix in left-handed fashion, forming an amphipathic structure.

The NB domain is present in resistance genes as well as several kinases such as ATP/GTP-binding proteins. This domain typically contains three motifs: kinase-1a (p-loop), a kinase-2, and a putative kinase-3a (Traut 1994; Tameling et al., 2002). The consensus sequence of GxxGxGK(T/S)T (SEQ ID NO:20) (GSGGMGKTT (SEQ ID NO:21) in the polypeptide which confers resistance to Puccinia graminis provided as SEQ ID NO:1), DDxW (SEQ ID NO:23) (DDVW (SEQ ID NO:24) in the polypeptide which confers resistance to Puccinia graminis provided as SEQ ID NO:1) and GxxxxxTxR (SEQ ID NO:26) (GSIIITTTR (SEQ ID NO:27) in the polypeptide which confers resistance to Puccinia graminis provided as SEQ ID NO:1) for the resistance gene motifs p-loop, kinase-2, and the putative kinase-3a, respectively, are different from those present in other NB-encoding proteins. Other motifs present in the NB domain of NB/LRR-type resistance genes are GLPL, RNBS-D and MHD (Meyers et al., 1999). The sequences interspersing these motifs and domains can be very different even among homologues of a resistance gene (Michelmore and Meyers, 1998; Pan et al., 2000).

A leucine-rich domain is a protein structural motif that forms an α/β horseshoe fold (Enkhbayar et al., 2004). The LRR domain contains 9-41 imperfect repeats, each about 25 amino acids long with a consensus amino acid sequence of xxLxLxxxx (SEQ ID NO:29) (Cooley et al., 2000). In an embodiment, a polypeptide of the invention comprises about 5 to about 15, more preferably about 10 to about 14, more preferably about 12 leucine rich repeats. These repeats commonly fold together to form a solenoid protein domain. Typically, each repeat unit has beta strand-turn-alpha helix structure, and the assembled domain, composed of many such repeats, has a horseshoe shape with an interior parallel beta sheet and an exterior array of helices.

As used herein, “resistance” is a relative term in that the presence of a polypeptide of the invention (i) reduces the disease symptoms of a plant comprising the gene (R (resistant) gene) that confers resistance, relative to a plant lacking the R gene, and/or (ii) reduces pathogen reproduction or spread on a plant or within a population of plants comprising the R gene. Resistance as used herein is relative to the “susceptible” response of a plant to the same pathogen. Typically, the presence of the R gene improves at least one production trait of a plant comprising the R gene when infected with the pathogen, such as grain yield, when compared to an isogenic plant infected with the pathogen but lacking the R gene. The isogenic plant may have some level of resistance to the pathogen, or may be classified as susceptible. Thus, the terms “resistance” and “enhanced resistance” are generally used herein interchangeably. Furthermore, a polypeptide of the invention does not necessarily confer complete pathogen resistance, for example when some symptoms still occur or there is some pathogen reproduction on infection but at a reduced amount within a plant or a population of plants. Resistance may occur at only some stages of growth of the plant, for example in adult plants (fully grown in size) and less so, or not at all, in seedlings, or at all stages of plant growth. In an embodiment, resistance occurs at adult and seedling stage. By using a transgenic strategy to express an Sr26 polypeptide in a plant, the plant of the invention can be provided with resistance throughout its growth and development. Enhanced resistance can be determined by a number of methods known in the art such as analysing the plants for the amount of pathogen and/or analysing plant growth or the amount of damage or disease symptoms to a plant in the presence of the pathogen, and comparing one or more of these parameters to an isogenic plant lacking an exogenous gene encoding a polypeptide of the invention.

By “substantially purified polypeptide” or “purified polypeptide” we mean a polypeptide that has generally been separated from the lipids, nucleic acids, other peptides, and other contaminating molecules with which it is associated in its native state. Preferably, the substantially purified polypeptide is at least 90% free from other components with which it is naturally associated. In an embodiment, the polypeptide of the invention has an amino acid sequence which is different to a naturally occurring Sr26 polypeptide i.e. is an amino acid sequence variant.

Transgenic plants and host cells of the invention may comprise an exogenous polynucleotide encoding a polypeptide of the invention. In these instances, the plants and cells produce a recombinant polypeptide. The term “recombinant” in the context of a polypeptide refers to the polypeptide encoded by an exogenous polynucleotide when produced by a cell, which polynucleotide has been introduced into the cell or a progenitor cell by recombinant DNA or RNA techniques such as, for example, transformation. Typically, the cell comprises a non-endogenous gene that causes an altered amount of the polypeptide to be produced. In an embodiment, a “recombinant polypeptide” is a polypeptide made by the expression of an exogenous (recombinant) polynucleotide in a plant cell.

The terms “polypeptide” and “protein” are generally used interchangeably.

The % identity of a polypeptide is determined by GAP (Needleman and Wunsch, 1970) analysis (GCG program) with a gap creation penalty=5, and a gap extension penalty=0.3. The query sequence is at least 500 amino acids in length, and the GAP analysis aligns the two sequences over a region of at least 500 amino acids. More preferably, the query sequence is at least 750 amino acids in length and the GAP analysis aligns the two sequences over a region of at least 750 amino acids. Even more preferably, the query sequence is at least 900 amino acids in length and the GAP analysis aligns the two sequences over a region of at least 900 amino acids. Even more preferably, the GAP analysis aligns two sequences over their entire length, which for an Sr26 polypeptide is about 935 amino acid residues.

As used herein a “biologically active” fragment is a portion of a polypeptide of the invention which maintains a defined activity of the full-length polypeptide such as when expressed in a plant, such as wheat, confers (enhanced) resistance to stem rust caused by at least one strain of Puccinia graminis when compared to an isogenic plant not expressing the polypeptide. Biologically active fragments can be any size as long as they maintain the defined activity but are preferably at least 750 or at least 900 amino acid residues long. Preferably, the biologically active fragment maintains at least 10%, at least 50%, at least 75% or at least 90%, of the activity of the full length protein. In an embodiment, the biologically active fragment comprises functional CC, NB and LRR domains.

With regard to a defined polypeptide, it will be appreciated that % identity figures higher than those provided above will encompass preferred embodiments. Thus, where applicable, in light of the minimum % identity figures, it is preferred that the polypeptide comprises an amino acid sequence which is preferably at least 70%, more preferably at least 75%, more preferably at least 76%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In an embodiment, a polypeptide of the invention is not a naturally occurring polypeptide.

As used herein, the phrase “at a position corresponding to amino acid number” or variations thereof refers to the relative position of the amino acid compared to surrounding amino acids. In this regard, in some embodiments a polypeptide of the invention may have deletional or substitutional mutation which alters the relative positioning of the amino acid when aligned against, for instance, SEQ ID NO:1.

Amino acid sequence mutants of the polypeptides of the present invention can be prepared by introducing appropriate nucleotide changes into a nucleic acid of the present invention, or by in vitro synthesis of the desired polypeptide. Such mutants include, for example, deletions, insertions or substitutions of residues within the amino acid sequence. A combination of deletion, insertion and substitution can be made to arrive at the final construct, provided that the final peptide product possesses the desired characteristics. Preferred amino acid sequence mutants have only one, two, three, four or less than 10 amino acid changes relative to the reference wildtype polypeptide.

Mutant (altered) polypeptides can be prepared using any technique known in the art, for example, using directed evolution or rational design strategies (see below). Products derived from mutated/altered DNA can readily be screened using techniques described herein to determine if, when expressed in a plant, such as wheat, confer (enhanced) resistance to at least one strain of Puccinia graminis. For instance, the method may comprise producing a transgenic plant expressing the mutated/altered DNA and determining the effect of the pathogen on the growth of the plant.

In designing amino acid sequence mutants, the location of the mutation site and the nature of the mutation will depend on characteristic(s) to be modified. The sites for mutation can be modified individually or in series, e.g., by (1) substituting first with conservative amino acid choices and then with more radical selections depending upon the results achieved, (2) deleting the target residue, or (3) inserting other residues adjacent to the located site.

Amino acid sequence deletions generally range from about 1 to 15 residues, more preferably about 1 to 10 residues and typically about 1 to 5 contiguous residues.

Substitution mutants have at least one amino acid residue in the polypeptide molecule removed and a different residue inserted in its place. Where it is desirable to maintain a certain activity it is preferable to make no, or only conservative substitutions, at amino acid positions which are highly conserved in the relevant protein family. Examples of conservative substitutions are shown in Table 1 under the heading of “exemplary substitutions”.

In a preferred embodiment a mutant/variant polypeptide has one or two or three or four conservative amino acid changes when compared to a naturally occurring polypeptide. Details of conservative amino acid changes are provided in Table 1. In a preferred embodiment, the changes are not in one or more of the motifs which are highly conserved between the different polypeptides provided herewith, and/or not in the important motifs of Sr26 polypeptides identified herein. As the skilled person would be aware, such minor changes can reasonably be predicted not to alter the activity of the polypeptide when expressed in a recombinant cell.

The primary amino acid sequence of a polypeptide of the invention can be used to design variants/mutants thereof based on comparisons with closely related polypeptides (for example, as shown in FIG. 3). As the skilled addressee will appreciate, residues highly conserved amongst closely related proteins are less likely to be able to be altered, especially with non-conservative substitutions, and activity maintained than less conserved residues (see above).

TABLE 1
Exemplary substitutions.

| Original | Exemplary |
|---|---|
| Residue | Substitutions |
| Ala (A) | val; leu; ile; gly |
| Arg (R) | lys |
| Asn (N) | gln; his |
| Asp (D) | glu |
| Cys (C) | ser |
| Gln (Q) | asn; his |
| Glu (E) | asp |
| Gly (G) | pro, ala |
| His (H) | asn; gln |
| Ile (I) | leu; val; ala |
| Leu (L) | ile; val; met; ala; phe |
| Lys (K) | arg |
| Met (M) | leu; phe |
| Phe (F) | leu; val; ala |
| Pro (P) | gly |
| Ser (S) | thr |
| Thr (T) | ser |
| Trp (W) | tyr |
| Tyr (Y) | trp; phe |
| Val (V) | ile; leu; met; phe, ala |

Also included within the scope of the invention are polypeptides of the present invention which are differentially modified during or after synthesis, e.g., by biotinylation, benzylation, glycosylation, acetylation, phosphorylation, amidation, derivatization by known protecting/blocking groups, proteolytic cleavage, linkage to an antibody molecule or other cellular ligand, etc. The polypeptides may be post-translationally modified in a cell, for example by phosphorylation, which may modulate its activity. These modifications may serve to increase the stability and/or bioactivity of the polypeptide of the invention.

### Directed Evolution

In directed evolution, random mutagenesis is applied to a protein, and a selection regime is used to pick out variants that have the desired qualities, for example, increased activity. Further rounds of mutation and selection are then applied. A typical directed evolution strategy involves three steps:

1) Diversification: The gene encoding the protein of interest is mutated and/or recombined at random to create a large library of gene variants. Variant gene libraries can be constructed through error prone PCR (see, for example, Leung, 1989; Cadwell and Joyce, 1992), from pools of DNaseI digested fragments prepared from parental templates (Stemmer, 1994a; Stemmer, 1994b; Crameri et al., 1998; Coco et al., 2001) from degenerate oligonucleotides (Ness et al., 2002, Coco, 2002) or from mixtures of both, or even from undigested parental templates (Zhao et al., 1998; Eggert et al., 2005; Jézéquek et al., 2008) and are usually assembled through PCR. Libraries can also be made from parental sequences recombined in vivo or in vitro by either homologous or non-homologous recombination (Ostermeier et al., 1999; Volkov et al., 1999; Sieber et al., 2001). Variant gene libraries can also be constructed by sub-cloning a gene of interest into a suitable vector, transforming the vector into a “mutator” strain such as the E. coli XL-1 red (Stratagene) and propagating the transformed bacteria for a suitable number of generations. Variant gene libraries can also be constructed by subjecting the gene of interest to DNA shuffling (i.e., in vitro homologous recombination of pools of selected mutant genes by random fragmentation and reassembly) as broadly described by Harayama (1998).

2) Selection: The library is tested for the presence of mutants (variants) possessing the desired property using a screen or selection. Screens enable the identification and isolation of high-performing mutants by hand, while selections automatically eliminate all nonfunctional mutants. A screen may involve screening for the presence of known conserved amino acid motifs. Alternatively, or in addition, a screen may involve expressing the mutated polynucleotide in a host organsim or part thereof and assaying the level of activity.

3) Amplification: The variants identified in the selection or screen are replicated many fold, enabling researchers to sequence their DNA in order to understand what mutations have occurred.

Together, these three steps are termed a “round” of directed evolution. Most experiments will entail more than one round. In these experiments, the “winners” of the previous round are diversified in the next round to create a new library. At the end of the experiment, all evolved protein or polynucleotide mutants are characterized using biochemical methods.

### Rational Design

A protein can be designed rationally, on the basis of known information about protein structure and folding. This can be accomplished by design from scratch (de novo design) or by redesign based on native scaffolds (see, for example, Hellinga, 1997; and Lu and Berry, Protein Structure Design and Engineering, Handbook of Proteins 2, 1153-1157 (2007)). Protein design typically involves identifying sequences that fold into a given or target structure and can be accomplished using computer models. Computational protein design algorithms search the sequence-conformation space for sequences that are low in energy when folded to the target structure. Computational protein design algorithms use models of protein energetics to evaluate how mutations would affect a protein's structure and function. These energy functions typically include a combination of molecular mechanics, statistical (i.e. knowledge-based), and other empirical terms. Suitable available software includes IPRO (Interative Protein Redesign and Optimization), EGAD (A Genetic Algorithm for Protein Design), Rosetta Design, Sharpen, and Abalone.

### Polynucleotides and Genes

The present invention refers to various polynucleotides. As used herein, a “polynucleotide” or “nucleic acid” or “nucleic acid molecule” means a polymer of nucleotides, which may be DNA or RNA or a combination thereof, and includes genomic DNA, mRNA, cRNA, and cDNA. Less preferred polynucleotides include tRNA, siRNA, shRNA and hpRNA. It may be DNA or RNA of cellular, genomic or synthetic origin, for example made on an automated synthesizer, and may be combined with carbohydrate, lipids, protein or other materials, labelled with fluorescent or other groups, or attached to a solid support to perform a particular activity defined herein, or comprise one or more modified nucleotides not found in nature, well known to those skilled in the art. The polymer may be single-stranded, essentially double-stranded or partly double-stranded. Basepairing as used herein refers to standard basepairing between nucleotides, including G:U basepairs. “Complementary” means two polynucleotides are capable of basepairing (hybridizing) along part of their lengths, or along the full length of one or both. A “hybridized polynucleotide” means the polynucleotide is actually basepaired to its complement. The term “polynucleotide” is used interchangeably herein with the term “nucleic acid”. Preferred polynucleotides of the invention encode a polypeptide of the invention.

By “isolated polynucleotide” we mean a polynucleotide which has generally been separated from the polynucleotide sequences with which it is associated or linked in its native state, if the polynucleotide is found in nature. Preferably, the isolated polynucleotide is at least 90% free from other components with which it is naturally associated, if it is found in nature. Preferably the polynucleotide is not naturally occurring, for example by covalently joining two shorter polynucleotide sequences in a manner not found in nature (chimeric polynucleotide).

The present invention involves modification of gene activity and the construction and use of chimeric genes. As used herein, the term “gene” includes any deoxyribonucleotide sequence which includes a protein coding region or which is transcribed in a cell but not translated, as well as associated non-coding and regulatory regions. Such associated regions are typically located adjacent to the coding region or the transcribed region on both the 5′ and 3′ ends for a distance of about 2 kb on either side. In this regard, the gene may include control signals such as promoters, enhancers, termination and/or polyadenylation signals that are naturally associated with a given gene, or heterologous control signals in which case the gene is referred to as a “chimeric gene”. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene.

A “Sr26 gene” as used herein refers to a nucleotide sequence which is homologous to an isolated Sr26 cDNA (such as provided in SEQ ID NO:2). As described herein, some alleles and variants of the Sr26 gene family encode a protein that confers resistance to at least one strain of Puccinia graminis. Sr26 genes include the naturally occurring alleles or variants existing in cereals such as wheat, as well as artificially produced variants.

A genomic form or clone of a gene containing the transcribed region may be interrupted with non-coding sequences termed “introns” or “intervening regions” or “intervening sequences”, which may be either homologous or heterologous with respect to the “exons” of the gene. An “intron” as used herein is a segment of a gene which is transcribed as part of a primary RNA transcript but is not present in the mature mRNA molecule. Introns are removed or “spliced out” from the nuclear or primary transcript; introns therefore are absent in the messenger RNA (mRNA). Introns may contain regulatory elements such as enhancers. As described herein, the wheat Sr26 genes (both resistant and susceptible alleles) contain two introns in their protein coding regions. “Exons” as used herein refer to the DNA regions corresponding to the RNA sequences which are present in the mature mRNA or the mature RNA molecule in cases where the RNA molecule is not translated. An mRNA functions during translation to specify the sequence or order of amino acids in a nascent polypeptide. The term “gene” includes a synthetic or fusion molecule encoding all or part of the proteins of the invention described herein and a complementary nucleotide sequence to any one of the above. A gene may be introduced into an appropriate vector for extrachromosomal maintenance in a cell or, preferably, for integration into the host genome.

As used herein, a “chimeric gene” refers to any gene that comprises covalently joined sequences that are not found joined in nature. Typically, a chimeric gene comprises regulatory and transcribed or protein coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. In an embodiment, the protein coding region of an Sr26 gene is operably linked to a promoter or polyadenylation/terminator region which is heterologous to the Sr26 gene, thereby forming a chimeric gene. The term “endogenous” is used herein to refer to a substance that is normally present or produced in an unmodified plant at the same developmental stage as the plant under investigation. An “endogenous gene” refers to a native gene in its natural location in the genome of an organism. As used herein, “recombinant nucleic acid molecule”, “recombinant polynucleotide” or variations thereof refer to a nucleic acid molecule which has been constructed or modified by recombinant DNA/RNA technology. The terms “foreign polynucleotide” or “exogenous polynucleotide” or “heterologous polynucleotide” and the like refer to any nucleic acid which is introduced into the genome of a cell by experimental manipulations.

Foreign or exogenous genes may be genes that are inserted into a non-native organism or cell, native genes introduced into a new location within the native host, or chimeric genes. Alternatively, foreign or exogenous genes may be the result of editing the genome of the organism or cell, or progeny derived therefrom. A “transgene” is a gene that has been introduced into the genome by a transformation procedure. The term “genetically modified” includes introducing genes into cells by transformation or transduction, mutating genes in cells and altering or modulating the regulation of a gene in a cell or organisms to which these acts have been done or their progeny.

Furthermore, the term “exogenous” in the context of a polynucleotide (nucleic acid) refers to the polynucleotide when present in a cell that does not naturally comprise the polynucleotide. The cell may be a cell which comprises a non-endogenous polynucleotide resulting in an altered amount of production of the encoded polypeptide, for example an exogenous polynucleotide which increases the expression of an endogenous polypeptide, or a cell which in its native state does not produce the polypeptide. Increased production of a polypeptide of the invention is also referred to herein as “over-expression”. An exogenous polynucleotide of the invention includes polynucleotides which have not been separated from other components of the transgenic (recombinant) cell, or cell-free expression system, in which it is present, and polynucleotides produced in such cells or cell-free systems which are subsequently purified away from at least some other components. The exogenous polynucleotide (nucleic acid) can be a contiguous stretch of nucleotides existing in nature, or comprise two or more contiguous stretches of nucleotides from different sources (naturally occurring and/or synthetic) joined to form a single polynucleotide. Typically, such chimeric polynucleotides comprise at least an open reading frame encoding a polypeptide of the invention operably linked to a promoter suitable of driving transcription of the open reading frame in a cell of interest.

The % identity of a polynucleotide is determined by GAP (Needleman and Wunsch, 1970) analysis (GCG program) with a gap creation penalty=5, and a gap extension penalty=0.3. The query sequence is at least 450 nucleotides in length, and the GAP analysis aligns the two sequences over a region of at least 450 nucleotides. Preferably, the query sequence is at least 1,500 nucleotides in length, and the GAP analysis aligns the two sequences over a region of at least 1,500 nucleotides. Even more preferably, the query sequence is at least 2,700 nucleotides in length and the GAP analysis aligns the two sequences over a region of at least 2,700 nucleotides. Even more preferably, the GAP analysis aligns two sequences over their entire length.

With regard to the defined polynucleotides, it will be appreciated that % identity figures higher than those provided above will encompass preferred embodiments. Thus, where applicable, in light of the minimum % identity figures, it is preferred that the polynucleotide comprises a polynucleotide sequence which is at least 70%, more preferably at least 75%, more preferably at least 80%, more preferably at least 85%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, more preferably at least 99%, more preferably at least 99.1%, more preferably at least 99.2%, more preferably at least 99.3%, more preferably at least 99.4%, more preferably at least 99.5%, more preferably at least 99.6%, more preferably at least 99.7%, more preferably at least 99.8%, and even more preferably at least 99.9% identical to the relevant nominated SEQ ID NO.

In a further embodiment, the present invention relates to polynucleotides which are substantially identical to those specifically described herein. As used herein, with reference to a polynucleotide the term “substantially identical” means the substitution of one or a few (for example 2, 3, or 4) nucleotides whilst maintaining at least one activity of the native protein encoded by the polynucleotide. In addition, this term includes the addition or deletion of nucleotides which results in the increase or decrease in size of the encoded native protein by one or a few (for example 2, 3, or 4) amino acids whilst maintaining at least one activity of the native protein encoded by the polynucleotide.

The present invention also relates to the use of oligonucleotides, for instance in methods of screening for a polynucleotide of, or encoding a polypeptide of, the invention. As used herein, “oligonucleotides” are polynucleotides up to 50 nucleotides in length. The minimum size of such oligonucleotides is the size required for the formation of a stable hybrid between an oligonucleotide and a complementary sequence on a nucleic acid molecule of the present invention. They can be RNA, DNA, or combinations or derivatives of either. Oligonucleotides are typically relatively short single stranded molecules of 10 to 30 nucleotides, commonly 15-25 nucleotides in length. When used as a guide for genome editing, probe or as a primer in an amplification reaction, the minimum size of such an oligonucleotide is the size required for the formation of a stable hybrid between the oligonucleotide and a complementary sequence on a target nucleic acid molecule. Preferably, the oligonucleotides are at least nucleotides, more preferably at least 18 nucleotides, more preferably at least 19 nucleotides, more preferably at least 20 nucleotides, more preferably at least 22 nucleotides, even more preferably at least 25 nucleotides in length. Oligonucleotides of the present invention used as a probe are typically conjugated with a label such as a radioisotope, an enzyme, biotin, a fluorescent molecule or a chemiluminescent molecule. Examples of oligonucleotides of the invention include those provided in SEQ ID NO's 30 and 31.

The present invention includes oligonucleotides that can be used as, for example, guides for RNA-guided endonucleases, probes to identify nucleic acid molecules, or primers to produce nucleic acid molecules. Probes and/or primers can be used to clone homologues of the polynucleotides of the invention from other species. Furthermore, hybridization techniques known in the art can also be used to screen genomic or cDNA libraries for such homologues.

Polynucleotides and oligonucleotides of the present invention include those which hybridize under stringent conditions to one or more of the sequences provided as SEQ ID NO: 2. As used herein, stringent conditions are those that (1) employ low ionic strength and high temperature for washing, for example, 0.015 M NaCl/0.0015 M sodium citrate/0.1% NaDodSO₄ at 50° C.; (2) employ during hybridisation a denaturing agent such as formamide, for example, 50% (vol/vol) formamide with 0.1% bovine serum albumin, 0.1% Ficoll, 0.1% polyvinylpyrrolidone, 50 mM sodium phosphate buffer at pH 6.5 with 750 mM NaCl, 75 mM sodium citrate at 42° C.; or (3) employ 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 g/ml), 0.1% SDS and 10% dextran sulfate at 42° C. in 0.2×SSC and 0.1% SDS.

Polynucleotides of the present invention may possess, when compared to naturally occurring molecules, one or more mutations which are deletions, insertions, or substitutions of nucleotide residues. Mutants can be either naturally occurring (that is to say, isolated from a natural source) or synthetic (for example, by performing site-directed mutagenesis on the nucleic acid). A variant of a polynucleotide or an oligonucleotide of the invention includes molecules of varying sizes of, and/or are capable of hybridising to, the wheat genome close to that of the reference polynucleotide or oligonucleotide molecules defined herein. For example, variants may comprise additional nucleotides (such as 1, 2, 3, 4, or more), or less nucleotides as long as they still hybridise to the target region. Furthermore, a few nucleotides may be substituted without influencing the ability of the oligonucleotide to hybridise to the target region. In addition, variants may readily be designed which hybridise close to, for example to within 50 nucleotides, the region of the plant genome where the specific oligonucleotides defined herein hybridise. In particular, this includes polynucleotides which encode the same polypeptide or amino acid sequence but which vary in nucleotide sequence by redundancy of the genetic code. The terms “polynucleotide variant” and “variant” also include naturally occurring allelic variants.

### Nucleic Acid Constructs

The present invention includes nucleic acid constructs comprising the polynucleotides of the invention, and vectors and host cells containing these, methods of their production and use, and uses thereof. The present invention refers to elements which are operably connected or linked. “Operably connected” or “operably linked” and the like refer to a linkage of polynucleotide elements in a functional relationship. Typically, operably connected nucleic acid sequences are contiguously linked and, where necessary to join two protein coding regions, contiguous and in reading frame. A coding sequence is “operably connected to” another coding sequence when RNA polymerase will transcribe the two coding sequences into a single RNA, which if translated is then translated into a single polypeptide having amino acids derived from both coding sequences. The coding sequences need not be contiguous to one another so long as the expressed sequences are ultimately processed to produce the desired protein.

As used herein, the term “cis-acting sequence”, “cis-acting element” or “cis-regulatory region” or “regulatory region” or similar term shall be taken to mean any sequence of nucleotides, which when positioned appropriately and connected relative to an expressible genetic sequence, is capable of regulating, at least in part, the expression of the genetic sequence. Those skilled in the art will be aware that a cis-regulatory region may be capable of activating, silencing, enhancing, repressing or otherwise altering the level of expression and/or cell-type-specificity and/or developmental specificity of a gene sequence at the transcriptional or post-transcriptional level. In preferred embodiments of the present invention, the cis-acting sequence is an activator sequence that enhances or stimulates the expression of an expressible genetic sequence.

“Operably connecting” a promoter or enhancer element to a transcribable polynucleotide means placing the transcribable polynucleotide (e.g., protein-encoding polynucleotide or other transcript) under the regulatory control of a promoter, which then controls the transcription of that polynucleotide. In the construction of heterologous promoter/structural gene combinations, it is generally preferred to position a promoter or variant thereof at a distance from the transcription start site of the transcribable polynucleotide which is approximately the same as the distance between that promoter and the protein coding region it controls in its natural setting; i.e., the gene from which the promoter is derived. As is known in the art, some variation in this distance can be accommodated without loss of function. Similarly, the preferred positioning of a regulatory sequence element (e.g., an operator, enhancer etc) with respect to a transcribable polynucleotide to be placed under its control is defined by the positioning of the element in its natural setting; i.e., the genes from which it is derived.

“Promoter” or “promoter sequence” as used herein refers to a region of a gene, generally upstream (5′) of the RNA encoding region, which controls the initiation and level of transcription in the cell of interest. A “promoter” includes the transcriptional regulatory sequences of a classical genomic gene, such as a TATA box and CCAAT box sequences, as well as additional regulatory elements (i.e., upstream activating sequences, enhancers and silencers) that alter gene expression in response to developmental and/or environmental stimuli, or in a tissue-specific or cell-type-specific manner. A promoter is usually, but not necessarily (for example, some PolIII promoters), positioned upstream of a structural gene, the expression of which it regulates. Furthermore, the regulatory elements comprising a promoter are usually positioned within 2 kb of the start site of transcription of the gene. Promoters may contain additional specific regulatory elements, located more distal to the start site to further enhance expression in a cell, and/or to alter the timing or inducibility of expression of a structural gene to which it is operably connected.

“Constitutive promoter” refers to a promoter that directs expression of an operably linked transcribed sequence in many or all tissues of an organism such as a plant. The term constitutive as used herein does not necessarily indicate that a gene is expressed at the same level in all cell types, but that the gene is expressed in a wide range of cell types, although some variation in level is often detectable. “Selective expression” as used herein refers to expression almost exclusively in specific organs of, for example, the plant, such as, for example, endosperm, embryo, leaves, fruit, tubers or root. In a preferred embodiment, a promoter is expressed selectively or preferentially in leaves and/or stems of a plant, preferably a cereal plant. Selective expression may therefore be contrasted with constitutive expression, which refers to expression in many or all tissues of a plant under most or all of the conditions experienced by the plant.

Selective expression may also result in compartmentation of the products of gene expression in specific plant tissues, organs or developmental stages such as adults or seedlings. Compartmentation in specific subcellular locations such as the plastid, cytosol, vacuole, or apoplastic space may be achieved by the inclusion in the structure of the gene product of appropriate signals, eg. a signal peptide, for transport to the required cellular compartment, or in the case of the semi-autonomous organelles (plastids and mitochondria) by integration of the transgene with appropriate regulatory sequences directly into the organelle genome.

A “tissue-specific promoter” or “organ-specific promoter” is a promoter that is preferentially expressed in one tissue or organ relative to many other tissues or organs, preferably most if not all other tissues or organs in, for example, a plant. Typically, the promoter is expressed at a level 10-fold higher in the specific tissue or organ than in other tissues or organs.

In an embodiment, the promoter is a stem-specific promoter, a leaf-specific promoter or a promoter which directs gene expression in an aerial part of the plant (at least stems and leaves) (green tissue specific promoter) such as a ribulose-1,5-bisphosphate carboxylase oxygenase (RUBISCO) promoter.

Examples of stem-specific promoters include, but are not limited to those described in U.S. Pat. No. 5,625,136, and Bam et al. (2008).

The promoters contemplated by the present invention may be native to the host plant to be transformed or may be derived from an alternative source, where the region is functional in the host plant. Other sources include the Agrobacterium T-DNA genes, such as the promoters of genes for the biosynthesis of nopaline, octapine, mannopine, or other opine promoters, tissue specific promoters (see, e.g., U.S. Pat. No. 5,459,252 and WO 91/13992); promoters from viruses (including host specific viruses), or partially or wholly synthetic promoters. Numerous promoters that are functional in mono- and dicotyledonous plants are well known in the art (see, for example, Greve, 1983; Salomon et al., 1984; Garfinkel et al., 1983; Barker et al., 1983); including various promoters isolated from plants and viruses such as the cauliflower mosaic virus promoter (CaMV 35S, 19S). Non-limiting methods for assessing promoter activity are disclosed by Medberry et al. (1992, 1993), Sambrook et al. (1989, supra) and U.S. Pat. No. 5,164,316.

Alternatively, or additionally, the promoter may be an inducible promoter or a developmentally regulated promoter which is capable of driving expression of the introduced polynucleotide at an appropriate developmental stage of the, for example, plant. Other cis-acting sequences which may be employed include transcriptional and/or translational enhancers. Enhancer regions are well known to persons skilled in the art, and can include an ATG translational initiation codon and adjacent sequences. When included, the initiation codon should be in phase with the reading frame of the coding sequence relating to the foreign or exogenous polynucleotide to ensure translation of the entire sequence if it is to be translated. Translational initiation regions may be provided from the source of the transcriptional initiation region, or from a foreign or exogenous polynucleotide. The sequence can also be derived from the source of the promoter selected to drive transcription, and can be specifically modified so as to increase translation of the mRNA.

The nucleic acid construct of the present invention may comprise a 3′ non-translated sequence from about 50 to 1,000 nucleotide base pairs which may include a transcription termination sequence. A 3′ non-translated sequence may contain a transcription termination signal which may or may not include a polyadenylation signal and any other regulatory signals capable of effecting mRNA processing. A polyadenylation signal functions for addition of polyadenylic acid tracts to the 3′ end of a mRNA precursor. Polyadenylation signals are commonly recognized by the presence of homology to the canonical form 5′ AATAAA-3′ although variations are not uncommon. Transcription termination sequences which do not include a polyadenylation signal include terminators for Poll or PolIII RNA polymerase which comprise a run of four or more thymidines. Examples of suitable 3′ non-translated sequences are the 3′ transcribed non-translated regions containing a polyadenylation signal from an octopine synthase (ocs) gene or nopaline synthase (nos) gene of Agrobacterium tumefaciens (Bevan et al., 1983). Suitable 3′ non-translated sequences may also be derived from plant genes such as the ribulose-1,5-bisphosphate carboxylase (ssRUBISCO) gene, although other 3′ elements known to those of skill in the art can also be employed.

As the DNA sequence inserted between the transcription initiation site and the start of the coding sequence, i.e., the untranslated 5′ leader sequence (5′UTR), can influence gene expression if it is translated as well as transcribed, one can also employ a particular leader sequence. Suitable leader sequences include those that comprise sequences selected to direct optimum expression of the foreign or endogenous DNA sequence. For example, such leader sequences include a preferred consensus sequence which can increase or maintain mRNA stability and prevent inappropriate initiation of translation as for example described by Joshi (1987).

### Vectors

The present invention includes use of vectors for manipulation or transfer of genetic constructs. By “chimeric vector” is meant a nucleic acid molecule, preferably a DNA molecule derived, for example, from a plasmid, bacteriophage, or plant virus, into which a nucleic acid sequence may be inserted or cloned. A vector preferably is double-stranded DNA and contains one or more unique restriction sites and may be capable of autonomous replication in a defined host cell including a target cell or tissue or a progenitor cell or tissue thereof, or capable of integration into the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into a cell, is integrated into the genome of the recipient cell and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the cell into which the vector is to be introduced. The vector may also include a selection marker such as an antibiotic resistance gene, a herbicide resistance gene or other gene that can be used for selection of suitable transformants. Examples of such genes are well known to those of skill in the art.

The nucleic acid construct of the invention can be introduced into a vector, such as a plasmid. Plasmid vectors typically include additional nucleic acid sequences that provide for easy selection, amplification, and transformation of the expression cassette in prokaryotic and eukaryotic cells, e.g., pUC-derived vectors, pSK-derived vectors, pGEM-derived vectors, pSP-derived vectors, pBS-derived vectors, or binary vectors containing one or more T-DNA regions. Additional nucleic acid sequences include origins of replication to provide for autonomous replication of the vector, selectable marker genes, preferably encoding antibiotic or herbicide resistance, unique multiple cloning sites providing for multiple sites to insert nucleic acid sequences or genes encoded in the nucleic acid construct, and sequences that enhance transformation of prokaryotic and eukaryotic (especially plant) cells.

By “marker gene” is meant a gene that imparts a distinct phenotype to cells expressing the marker gene and thus allows such transformed cells to be distinguished from cells that do not have the marker. A selectable marker gene confers a trait for which one can “select” based on resistance to a selective agent (e.g., a herbicide, antibiotic, radiation, heat, or other treatment damaging to untransformed cells). A screenable marker gene (or reporter gene) confers a trait that one can identify through observation or testing, i.e., by “screening” (e.g., β-glucuronidase, luciferase, GFP or other enzyme activity not present in untransformed cells). The marker gene and the nucleotide sequence of interest do not have to be linked.

To facilitate identification of transformants, the nucleic acid construct desirably comprises a selectable or screenable marker gene as, or in addition to, the foreign or exogenous polynucleotide. The actual choice of a marker is not crucial as long as it is functional (i.e., selective) in combination with the plant cells of choice. The marker gene and the foreign or exogenous polynucleotide of interest do not have to be linked, since co-transformation of unlinked genes as, for example, described in U.S. Pat. No. 4,399,216 is also an efficient process in plant transformation.

Examples of bacterial selectable markers are markers that confer antibiotic resistance such as ampicillin, erythromycin, chloramphenicol or tetracycline resistance, preferably kanamycin resistance. Exemplary selectable markers for selection of plant transformants include, but are not limited to, a hyg gene which encodes hygromycin B resistance; a neomycin phosphotransferase (nptII) gene conferring resistance to kanamycin, paromomycin, G418; a glutathione-S-transferase gene from rat liver conferring resistance to glutathione derived herbicides as, for example, described in EP 256223; a glutamine synthetase gene conferring, upon overexpression, resistance to glutamine synthetase inhibitors such as phosphinothricin as, for example, described in WO 87/05327, an acetyltransferase gene from Streptomyces viridochromogenes conferring resistance to the selective agent phosphinothricin as, for example, described in EP 275957, a gene encoding a 5-enolshikimate-3-phosphate synthase (EPSPS) conferring tolerance to N-phosphonomethylglycine as, for example, described by Hinchee et al. (1988), a bar gene conferring resistance against bialaphos as, for example, described in WO91/02071; a nitrilase gene such as bxn from Klebsiella ozaenae which confers resistance to bromoxynil (Stalker et al., 1988); a dihydrofolate reductase (DHFR) gene conferring resistance to methotrexate (Thillet et al., 1988); a mutant acetolactate synthase gene (ALS), which confers resistance to imidazolinone, sulfonylurea or other ALS-inhibiting chemicals (EP 154,204); a mutated anthranilate synthase gene that confers resistance to 5-methyl tryptophan; or a dalapon dehalogenase gene that confers resistance to the herbicide.

Preferred screenable markers include, but are not limited to, a uidA gene encoding a β-glucuronidase (GUS) enzyme for which various chromogenic substrates are known, a β-galactosidase gene encoding an enzyme for which chromogenic substrates are known, an aequorin gene (Prasher et al., 1985), which may be employed in calcium-sensitive bioluminescence detection; a green fluorescent protein gene (Niedz et al., 1995) or derivatives thereof; a luciferase (luc) gene (Ow et al., 1986), which allows for bioluminescence detection, and others known in the art. By “reporter molecule” as used in the present specification is meant a molecule that, by its chemical nature, provides an analytically identifiable signal that facilitates determination of promoter activity by reference to protein product.

Preferably, the nucleic acid construct is stably incorporated into the genome of, for example, the plant. Accordingly, the nucleic acid comprises appropriate elements which allow the molecule to be incorporated into the genome, or the construct is placed in an appropriate vector which can be incorporated into a chromosome of a plant cell.

One embodiment of the present invention includes a recombinant vector, which includes at least one polynucleotide molecule of the present invention, inserted into any vector capable of delivering the nucleic acid molecule into a host cell. Such a vector contains heterologous nucleic acid sequences, that is nucleic acid sequences that are not naturally found adjacent to nucleic acid molecules of the present invention and that preferably are derived from a species other than the species from which the nucleic acid molecule(s) are derived. The vector can be either RNA or DNA, either prokaryotic or eukaryotic, and typically is a virus or a plasmid.

A number of vectors suitable for stable transfection of plant cells or for the establishment of transgenic plants have been described in, e.g., Pouwels et al., Cloning Vectors: A Laboratory Manual, 1985, supp. 1987; Weissbach and Weissbach, Methods for Plant Molecular Biology, Academic Press, 1989; and Gelvin et al., Plant Molecular Biology Manual, Kluwer Academic Publishers, 1990. Typically, plant expression vectors include, for example, one or more cloned plant genes under the transcriptional control of 5′ and 3′ regulatory sequences and a dominant selectable marker. Such plant expression vectors also can contain a promoter regulatory region (e.g., a regulatory region controlling inducible or constitutive, environmentally- or developmentally-regulated, or cell- or tissue-specific expression), a transcription initiation start site, a ribosome binding site, an RNA processing signal, a transcription termination site, and/or a polyadenylation signal.

The level of a protein of the invention may be modulated by increasing the level of expression of a nucleotide sequence that codes for the protein in a plant cell, or decreasing the level of expression of a gene encoding the protein in the plant, leading to modified pathogen resistance. The level of expression of a gene may be modulated by altering the copy number per cell, for example by introducing a synthetic genetic construct comprising the coding sequence and a transcriptional control element that is operably connected thereto and that is functional in the cell. A plurality of transformants may be selected and screened for those with a favourable level and/or specificity of transgene expression arising from influences of endogenous sequences in the vicinity of the transgene integration site. A favourable level and pattern of transgene expression is one which results in a substantial modification of pathogen resistance or other phenotype. Alternatively, a population of mutagenized seed or a population of plants from a breeding program may be screened for individual lines with altered pathogen resistance or other phenotype associated with pathogen resistance.

### Recombinant Cells

Another embodiment of the present invention includes a recombinant cell comprising a host cell transformed with one or more recombinant molecules of the present invention, or progeny cells thereof. Transformation of a nucleic acid molecule into a cell can be accomplished by any method by which a nucleic acid molecule can be inserted into the cell. Transformation techniques include, but are not limited to, transfection, particle bombardment/biolistics, electroporation, microinjection, lipofection, adsorption, and protoplast fusion. In an embodiment, gene editing is used to transform the target cell using, for example, targeting nucleases such as TALEN or Cas9-CRISPR.

A recombinant cell may remain unicellular or may grow into a tissue, organ or a multicellular organism. Transformed nucleic acid molecules of the present invention can remain extrachromosomal or can integrate into one or more sites within a chromosome of the transformed (i.e., recombinant) cell in such a manner that their ability to be expressed is retained. Preferred host cells are plant cells, more preferably cells of a cereal plant, more preferably barley or wheat cells, and even more preferably a wheat cell.

### Genome Editing

Endonucleases can be used to generate single strand or double strand breaks in genomic DNA. The genomic DNA breaks in eukaryotic cells are repaired using non-homologous end joining (NHEJ) or homology directed repair (HDR) pathways. NHEJ may result in imperfect repair resulting in unwanted mutations and HDR can enable precise gene insertion by using an exogenous supplied repair DNA template. CRISPR-associated (Cas) proteins have received significant interest although transcription activator-like effector nucleases (TALENs) and zinc-finger nucleases are still useful, the CRISPR-Cas system offers a simpler, versatile and cheaper tool for genome modification (Doudna and Charpentier, 2014).

The CRISPR-Cas systems are classed into three major groups using various nucleases or combinations on nuclease. In class 1 CRISPR-Cas systems (types I, III and IV), the effector module consists of a multi-protein complex whereas class 2 systems (types II, V and VI) use only one effector protein (Makarova et al., 2015). Cas includes a gene that is coupled or close to or localised near the flanking CRISPR loci. Haft et al. (2005) provides a review of the Cas protein family.

The nuclease is guided by the synthetic small guide RNA (sgRNAs or gRNAs) that may or may not include the tracRNA resulting in a simplification of the CRISPR-Cas system to two genes; the endonuclease and the sgRNA (Jinek et al. 2012). The sgRNA is typically under the regulatory control of a U3 or U6 small nuclear RNA promoter. The sgRNA recognises the specific gene and part of the gene for targeting. The protospacer adjacent motif (PAM) is adjacent to the target site constraining the number of potential CRISPR-Cas targets in a genome although the expansion of nucleases also increases the number of PAM's available. There are numerous web tools available for designing gRNAs including CHOPCHOP (http://chopchop.cbu.uib.no), CRISPR design https://omictool s. com/crispr-design-tool, E-CRISP http://www.e-crisp.org/E-CRISP/, Geneious or Benchling https://benchling. com/crispr.

CRISPR-Cas systems are the most frequently adopted in eukaryotic work to date using a Cas9 effector protein typically using the RNA-guided Streptococcus pyogenes Cas9 or an optimised sequence variant in multiple plant species (Luo et al., 2016). Luo et al. (2016) summarises numerous studies where genes have been successfully targeted in various plant species to give rise to indels and loss of function mutant phenotypes in the endogenous gene open reading frame and/or promoter. Due to the cell wall on plant cells the delivery of the CRISPR-Cas machinery into the cell and successful transgenic regenerations have used Agrobacterium tumefaciens infection (Luo et al., 2016) or plasmid DNA particle bombardment or biolistic delivery. Vectors suitable for cereal transformation include pCXUNcas9 (Sun et al, 2016) or pYLCRISPR/Cas9Pubi-H available from Addgene (Ma et al., 2015, accession number KR029109.1).

Alternative CRISPR-Cas systems refer to effector enzymes that contain the nuclease RuvC domain but do not contain the HNH domain including Cas12 enzymes including Cas12a, Cas12b, Cas12f, Cpf1, C2c1, C2c3. Cpf1 creates double-stranded breaks in a staggered manner at the PAM-distal position and being a smaller endonuclease may provide advantages for certain species (Begemann et al., 2017). Other CRISPR-Cas systems include RNA-guided RNAses including Cas13, Cas13a (C2c2), Cas13b, Cas13c.

### Sequence Insertion or Integration

The CRISPR-Cas system can be combined with the provision of a nucleic acid sequence to direct homologous repair for the insertion of a sequence into a genome. Targeted genome integration of plant transgenes enables the sequential addition of transgenes at the same locus. This “cis gene stacking” would greatly simplify subsequent breeding efforts with all transgenes inherited as a single locus. When coupled with CRISPR/Cas9 cleavage of the target site the transgene can be incorporated into this locus by homology-directed repair that is facilitated by flanking sequence homology. This approach can be used to rapidly introduce new alleles without linkage drag or to introduce allelic variants that do not exist naturally.

### Nickases

The CRISPR-Cas II systems use a Cas9 nuclease with two enzymatic cleavage domains a RuvC and HNH domain. Mutations have been shown to alter the double strand cutting to single strand cutting and resulting in a technology variant referred to as a nickase or a nuclease-inactivated Cas9. The RuvC subdomain cleaves the non-complementary DNA strand and the HNH subdomain cleaves that DNA strand complementary to the gRNA. The nickase or nuclease-inactivated Cas9 retains DNA binding ability directed by the gRNA. Mutations in the subdomains are known in the art for example S. pyogenes Cas9 nuclease with a D10A mutation or H840A mutation.

### Genome Base Editing or Modification

Base editors have been created by fusing a deaminase with a Cas9 domain (WO 2018/086623). By fusing the deaminase can take advantage of the sequence targeting directed by the gRNA to make targeted cytidine (C) to uracil (U) conversion by deamination of the cytidine in the DNA. The mismatch repair mechanisms of the cell then replace the U with a T. Suitable cytidine deaminases may include APOBEC1 deaminase, activation-induced cytidine deaminase (AID), APOBEC3G and CDA1. Further, the Cas9-deaminase fusion may be a mutated Cas9 with nickase activity to generate a single strand break. It has been suggested that the nickase protein was potentially more efficient in promoting homology-directed repair (Luo et al., 2016).

### Vector Free Genome Editing or Genome Modification

More recently methods to use vector free approaches using Cas9/sgRNA ribonucleoproteins have been described with successful reduction of off-target events. The method requires in vitro expression of Cas9 ribonucleoproteins (RNPs) which are transformed into the cell or protoplast and does not rely on the Cas9 being integrated into the host genome, thereby reducing the undesirable side cuts that has been linked with the random integration of the Cas9 gene. Only short flanking sequences are required to form a stable Cas9 and sgRNA stable ribonucleoprotein in vitro. Woo et al. (2015) produced pre-assembled Cas9/sgRNA protein/RNA complexes were introduced into protoplasts of Arabidopsis, rice, lettuce and tobacco and targeted mutagenesis frequencies of up to 45% observed in regenerated plants. RNP and in vitro demonstrated in several species including dicot plants (Woo et al., 2015), and monocots maize (Svitashev et al., 2016) and wheat (Liang et al., 2017). Genome editing of plants using CRISPR-Cas 9 in vitro transcripts or ribonucleoproteins are fully described in Liang et al. (2018) and Liang et al. (2019).

### Method for Gene Insertion

Plant embryos may be bombarded with a Cas9 gene and sgRNA gene targeting the site of integration along with the DNA repair template. DNA repair templates are may be synthesised DNA fragment or a 127-mer oligonucleotide, with each encoding the cDNA or the gene of interest. Bombarded cells are grown on tissue culture medium. DNA extracted from callus or TO plants leaf tissue using CTAB DNA extraction method can be analysed by PCR to confirm gene integration. T1 plants selected if per confirms presence of the gene of interest.

The method comprises introducing into a plant cell the DNA sequence of interest referred to as the donor DNA and the endonuclease. The endonuclease generates a break in the target site allowing the first and second regions of homology of the donor DNA to undergo homologous recombination with their corresponding genomic regions of homology. The cut genomic DNA acts as an acceptor of the DNA sequence. The resulting exchange of DNA between the donor and the genome results in the integration of the polynucleotide of interest of the donor DNA into the strand break in the target site in the plant genome, thereby altering the original target site and producing an altered genomic sequence.

The donor DNA may be introduced by any means known in the art. For example, a plant having a target site is provided. The donor DNA may be provided to the plant by known transformation methods including, Agrobacterium-mediated transformation or biolistic particle bombardment. The RNA guided Cas or Cpf1 endonuclease cleaves at the target site, the donor DNA is inserted into the transformed plant's genome.

Although homologous recombination occurs at low frequency in plant somatic cells the process appears to be increased/stimulated by the introduction of doublestrand breaks (DSBs) at selected endonuclease target sites. Ongoing efforts to generate Cas, in particular Cas9, variants or alternatives such as Cpf1 or Cms1 may improve the efficiency.

### Transgenic Plants

The term “plant” as used herein as a noun refers to whole plants and refers to any member of the Kingdom Plantae, but as used as an adjective refers to any substance which is present in, obtained from, derived from, or related to a plant, such as for example, plant organs (e.g. leaves, stems, roots, flowers), single cells (e.g. pollen), seeds, plant cells and the like. Plantlets and germinated seeds from which roots and shoots have emerged are also included within the meaning of “plant”. The term “plant parts” as used herein refers to one or more plant tissues or organs which are obtained from a plant and which comprises genomic DNA of the plant. Plant parts include vegetative structures (for example, leaves, stems), roots, floral organs/structures, seed (including embryo, cotyledons, and seed coat), plant tissue (for example, vascular tissue, ground tissue, and the like), cells and progeny of the same. The term “plant cell” as used herein refers to a cell obtained from a plant or in a plant and includes protoplasts or other cells derived from plants, gamete-producing cells, and cells which regenerate into whole plants. Plant cells may be cells in culture. By “plant tissue” is meant differentiated tissue in a plant or obtained from a plant (“explant”) or undifferentiated tissue derived from immature or mature embryos, seeds, roots, shoots, fruits, tubers, pollen, tumor tissue, such as crown galls, and various forms of aggregations of plant cells in culture, such as calli. Exemplary plant tissues in or from seeds are cotyledon, embryo and embryo axis. The invention accordingly includes plants and plant parts and products comprising these.

As used herein, the term “seed” refers to “mature seed” of a plant, which is either ready for harvesting or has been harvested from the plant, such as is typically harvested commercially in the field, or as “developing seed” which occurs in a plant after fertilisation and prior to seed dormancy being established and before harvest.

A “transgenic plant” as used herein refers to a plant that contains a nucleic acid construct not found in a wild-type plant of the same species, variety or cultivar. That is, transgenic plants (transformed plants) contain genetic material (a transgene) that they did not contain prior to the transformation. The transgene may include genetic sequences obtained from or derived from a plant cell, or another plant cell, or a non-plant source, or a synthetic sequence. Typically, the transgene has been introduced into the plant by human manipulation such as, for example, by transformation but any method can be used as one of skill in the art recognizes. The genetic material is preferably stably integrated into the genome of the plant. The introduced genetic material may comprise sequences that naturally occur in the same species but in a rearranged order or in a different arrangement of elements, for example an antisense sequence. Plants containing such sequences are included herein in “transgenic plants”.

A “non-transgenic plant” is one which has not been genetically modified by the introduction of genetic material by human intervention using, for example, recombinant DNA techniques. In a preferred embodiment, the transgenic plants are homozygous for each and every gene that has been introduced (transgene) so that their progeny do not segregate for the desired phenotype.

As used herein, the term “compared to an isogenic plant”, or similar phrases, refers to a plant which is isogenic relative to the transgenic plant but without the transgene of interest. Preferably, the corresponding non-transgenic plant is of the same cultivar or variety as the progenitor of the transgenic plant of interest, or a sibling plant line which lacks the construct, often termed a “segregant”, or a plant of the same cultivar or variety transformed with an “empty vector” construct, and may be a non-transgenic plant. “Wild type”, as used herein, refers to a cell, tissue or plant that has not been modified according to the invention. Wild-type cells, tissue or plants may be used as controls to compare levels of expression of an exogenous nucleic acid or the extent and nature of trait modification with cells, tissue or plants modified as described herein.

Transgenic plants, as defined in the context of the present invention include progeny of the plants which have been genetically modified using recombinant techniques, wherein the progeny comprise the transgene of interest. Such progeny may be obtained by self-fertilisation of the primary transgenic plant or by crossing such plants with another plant of the same species. This would generally be to modulate the production of at least one protein defined herein in the desired plant or plant organ. Transgenic plant parts include all parts and cells of said plants comprising the transgene such as, for example, cultured tissues, callus and protoplasts.

Plants contemplated for use in the practice of the present invention include both monocotyledons and dicotyledons. Target plants include, but are not limited to, the following: cereals (for example, wheat, barley, rye, oats, rice, maize, sorghum and related crops); grapes; beet (sugar beet and fodder beet); pomes, stone fruit and soft fruit (apples, pears, plums, peaches, almonds, cherries, strawberries, raspberries and black-berries); leguminous plants (beans, lentils, peas, soybeans); oil plants (rape or other Brassicas, mustard, poppy, olives, sunflowers, safflower, flax, coconut, castor oil plants, cocoa beans, groundnuts); cucumber plants (marrows, cucumbers, melons); fibre plants (cotton, flax, hemp, jute); citrus fruit (oranges, lemons, grapefruit, mandarins); vegetables (spinach, lettuce, asparagus, cabbages, carrots, onions, tomatoes, potatoes, paprika); lauraceae (avocados, cinnamon, camphor); or plants such as maize, tobacco, nuts, coffee, sugar cane, tea, vines, hops, turf, bananas and natural rubber plants, as well as ornamentals (flowers, shrubs, broad-leaved trees and evergreens, such as conifers). Preferably, the plant is a cereal plant, more preferably wheat, rice, maize, triticale, oats or barley, even more preferably wheat.

As used herein, the term “wheat” refers to any species of the Genus Triticum, including progenitors thereof, as well as progeny thereof produced by crosses with other species. Wheat includes “hexaploid wheat” which has genome organization of AABBDD, comprised of 42 chromosomes, and “tetraploid wheat” which has genome organization of AABB, comprised of 28 chromosomes. Hexaploid wheat includes T. aestivum, T. spelta, T. macha, T. compactum, T. sphaerococcum, T. vavilovii, and interspecies cross thereof. A preferred species of hexaploid wheat is T. aestivum ssp aestivum (also termed “breadwheat”). Tetraploid wheat includes T. durum (also referred to herein as durum wheat or Triticum turgidum ssp. durum), T. dicoccoides, T. dicoccum, T. polonicum, and interspecies cross thereof. In addition, the term “wheat” includes potential progenitors of hexaploid or tetraploid Triticum sp. such as T. uartu, T. monococcum or T. boeoticum for the A genome, Aegilops speltoides for the B genome, and T. tauschii (also known as Aegilops squarrosa or Aegilops tauschii) for the D genome. Particularly preferred progenitors are those of the A genome, even more preferably the A genome progenitor is T. monococcum. A wheat cultivar for use in the present invention may belong to, but is not limited to, any of the above-listed species. Also encompassed are plants that are produced by conventional techniques using Triticum sp. as a parent in a sexual cross with a non-Triticum species (such as rye [Secale cereale]), including but not limited to Triticale.

As used herein, the term “barley” refers to any species of the Genus Hordeum, including progenitors thereof, as well as progeny thereof produced by crosses with other species. It is preferred that the plant is of a Hordeum species which is commercially cultivated such as, for example, a strain or cultivar or variety of Hordeum vulgare or suitable for commercial production of grain.

Transgenic plants, as defined in the context of the present invention include plants (as well as parts and cells of said plants) and their progeny which have been genetically modified using recombinant techniques to cause production of at least one polypeptide of the present invention in the desired plant or plant organ. Transgenic plants can be produced using techniques known in the art, such as those generally described in A. Slater et al., Plant Biotechnology—The Genetic Manipulation of Plants, Oxford University Press (2003), and P. Christou and H. Klee, Handbook of Plant Biotechnology, John Wiley and Sons (2004).

In a preferred embodiment, the transgenic plants are homozygous for each and every gene that has been introduced (transgene) so that their progeny do not segregate for the desired phenotype. The transgenic plants may also be heterozygous for the introduced transgene(s), such as, for example, in F1 progeny which have been grown from hybrid seed. Such plants may provide advantages such as hybrid vigour, well known in the art.

As used herein, the “other genetic markers” may be any molecules which are linked to a desired trait of a plant. Such markers are well known to those skilled in the art and include molecular markers linked to genes determining traits such disease resistance, yield, plant morphology, grain quality, dormancy traits, grain colour, gibberellic acid content in the seed, plant height, flour colour and the like. Examples of such genes are the stripe rust resistance genes Yr10 or Yr17, the nematode resistance genes such as Cre1 and Cre3, alleles at glutenin loci that determine dough strength such as Ax, Bx, Dx, Ay, By and Dy alleles, the Rht genes that determine a semi-dwarf growth habit and therefore lodging resistance.

Four general methods for direct delivery of a gene into cells have been described: (1) chemical methods (Graham et al., 1973); (2) physical methods such as microinjection (Capecchi, 1980); electroporation (see, for example, WO 87/06614, U.S. Pat. Nos. 5,472,869, 5,384,253, WO 92/09696 and WO 93/21335); and the gene gun (see, for example, U.S. Pat. Nos. 4,945,050 and 5,141,131); (3) viral vectors (Clapp, 1993; Lu et al., 1993; Eglitis et al., 1988); and (4) receptor-mediated mechanisms (Curiel et al., 1992; Wagner et al., 1992).

Acceleration methods that may be used include, for example, microprojectile bombardment and the like. One example of a method for delivering transforming nucleic acid molecules to plant cells is microprojectile bombardment. This method has been reviewed by Yang et al., Particle Bombardment Technology for Gene Transfer, Oxford Press, Oxford, England (1994). Non-biological particles (microprojectiles) that may be coated with nucleic acids and delivered into cells by a propelling force. Exemplary particles include those comprised of tungsten, gold, platinum, and the like. A particular advantage of microprojectile bombardment, in addition to it being an effective means of reproducibly transforming monocots, is that neither the isolation of protoplasts, nor the susceptibility of Agrobacterium infection are required. A particle delivery system suitable for use with the present invention is the helium acceleration PDS-1000/He gun is available from Bio-Rad Laboratories. For the bombardment, immature embryos or derived target cells such as scutella or calli from immature embryos may be arranged on solid culture medium.

In another alternative embodiment, plastids can be stably transformed. Method disclosed for plastid transformation in higher plants include particle gun delivery of DNA containing a selectable marker and targeting of the DNA to the plastid genome through homologous recombination (U.S. Pat. Nos. 5,451,513, 5,545,818, 5,877,402, 5,932,479, and WO 99/05265.

Agrobacterium-mediated transfer is a widely applicable system for introducing genes into plant cells because the DNA can be introduced into whole plant tissues, thereby bypassing the need for regeneration of an intact plant from a protoplast. The use of Agrobacterium-mediated plant integrating vectors to introduce DNA into plant cells is well known in the art (see, for example, U.S. Pat. Nos. 5,177,010, 5,104,310, 5,004,863, 5,159,135). Further, the integration of the T-DNA is a relatively precise process resulting in few rearrangements. The region of DNA to be transferred is defined by the border sequences, and intervening DNA is usually inserted into the plant genome.

Agrobacterium transformation vectors are capable of replication in E. coli as well as Agrobacterium, allowing for convenient manipulations as described (Klee et al., Plant DNA Infectious Agents, Hohn and Schell, (editors), Springer-Verlag, New York, (1985): 179-203). Moreover, technological advances in vectors for Agrobacterium-mediated gene transfer have improved the arrangement of genes and restriction sites in the vectors to facilitate construction of vectors capable of expressing various polypeptide coding genes. The vectors described have convenient multi-linker regions flanked by a promoter and a polyadenylation site for direct expression of inserted polypeptide coding genes and are suitable for present purposes. In addition, Agrobacterium containing both armed and disarmed Ti genes can be used for the transformations. In those plant varieties where Agrobacterium-mediated transformation is efficient, it is the method of choice because of the facile and defined nature of the gene transfer.

A transgenic plant formed using Agrobacterium transformation methods typically contains a single genetic locus on one chromosome. Such transgenic plants can be referred to as being hemizygous for the added gene. More preferred is a transgenic plant that is homozygous for the added structural gene; i.e., a transgenic plant that contains two added genes, one gene at the same locus on each chromosome of a chromosome pair. A homozygous transgenic plant can be obtained by sexually mating (selfing) an independent segregant transgenic plant that contains a single added gene, germinating some of the seed produced and analyzing the resulting plants for the gene of interest.

It is also to be understood that two different transgenic plants can also be mated/crossed to produce offspring that contain two independently segregating exogenous genes. Selfing of appropriate progeny can produce plants that are homozygous for both exogenous genes. Back-crossing to a parental plant and out-crossing with a non-transgenic plant are also contemplated, as is vegetative propagation. Descriptions of other breeding methods that are commonly used for different traits and crops can be found in Fehr, Breeding Methods for Cultivar Development, J. Wilcox (editor) American Society of Agronomy, Madison Wis. (1987).

Transformation of plant protoplasts can be achieved using methods based on calcium phosphate precipitation, polyethylene glycol treatment, electroporation, and combinations of these treatments. Application of these systems to different plant varieties depends upon the ability to regenerate that particular plant strain from protoplasts. Illustrative methods for the regeneration of cereals from protoplasts are described (Fujimura et al., 1985; Toriyama et al., 1986; Abdullah et al., 1986).

Other methods of cell transformation can also be used and include but are not limited to introduction of polynucleotides such as DNA into plants by direct transfer into pollen, by direct injection of polynucleotides such as DNA into reproductive organs of a plant, or by direct injection of polynucleotides such as DNA into the cells of immature embryos followed by the rehydration of desiccated embryos.

The regeneration, development, and cultivation of plants from single plant protoplast transformants or from various transformed explants is well known in the art (Weissbach et al., Methods for Plant Molecular Biology, Academic Press, San Diego, (1988)). This regeneration and growth process typically includes the steps of selection of transformed cells, culturing those individualized cells through the usual stages of embryonic development through the rooted plantlet stage. Transgenic embryos and seeds are similarly regenerated. The resulting transgenic rooted shoots are thereafter planted in an appropriate plant growth medium such as soil.

The development or regeneration of plants containing the foreign, exogenous gene is well known in the art. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired exogenous nucleic acid is cultivated using methods well known to one skilled in the art.

Methods for transforming dicots, primarily by use of Agrobacterium tumefaciens, and obtaining transgenic plants have been published for cotton (U.S. Pat. Nos. 5,004,863, 5,159,135, 5,518,908); soybean (U.S. Pat. Nos. 5,569,834, 5,416,011); Brassica (U.S. Pat. No. 5,463,174); peanut (Cheng et al., 1996); and pea (Grant et al., 1995).

Methods for transformation of cereal plants such as wheat and barley for introducing genetic variation into the plant by introduction of an exogenous nucleic acid and for regeneration of plants from protoplasts or immature plant embryos are well known in the art, see for example, CA 2,092,588, AU 61781/94, AU 667939, U.S. Pat. No. 6,100,447, WO 97/048814, U.S. Pat. Nos. 5,589,617, 6,541,257, and other methods are set out in WO 99/14314. Preferably, transgenic wheat or barley plants are produced by Agrobacterium tumefaciens mediated transformation procedures. Vectors carrying the desired nucleic acid construct may be introduced into regenerable wheat cells of tissue cultured plants or explants, or suitable plant systems such as protoplasts. The regenerable wheat cells are preferably from the scutellum of immature embryos, mature embryos, callus derived from these, or the meristematic tissue.

To confirm the presence of the transgenes in transgenic cells and plants, a polymerase chain reaction (PCR) amplification or Southern blot analysis can be performed using methods known to those skilled in the art. Expression products of the transgenes can be detected in any of a variety of ways, depending upon the nature of the product, and include Western blot and enzyme assay. One particularly useful way to quantitate protein expression and to detect replication in different plant tissues is to use a reporter gene, such as GUS. Once transgenic plants have been obtained, they may be grown to produce plant tissues or parts having the desired phenotype. The plant tissue or plant parts, may be harvested, and/or the seed collected. The seed may serve as a source for growing additional plants with tissues or parts having the desired characteristics.

### Marker Assisted Selection

Marker assisted selection is a well recognised method of selecting for heterozygous plants required when backcrossing with a recurrent parent in a classical breeding program. The population of plants in each backcross generation will be heterozygous for the gene of interest normally present in a 1:1 ratio in a backcross population, and the molecular marker can be used to distinguish the two alleles of the gene. By extracting DNA from, for example, young shoots and testing with a specific marker for the introgressed desirable trait, early selection of plants for further backcrossing is made whilst energy and resources are concentrated on fewer plants. To further speed up the backcrossing program, the embryo from immature seeds (25 days post anthesis) may be excised and grown up on nutrient media under sterile conditions, rather than allowing full seed maturity. This process, termed “embryo rescue”, used in combination with DNA extraction at the three leaf stage and analysis of at least one Sr26 allele or variant that confers upon the plant resistance to at least one strain of Puccinia graminis, allows rapid selection of plants carrying the desired trait, which may be nurtured to maturity in the greenhouse or field for subsequent further backcrossing to the recurrent parent.

Any molecular biological technique known in the art can be used in the methods of the present invention. Such methods include, but are not limited to, the use of nucleic acid amplification, nucleic acid sequencing, nucleic acid hybridization with suitably labelled probes, single-strand conformational analysis (SSCA), denaturing gradient gel electrophoresis (DGGE), heteroduplex analysis (HET), chemical cleavage analysis (CCM), catalytic nucleic acid cleavage or a combination thereof (see, for example, Lemieux, 2000; Langridge et al., 2001). The invention also includes the use of molecular marker techniques to detect polymorphisms linked to alleles of the (for example) Sr26 gene which confers upon the plant resistance to at least one strain of Puccinia graminis. Such methods include the detection or analysis of restriction fragment length polymorphisms (RFLP), RAPD, amplified fragment length polymorphisms (AFLP) and microsatellite (simple sequence repeat, SSR) polymorphisms. The closely linked markers can be obtained readily by methods well known in the art, such as Bulked Segregant Analysis, as reviewed by Langridge et al., (2001).

In an embodiment, a linked loci for marker assisted selection is at least within 1cM, or 0.5cM, or 0.1cM, or 0.01cM from a gene encoding a polypeptide of the invention.

The “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, and typically a thermally-stable polymerase enzyme. Methods for PCR are known in the art, and are taught, for example, in “PCR” (M. J. McPherson and S. G Moller (editors), BIOS Scientific Publishers Ltd, Oxford, (2000)). PCR can be performed on cDNA obtained from reverse transcribing mRNA isolated from plant cells expressing a Sr26 gene or allele which confers upon the plant resistance to at least one strain of Puccinia graminis. However, it will generally be easier if PCR is performed on genomic DNA isolated from a plant.

A primer is an oligonucleotide sequence that is capable of hybridising in a sequence specific fashion to the target sequence and being extended during the PCR. Amplicons or PCR products or PCR fragments or amplification products are extension products that comprise the primer and the newly synthesized copies of the target sequences. Multiplex PCR systems contain multiple sets of primers that result in simultaneous production of more than one amplicon. Primers may be perfectly matched to the target sequence or they may contain internal mismatched bases that can result in the introduction of restriction enzyme or catalytic nucleic acid recognition/cleavage sites in specific target sequences. Primers may also contain additional sequences and/or contain modified or labelled nucleotides to facilitate capture or detection of amplicons. Repeated cycles of heat denaturation of the DNA, annealing of primers to their complementary sequences and extension of the annealed primers with polymerase result in exponential amplification of the target sequence. The terms target or target sequence or template refer to nucleic acid sequences which are amplified.

Methods for direct sequencing of nucleotide sequences are well known to those skilled in the art and can be found for example in Ausubel et al., (supra) and Sambrook et al., (supra). Sequencing can be carried out by any suitable method, for example, dideoxy sequencing, chemical sequencing or variations thereof. Direct sequencing has the advantage of determining variation in any base pair of a particular sequence.

### Tilling

Plants of the invention can be produced using the process known as TILLING (Targeting Induced Local Lesions IN Genomes). In a first step, introduced mutations such as novel single base pair changes are induced in a population of plants by treating seeds (or pollen) with a chemical mutagen, and then advancing plants to a generation where mutations will be stably inherited. DNA is extracted, and seeds are stored from all members of the population to create a resource that can be accessed repeatedly over time.

For a TILLING assay, PCR primers are designed to specifically amplify a single gene target of interest. Specificity is especially important if a target is a member of a gene family or part of a polyploid genome. Next, dye-labeled primers can be used to amplify PCR products from pooled DNA of multiple individuals. These PCR products are denatured and reannealed to allow the formation of mismatched base pairs. Mismatches, or heteroduplexes, represent both naturally occurring single nucleotide polymorphisms (SNPs) (i.e., several plants from the population are likely to carry the same polymorphism) and induced SNPs (i.e., only rare individual plants are likely to display the mutation). After heteroduplex formation, the use of an endonuclease, such as Cel I, that recognizes and cleaves mismatched DNA is the key to discovering novel SNPs within a TILLING population.

Using this approach, many thousands of plants can be screened to identify any individual with a single base change as well as small insertions or deletions (1-30 bp) in any gene or specific region of the genome. Genomic fragments being assayed can range in size anywhere from 0.3 to 1.6 kb. At 8-fold pooling, 1.4 kb fragments (discounting the ends of fragments where SNP detection is problematic due to noise) and 96 lanes per assay, this combination allows up to a million base pairs of genomic DNA to be screened per single assay, making TILLING a high-throughput technique.

TILLING is further described in Slade and Knauf (2005), and Henikoff et al. (2004).

In addition to allowing efficient detection of mutations, high-throughput TILLING technology is ideal for the detection of natural polymorphisms. Therefore, interrogating an unknown homologous DNA by heteroduplexing to a known sequence reveals the number and position of polymorphic sites. Both nucleotide changes and small insertions and deletions are identified, including at least some repeat number polymorphisms. This has been called Ecotilling (Comai et al., 2004).

Each SNP is recorded by its approximate position within a few nucleotides. Thus, each haplotype can be archived based on its mobility. Sequence data can be obtained with a relatively small incremental effort using aliquots of the same amplified DNA that is used for the mismatch-cleavage assay. The left or right sequencing primer for a single reaction is chosen by its proximity to the polymorphism. Sequencher software performs a multiple alignment and discovers the base change, which in each case confirmed the gel band.

Ecotilling can be performed more cheaply than full sequencing, the method currently used for most SNP discovery. Plates containing arrayed ecotypic DNA can be screened rather than pools of DNA from mutagenized plants. Because detection is on gels with nearly base pair resolution and background patterns are uniform across lanes, bands that are of identical size can be matched, thus discovering and genotyping SNPs in a single step. In this way, ultimate sequencing of the SNP is simple and efficient, made more so by the fact that the aliquots of the same PCR products used for screening can be subjected to DNA sequencing.

### Plant/Grain Processing

Grain/seed of the invention, preferably cereal grain and more preferably wheat grain, or other plant parts of the invention, can be processed to produce a food ingredient, food or non-food product using any technique known in the art.

In one embodiment, the product is whole grain flour such as, for example, an ultrafine-milled whole grain flour, or a flour made from about 100% of the grain. The whole grain flour includes a refined flour constituent (refined flour or refined flour) and a coarse fraction (an ultrafine-milled coarse fraction).

Refined flour may be flour which is prepared, for example, by grinding and bolting cleaned grain such as wheat or barley grain. The particle size of refined flour is described as flour in which not less than 98% passes through a cloth having openings not larger than those of woven wire cloth designated “212 micrometers (U.S. Wire 70)”. The coarse fraction includes at least one of: bran and germ. For instance, the germ is an embryonic plant found within the grain kernel. The germ includes lipids, fiber, vitamins, protein, minerals and phytonutrients, such as flavonoids. The bran includes several cell layers and has a significant amount of lipids, fiber, vitamins, protein, minerals and phytonutrients, such as flavonoids. Further, the coarse fraction may include an aleurone layer which also includes lipids, fiber, vitamins, protein, minerals and phytonutrients, such as flavonoids. The aleurone layer, while technically considered part of the endosperm, exhibits many of the same characteristics as the bran and therefore is typically removed with the bran and germ during the milling process. The aleurone layer contains proteins, vitamins and phytonutrients, such as ferulic acid.

Further, the coarse fraction may be blended with the refined flour constituent. The coarse fraction may be mixed with the refined flour constituent to form the whole grain flour, thus providing a whole grain flour with increased nutritional value, fiber content, and antioxidant capacity as compared to refined flour. For example, the coarse fraction or whole grain flour may be used in various amounts to replace refined or whole grain flour in baked goods, snack products, and food products. The whole grain flour of the present invention (i.e. —ultrafine-milled whole grain flour) may also be marketed directly to consumers for use in their homemade baked products. In an exemplary embodiment, a granulation profile of the whole grain flour is such that 98% of particles by weight of the whole grain flour are less than 212 micrometers.

In further embodiments, enzymes found within the bran and germ of the whole grain flour and/or coarse fraction are inactivated in order to stabilize the whole grain flour and/or coarse fraction. Stabilization is a process that uses steam, heat, radiation, or other treatments to inactivate the enzymes found in the bran and germ layer. Flour that has been stabilized retains its cooking characteristics and has a longer shelf life.

In additional embodiments, the whole grain flour, the coarse fraction, or the refined flour may be a component (ingredient) of a food product and may be used to product a food product. For example, the food product may be a bagel, a biscuit, a bread, a bun, a croissant, a dumpling, an English muffin, a muffin, a pita bread, a quickbread, a refrigerated/frozen dough product, dough, baked beans, a burrito, chili, a taco, a tamale, a tortilla, a pot pie, a ready to eat cereal, a ready to eat meal, stuffing, a microwaveable meal, a brownie, a cake, a cheesecake, a coffee cake, a cookie, a dessert, a pastry, a sweet roll, a candy bar, a pie crust, pie filling, baby food, a baking mix, a batter, a breading, a gravy mix, a meat extender, a meat substitute, a seasoning mix, a soup mix, a gravy, a roux, a salad dressing, a soup, sour cream, a noodle, a pasta, ramen noodles, chow mein noodles, lo mein noodles, an ice cream inclusion, an ice cream bar, an ice cream cone, an ice cream sandwich, a cracker, a crouton, a doughnut, an egg roll, an extruded snack, a fruit and grain bar, a microwaveable snack product, a nutritional bar, a pancake, a par-baked bakery product, a pretzel, a pudding, a granola-based product, a snack chip, a snack food, a snack mix, a waffle, a pizza crust, animal food or pet food.

In alternative embodiments, the whole grain flour, refined flour, or coarse fraction may be a component of a nutritional supplement. For instance, the nutritional supplement may be a product that is added to the diet containing one or more additional ingredients, typically including: vitamins, minerals, herbs, amino acids, enzymes, antioxidants, herbs, spices, probiotics, extracts, prebiotics and fiber. The whole grain flour, refined flour or coarse fraction of the present invention includes vitamins, minerals, amino acids, enzymes, and fiber. For instance, the coarse fraction contains a concentrated amount of dietary fiber as well as other essential nutrients, such as B-vitamins, selenium, chromium, manganese, magnesium, and antioxidants, which are essential for a healthy diet. For example 22 grams of the coarse fraction of the present invention delivers 33% of an individual's daily recommend consumption of fiber. The nutritional supplement may include any known nutritional ingredients that will aid in the overall health of an individual, examples include but are not limited to vitamins, minerals, other fiber components, fatty acids, antioxidants, amino acids, peptides, proteins, lutein, ribose, omega-3 fatty acids, and/or other nutritional ingredients. The supplement may be delivered in, but is not limited to the following forms: instant beverage mixes, ready-to-drink beverages, nutritional bars, wafers, cookies, crackers, gel shots, capsules, chews, chewable tablets, and pills. One embodiment delivers the fiber supplement in the form of a flavored shake or malt type beverage, this embodiment may be particularly attractive as a fiber supplement for children.

In an additional embodiment, a milling process may be used to make a multi-grain flour or a multi-grain coarse fraction. For example, bran and germ from one type of grain may be ground and blended with ground endosperm or whole grain cereal flour of another type of cereal. Alternatively, bran and germ of one type of grain may be ground and blended with ground endosperm or whole grain flour of another type of grain. It is contemplated that the present invention encompasses mixing any combination of one or more of bran, germ, endosperm, and whole grain flour of one or more grains. This multi-grain approach may be used to make custom flour and capitalize on the qualities and nutritional contents of multiple types of cereal grains to make one flour.

It is contemplated that the whole grain flour, coarse fraction and/or grain products of the present invention may be produced by any milling process known in the art. An exemplary embodiment involves grinding grain in a single stream without separating endosperm, bran, and germ of the grain into separate streams. Clean and tempered grain is conveyed to a first passage grinder, such as a hammermill, roller mill, pin mill, impact mill, disc mill, air attrition mill, gap mill, or the like. After grinding, the grain is discharged and conveyed to a sifter. Further, it is contemplated that the whole grain flour, coarse fraction and/or grain products of the present invention may be modified or enhanced by way of numerous other processes such as: fermentation, instantizing, extrusion, encapsulation, toasting, roasting, or the like.

### Malting

A malt-based beverage provided by the present invention involves alcohol beverages (including distilled beverages) and non-alcohol beverages that are produced by using malt as a part or whole of their starting material. Examples include beer, happoshu (low-malt beer beverage), whisky, low-alcohol malt-based beverages (e.g., malt-based beverages containing less than 1% of alcohols), and non-alcohol beverages.

Malting is a process of controlled steeping and germination followed by drying of the grain such as barley and wheat grain. This sequence of events is important for the synthesis of numerous enzymes that cause grain modification, a process that principally depolymerizes the dead endosperm cell walls and mobilizes the grain nutrients. In the subsequent drying process, flavour and colour are produced due to chemical browning reactions. Although the primary use of malt is for beverage production, it can also be utilized in other industrial processes, for example as an enzyme source in the baking industry, or as a flavouring and colouring agent in the food industry, for example as malt or as a malt flour, or indirectly as a malt syrup, etc.

In one embodiment, the present invention relates to methods of producing a malt composition. The method preferably comprises the steps of:

(i) providing grain, such as barley or wheat grain, of the invention,

(ii) steeping said grain,

(iii) germinating the steeped grains under predetermined conditions and

(iv) drying said germinated grains.

For example, the malt may be produced by any of the methods described in Hoseney (Principles of Cereal Science and Technology, Second Edition, 1994: American Association of Cereal Chemists, St. Paul, Minn.). However, any other suitable method for producing malt may also be used with the present invention, such as methods for production of speciality malts, including, but limited to, methods of roasting the malt.

Malt is mainly used for brewing beer, but also for the production of distilled spirits. Brewing comprises wort production, main and secondary fermentations and post-treatment. First the malt is milled, stirred into water and heated. During this “mashing”, the enzymes activated in the malting degrade the starch of the kernel into fermentable sugars. The produced wort is clarified, yeast is added, the mixture is fermented and a post-treatment is performed.

### EXAMPLES

### Example 1—Material and Methods

### MutRenSeq Pipeline

### Plant Materials and Mutant DNA Preparation

Seeds of line Avocet+Lr46 (Avocet carries Sr26) were treated with ethyl methanesulfonate (EMS) following the protocol described by Mago et al. (2005). A kill-curve on 20-grains was initially produced with different concentrations, 0.2, 0.4, 0.6, 0.7 and 1.0% (v/v) to identify the dosage required to achieve 50% mortality. M2 families obtained as a single spike progeny from each M1 plant were tested for stem rust response. Individual plants from segregating progenies were grown and progeny tested. Homozygous susceptible mutant and resistant sib pairs were recovered from these progenies.

Genomic DNA was isolated from non-diseased leaves of selected seedlings following the protocol described by Yu et al. (2017). The quality and quantity of DNA were checked with a NanoDrop spectrophotometer (Thermo Scientific) first and then on a 0.8% agarose gel.

### Resistance Gene Enrichment and Sequencing (RenSeq)

The Target enrichment of NLRs was performed by Arbor Biosciences (Ann Arbor, USA) following the MYbaits protocol using an improved version of the previously published Triticeae bait library available at github.com/steuernb/MutantHunter. Library construction was done by following the TruSeq RNA protocol v2. All enriched libraries were sequenced on a HiSeq 2500 (Illumina) using 250 bp paired end reads and SBS chemistry.

### MutantHunter

To identify Sr26 contig from mutants, the inventors followed the MutantHunter pipeline with all default parameters Steuernage et al. (2016), except in the use of CLC Genomics Workbench (V9) for reads QC, trimming, de novo assembly of Avocet wild-type and mapping all the reads against de novo wild-type assembly. Mutants M1 and M5 were likely to be siblings because they shared the same mutated SNP.

### Gene Full-Length Obtaining, Candidate Contig Confirmation, and Gene Structure Confirmation

Total RNA was extracted using the PureLink™ RNA Mini Kit (Invitrogen) as per manufacture instructions. cDNA synthesis were performed using the method described by manufacture (Clontech). The full length of gene was amplified by the 5′ and 3′ RACE (rapid amplification of cDNA ends) kit (Clontech). The 5′ and 3′ untranslated region (UTRs) were obtained by genomic walking kit (Clontech). All mutants used in the RenSeq pipeline were re-confirmed by Sanger sequencing, and each unique SNP from the four mutants led to an amino acid substitution or a splice junction. Predicted exon-intron structures were confirmed by full cDNA amplification and RNA-seq data.

### Transgenic Validation

The Sr26 gene was introduced into wheat cultivar Fielder through binary vector pVecBARII using the Agrobacterium-transformation protocol described by Ishida et al. (2015) and phosphinothricin as a selective agent. TO shoots were transplanted from petridish into a growth cabinet set with day and night temperature of 23° C., 16 hours light and 8 hours dark. Plants were inoculated with Pgt races at 7-10 days after transplanting and scored at 10-15 days as described by McIntosh (1995).

### Phenotyping Under Glasshouse and Field Conditions

Phenotyping of the stem rust responses of seedlings and adult plants in either the glasshouse or field was carried out according to the methods described by Bender et al. (2016) and Pretorius et al. (2015).

### Chitin Assay and Histological Assessment

The chitin assay was carried out according to the protocol described by Ayliffe (2013). Results were based on three biological and technical replicates. For histological assessment, average individual colony size measurements were carried out according to the protocol described by Ayliffe (2013), except that plant tissue was not weighed during sampling. After adding KOH, tubes containing plant tissue were kept at 60° C. overnight before washing 3 times with 50 Mm Tris (pH 7.0). Three to 6 ml of 50 Mm Tris was added to samples after washing. The 1 mg/ml solution of wheat germ agglutinin (WGA) FITC probe (Sigma Aldrich) dissolved in water was then added at a concentration of 7 ul/ml and allowed to stain for 1.5 h.

The measurement of individual colonies of each sample was done with WU epifluorescence cube (450-480 nm excitation filter and 515 nm barrier filter) on an Olympus AX70 microscope (Tokyo, Japan). The length and width of fluorescing colonies were measured to obtain an approximation of colony size (μm2). Microscopic images were captured using a CC12 digital camera and AnalySIS LS Research version 2.2 software (Olympus Soft Imaging System, Japan). The mean size of 15-20 infection sites per sample, each replicated in three independent treatments, was calculated.

### Construction of Phylogenetic Tree

R gene protein sequences found in the NCBI database were aligned using T-coffee and the phylogenetic tree generated using Mega7.

### CC Domain Prediction and CC Conserved Domain Alignment

The coiled-coil domains were determined using the COILS prediction program (Lupas et al., 1991) (https://embnet.vital-it.ch/software/COILS_form.html). The Expresso from T-Coffee program (http://tcoffee.crg.cat/apps/tcoffee/do:expresso) was used for protein sequence alignment.

### Plant Growth Conditions and Transient Expression Analyses

N. benthamiana plants were grown in a growth chamber at 23° C. with a 16-h light period. For transient expression analyses in N. benthamiana, pBIN19-derived vector constructs were transformed into Agrobacterium tumefaciens strain GV3101_pMP90, and the pAM-PAT vector constructs were transformed into GV3103. Bacterial strains were grown in Luria-Bertani liquid medium containing 50 mg/mL rifampicin, 15 mg/mL gentamycin, and 25 mg/mL kanamycin (and 25 mg/mL of carbenicillin for pAM-PAT vectors) at 28° C. for 24 h. Bacteria were harvested by centrifugation, resuspended in infiltration medium [10 mM IVIES (pH 5.6), 10 mM MgCl2, and 150 μM acetosyringone] to an OD600 ranging from 0.5 to 1, and incubated for 2 h at room temperature before leaf infiltration. For each independent infiltration experiment, each construct was infiltrated on three leaves from three or four individual plants. The infiltrated plants were incubated in growth chambers under controlled conditions for all following assays. For documentation of cell death, leaves were photographed 2-5 d after infiltration.

### Construct Generation, Protein Exaction and Immunoblot

CC domains from Sr polypeptides were aligned to the first 160 amino acids of the Sr33 polypeptide sequence. The selected CC domain was fused with its native stop codon at the C-terminus by PCR and cloned into pDonor vector and later transferred into destination vectors pB1N19 with N-terminal YFP fusions by Gateway cloning (Supplier Invitrogen®). Sequences were checked after each transformation. Protein extraction from N. benthamiana leaves was performed as described (Cesari et al., 2013). For immunoblotting analysis, proteins were separated by SDS/PAGE and transferred to a nitrocellulose membrane (Pall). Membranes were blocked in 5% skimmed milk and probed with anti-HA (Roche anti-HA 12CA5 or Roche anti-HA-HRP 3F10), anti-GFP (Roche). Labelling was detected using the SuperSignal West Femto chemiluminescence kit (Pierce). Membranes were stained with Ponceau S to confirm equal loading.

### Gene-Specific Marker

A panel of wheat genetic stocks that were postulated to possess Sr26 were used for validating the gene-specific primers. A primer set that was designed flanking the junction of intron I and exon II, with an amplicon size of 1,580 bp, confirmed to be highly specific for the target gene (Sr26GSPF; 5′-GGAATACTCGAATACCAGGCCAT-3′ (SEQ ID NO:30); Sr26GSPR; 5′-TTGCCACTGTGAACATGTTTATAGAT-3′ (SEQ ID NO:31)).

### Example 2—Cloning Sr26

The inventors identified susceptible ethyl methanesulfonate-derived (EMS) mutants from the Avocet+Lr46 background. Five independent mutants (four with putative point mutations and one with a putative deletion) together with wild-type Avocet+Lr46 were used in a RenSeq pipeline (FIG. 1a and FIG. 2a).

A single contig of 2,470 bp using MutantHunter (Steuernage et al., 2016) was identified (FIG. 2b). The entire full sequence of Sr26 is 6,066 bp consisting of two exons and an intron of 3,258 bp. The encoded 935 amino acid protein contains a coiled-coil (CC) domain at the N-terminus, followed by the NB-ARC domain and then the LRR motifs at the C-terminus (FIG. 1b).

All seven cloned wheat stem rust race-specific R protein sequences Sr13, Sr21, Sr22, Sr33, Sr35, Sr45 and Sr50 were aligned with Sr26 and its homologs in chromosome 6A, 6B, and 6D from CSrefv1.0 by Expresso using structural information. The CC, NB-ARC, LRR domains and conserved motifs were all aligned as indicated in (FIG. 3). All amino acid changes caused by the EMS mutation of Sr26 were located in the conserved motifs of the NB-ARC domain. Mutant 128S1 has an Alanine to Threonine change within the RNBS-C motif, whereas Mutant 70S1 (and mutant 12S1) has a Serine to Asparagine change in RNBS-D motif. Mutant 499S1 has an alternative splicing form that caused a 22 amino acid deletion in motif RNBS-D (FIG. 3).

### Example 3—Transgenic Validation of Sr26

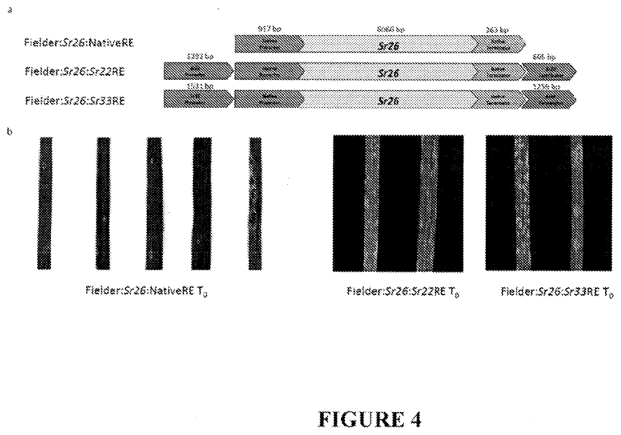

A complementary transgenic experiment was performed to clarify whether the Sr26 candidate gene was responsible for resistance in wheat. Due to the initially obtained 5′ and 3′ UTRs being less than 1 kb (917 bp and 263 bp respectively), there was a potential risk of insufficient regulatory elements that may affect the appropriate gene expression. To ensure expression of the candidate gene, three constructs were used to produce transgenic wheat (FIG. 4a). One construct was assembled with the obtained native 5′ and 3′ UTRs and designated as Fielder:Sr26:NativeRE (Regulatory Elements). The other two constructs, designated as Fielder:Sr26:Sr22RE and Fielder:Sr26:Sr33RE, were fused with the obtained native 5′ and 3′ UTRs together with either the gene regulatory elements from Sr22 or Sr33. Twenty one, 22 and 14 independent primary transgenic lines carrying the Fielder:Sr26:NativeRE, Fielder: Sr26: Sr22RE and Fielder:Sr26:Sr33RE, respectively.

All independent primary transgenic TO plants from Fielder:Sr26:NativeRE, Fielder:Sr26:Sr22RE, and Fielder:Sr26:Sr33RE showed resistance to stem rust pathotype 98-1,2,3,5,6 while all the empty vector transformed Fielder controls were susceptible (FIG. 4b, Table 2).

To test rust responses of Sr26 against the newly emerged Pgt pathotypes, Pgt races PTKST (collected from South Africa), TTRTF (collected from Italy and Eritrea), TKKTF (collected from Italy), PCHSF (collected from Georgia) were used for phenotyping. In all cases, Sr26 wild type showed resistance, while the Sr26 mutants were susceptible to each pathotype (Table 2).

### Example 4—Exploring the Sr26 Homologs in Grass, Diploid Wheat, and Other Plants Genomes

According to BLAST best hits against IWGSC CS ref v1.0, the location of the closest homologs of the Sr26 candidate in Chinese Spring reference v1.0 is consistent with previous studies that this gene occurs in homoeologous chromosome group six. The inventors further extended the BLAST range of Sr26 to grass and diploid wheat genomes including T. monococcum, Aegilops tauschii, Ae. speltoides, Ae. sharoneenesis and T. urartu (FIG. 5).

### Example 5—Protein Structural Analysis of CNL Type Immune Receptors from Plants

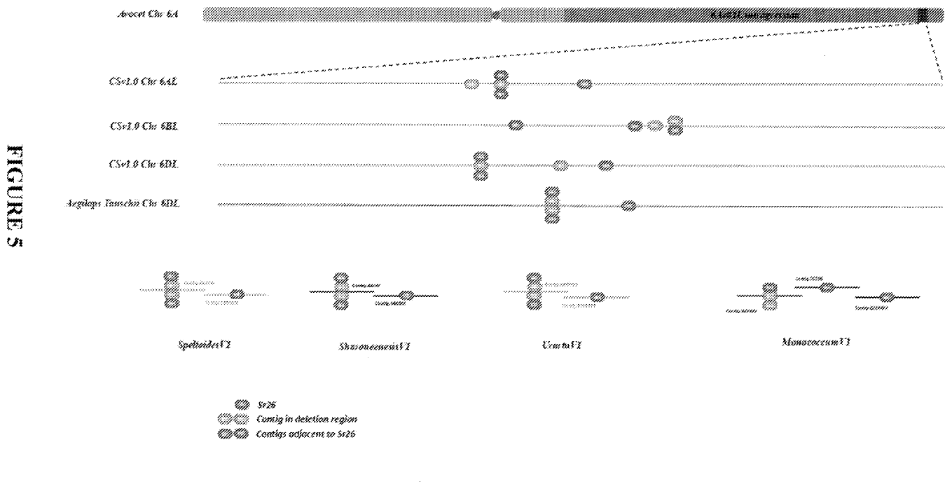

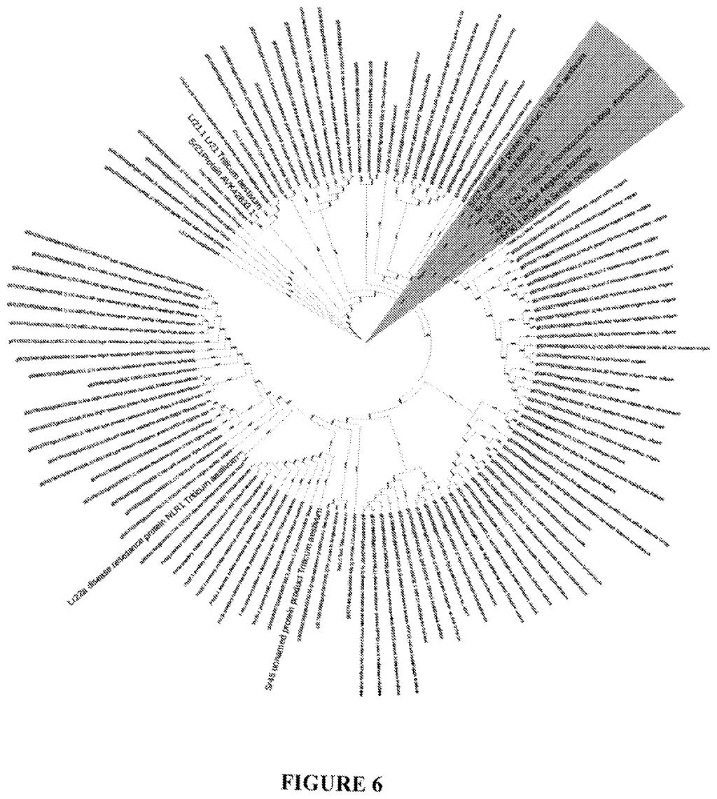

To determine the evolutionary distance and degree of diversity between Sr26 and other cloned CNL type R genes from plants at the protein sequence level, the inventors selected 124 CNL type R genes and performed a phylogenetic analysis (FIG. 6). The closest R gene to Sr26 from the selected group is the wheat stem rust resistance gene Sr13. The largest subgroup of wheat rust R genes includes Sr33, Sr50, Sr35 and Sr22 are clustered with the MLA R gene family. Wheat stem rust R gene Sr45 is grouped with the wheat powdery mildew R gene Pm3 and far apart from other wheat rust R genes. The wheat stem rust R gene Sr21 was closest to Pm2, Lr21, and the wheat nematode R genes Cre1 and Cre3.

TABLE 2
Phenotypic response score of Sr26 against various Pgt pathotypes.
a. Stem rust scores from six entries under glasshouse and field
conditions at both seedling and adult plant stages when
inoculated by PTKST. Equivalent results were
obtained in three independent experiments.

| | Adult plant stage | | | Seedling | |
|---|---|---|---|---|---|
| | | Stem | Leaf | leaf | |
| | Stem | infection | infection | infection | Field |
| Entries | Severity | type | type | type | score |
| Avocet + Lr46 | 20MR | 12− | ;1+ | 2− | 50MRMS |
| Avocet + Lr34 + | 20RMR | ;1 | ;1 | ; | 30RMR |
| Lr46 + Lr67 | | | | | |
| Kite (Sr26) | 20MR | 12− | ;12C | ;1− | 40MR |
| Sr26 mutant (12S) | 30MSS | 3+ | 2 + 3 | 3+ | 100S |
| Sr26 mutant (499S) | 30MSS | 3+ | 2 + 3 | 3+ | 100S |
| Line 37 | 30MSS | 3+ | 2 + 3 | 3+ | 100S |

b. Rust testing result of Sr26 against various Pgt pathotypes.

| Pgt pathotype | TTRTF | TKKTF | PCHSF | PTKST |
|---|---|---|---|---|
| Sr26 Wild-type | 1 | 1− | 1− | 2− |
| Sr26 Mutant | 3+ | 3+ | 2 | 3+ |

### Example 6—Cell Death Induction Tests for Sr50, Sr33, Sr35, Sr22, Sr45, Sr46 and Sr26 CC Domains

The CC domains of some CNL type R genes, including Sr33 and Sr50, have been shown to be able to trigger cell death in N. benthamiana. To test this function more generally for wheat CNLs, constructs expressing CC domains of Sr26, Sr22, Sr35, Sr45 and Sr46 were generated to perform transient expression assays and compared to constructs expressing Sr50 and Sr33 CC domains as controls. To define the minimal length of the CC domains to test, the protein sequence for all seven genes were aligned with Sr33 and Sr50. The CC domain from all genes were trimmed at the corresponding site of 160aa of Sr33, which has been previously demonstrated to be sufficient for CC domain cell death induction. Secondary structures have been reported previously to have an affect on the protein stability, therefore, sequences were trimmed appropriately to keep protein secondary structure units intact when determining the CC protein domain boundary. The predicted secondary structures of CC domains of each protein was performed using PSIPRED v3.3 program through PSIPRED server (http://bioinf.cs.ucl.ac.uk/psipred/) (FIG. 7a). Secondary structure predictions using PSIPRED v3.3 suggest all these CC domain fragments included the 4 a-helices of the known CC domain structure.

It was demonstrated that in addition to Sr33 and Sr50, the CC domains of Sr35 and Sr46 are also sufficient for cell death induction in planta. The CC domain of two new wheat stem rust resistance genes Sr35 and Sr46 are sufficient for triggering cell death in planta when fused with N-terminal YFP tag (FIG. 7b). In contrast, there was no cell death observed when the CC domain protein of Sr26, Sr45 and Sr22 in N. benthamiana was expressed fused with the same tag (FIG. 7b). However, Western Blot analyses (FIG. 7c) showed no detectable protein for Sr22 CC and low level of Sr26 and Sr45 CC domains, which may explain the lack of cell death induction (FIG. 7c). In some cases, fusion of tags can interfere with protein expression and function.

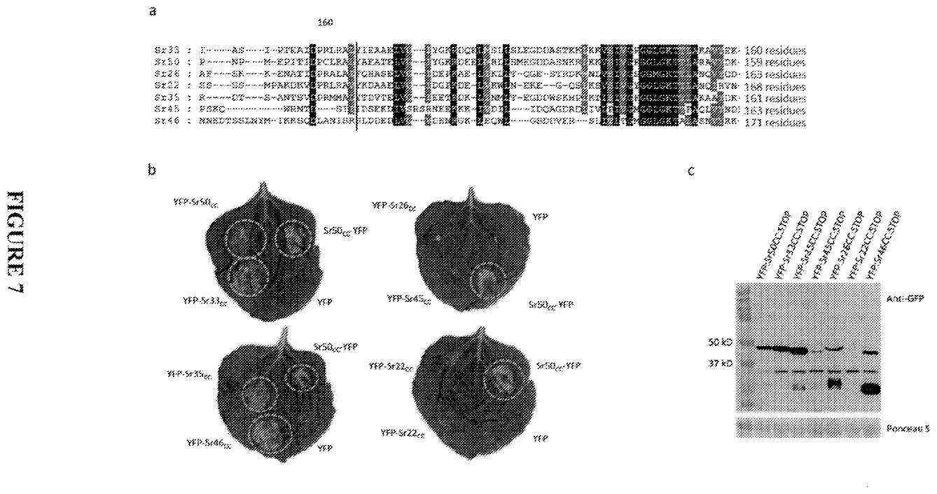

To avoid the potentially tag negative effect, the inventors tested the function of Sr22, Sr26 and Sr45 CC domains without tag. Interestingly, Sr22 CC domain was able to trigger cell death in N. benthamiana plants with no tag fused. However, in the case of Sr26 and Sr45 CC domains, without tag, no cell death induction was observed (FIG. 8).

### Example 7—Enhancement of Sr26 Resistance in Combination with APR Genes

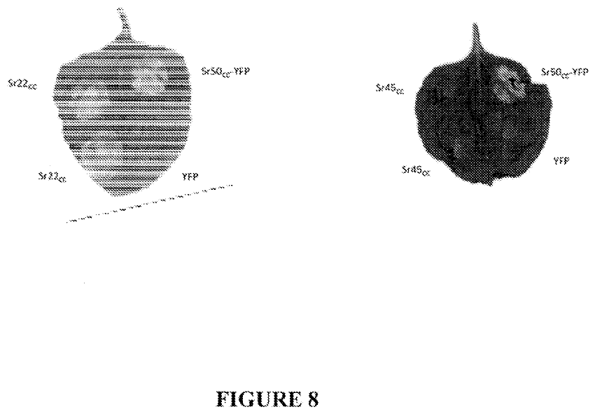

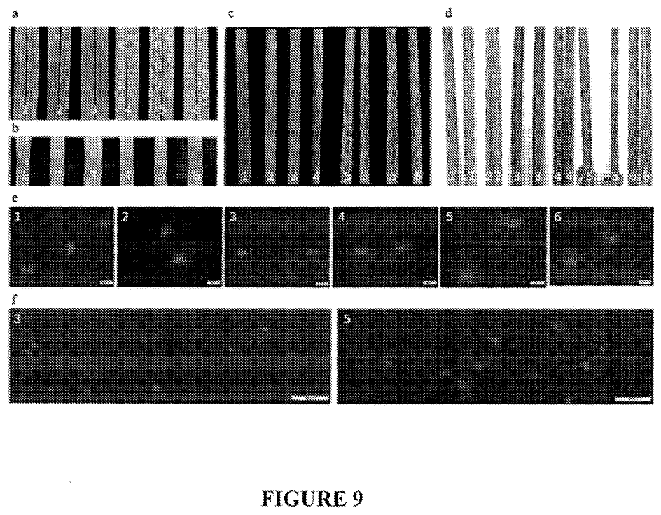

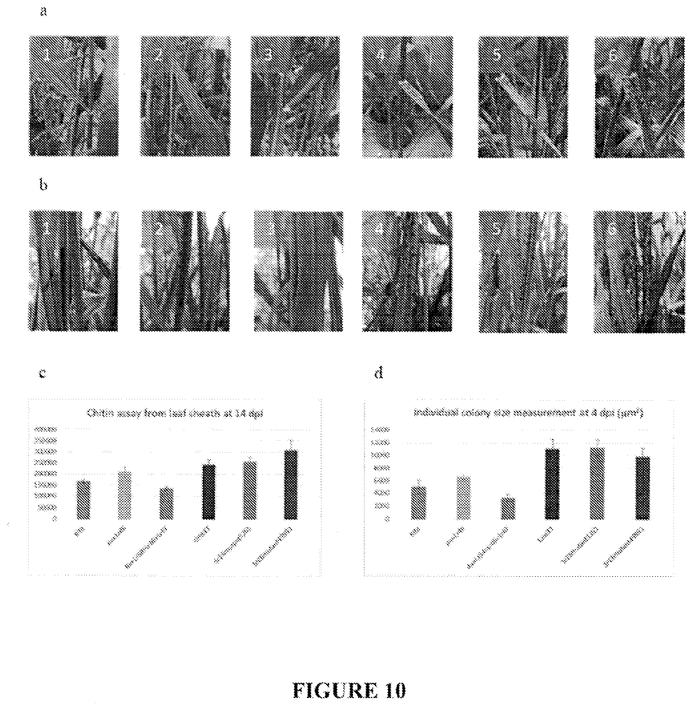

To enhance the deployment of Sr26 for achieving durable resistance, materials were generated that incorporated three pleiotropic genes Lr34/Yr18/Sr57, Lr46/Yr29/Sr58, and Lr67/Yr46/Sr56 in Avocet S background. The stem rust response of Kite (Sr26), Avocet (Sr26)+Lr46, Avocet(Sr26)+Lr34+Lr46+Lr67, together with Sr26 mutants 12S and 499S were compared at seedling and adult plant stages under both glasshouse and field conditions. Glasshouse experiments included phenotyping on seedling leaves (FIG. 9a, 9b; Table 2), adult plant stems (FIG. 9c, 9d; Table 2), adult plant flag leaves and leaf sheaths (FIG. 10a, 10b), whereas adult plant stems were rated in the field.

A chitin assay was also carried out on adult plant flag leaf sheaths (FIG. 10c) and measured the average individual colony size of PTKST at 4 dpi (FIG. 10d). In all cases, Avocet (Sr26)+Lr34+Lr46+Lr67 displayed stronger resistance compared to Kite (sr26) and Avocet (Sr26)+Lr46.

No additive effect or synergism between the three APR genes Lr34, Lr46 and Lr67 has been reported previously, but additive resistance has been found when either Lr34 or Sr26 are combined with other genes. Due to the infection type produced by Avocet(Sr26)+Lr46 was not as strong as the resistance given by three APRs involved Sr26 containing line, the indication seems to be this synergism is unlikely from the interaction between Lr46 and Sr26. To test this Fielder lines containing each single APR gene alone, each single APR with Sr26, two APR genes alone, two APR genes combined with Sr26, and three APR genes alone from a NIL population at both seedling and adult plant stages could be generated and grown. The generated plants could be infected with Puccinia graminis and the infection type classifed as described.

### Example 8—Discussion

Eight all stage stem rust resistance genes have been cloned that originate from T. monococcum (Sr21, Sr22, and Sr35) the A genome donor of hexaploid bread wheat, Ae. tauschii (Sr33 and Sr45) the D genome donor of hexaploid bread wheat, diploid rye (Sr50) and durum wheat (Sr13). Sr26 is the first wheat stem rust R gene identified from tall wheat grass (T. ponticum). Furthermore, Sr26 and Sr50 are the only two Sr genes from the tertiary gene pool of bread wheat. The present invention relates to Sr26, a locus with the broadest resistance to Pgt isolates worldwide, is a single gene encoding a CNL type immune receptor protein.

The inventors also demonstrated the Sr26 coding region together with its minimum native UTR regions (917 bp at 5′ and 263 bp at 3′ respectively) were sufficient to confer resistance. The addition of Sr22 and Sr33 regulatory elements fused with Sr26 native sets of promoter and terminator at TO was tested. All TO plants carrying the chimeric Sr26 gene fusions when tested with Pgt race 98-1,2,3,5,6 exhibited a resistance phenotype. Hatta et al. (2018) reported Sr45 gene function is not compromised when driven by Sr33 regulatory elements. It was observed here that Sr22 and Sr33 promoter and terminator did not negatively affect the expression of the Sr26 gene.

Conserved motifs of NB-ARC domain are among the most conserved residues in R proteins, suggesting to an important functional and structural role. This is found to be true in Sr26 from the mutated position of all mutants of Sr26 (mutant 128S is in RNBS-B, 70S/12S and 499S1 are in RNBS-D), and from Sr50 (mutant M13 in RNBS-B) and Sr33 (mutant E9 and E7 in P-loop, E6 in RNBS-B, and E8 in GLPL). It is further emphasized the importance of these domains in CNL gene function.

Nucleotide-binding leucine-rich repeat receptor (NLR) has long been known as immune receptors in plants. TIR-containing NLR (TNL) and CC-containing NLR (CNL) are two major classes of plant NLR that are defined based on the presence of either a TIR or CC domain at their N-terminus. The most, if not all, of NLRs in cereal are belong to CNL class. Although both TIR domains and CC domains were considered as predominant signaling elements of NLRs, they differ greatly from each other structurally and functionally (Ve et al., 2015). In comparison to the intensive studies of the TIR domain, the structure and function of how CC domain signaling downstream of effector perception are largely unknown. Previous studies have revealed diverse functions of CC domains including their ability to self-associate, induce cell death, and interact with other proteins as co-factors. For instance, the EDVID motif of the CC domain has been reported to play a role in regulating interaction with the NB domain of Rx (Hao et al., 2013). The CC domains of MLA10 and RPM1 also have the conserved EDVID motif, but only the MLA10 CC domain is able to signal cell death, while the CC domains of MLA10 and RPM1, but not Rx, can self-associate. RPM1 and Rx CC domains interact with co-factors required for pathogen pereption, while no such interactions are known for MLA10.

Maekawa et al. (2011) and Cesari et al. (2013) reported that MLA10, Sr33, and Sr50 CC domains are able to induce cell death in N. benthamiana. The present inventors have found that CC domains from Sr35, Sr46 and Sr22 expressed without a tag, induced cell death in planta. However, the CC domains from Sr26 and Sr45 did not induce cell death in planta. In the case of RX CC domain, induced cell death was not observed in model host plant although the construct used included. These results suggest that cell death induction by CC domains seems to be a common feature in stem rust resistance genes and further study is needed to further reveal the reason behind this divergency in R gene CC domain cell death induction.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

This application claims priority from AU 2018904568 filed 30 Nov. 2018, the entire contents of which are incorporated herein by reference.

All publications discussed and/or referenced herein are incorporated herein in their entirety.

Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed before the priority date of each claim of this application.

This invention was made with Government support under (965429) awarded by the National Science Foundation. The Government has certain rights in this invention.

### REFERENCES

Abdullah et al. (1986) Biotechnology 4:1087.
Ayliffe et al. (2011) Mol Plant Microbe. Interact. 24:1143-1155.
Bain et al. (2008) Proc S Afr Sug Technol Ass 81:508-512.
Barker et al. (1983) Plant Mol. Biol. 2: 235-350.
Begemann et al. (2017) Sci Rep. 7(1):11606.
Bender et al. (2016) Plant Disease 100, 1627-1633.
Bevan et al. (1983) Nucl. Acid Res. 11: 369-385.
Bhattacharya, S. (2017) Nature 542, 145.
Bulgarelli et al. (2010) PLoS One 5:e12599.
Cadwell and Joyce (1992) PCR Methods Appl. 2:28-33.
Capecchi (1980) Cell 22:479-488.
Cesari et al. (2013) Plant Cell 25, 1463-1481.
Chen et al. (2018) PLoS Genet 14, e1007287.
Clapp (1993) Clin. Perinatol. 20:155-168.
Coco et al. (2001) Nature Biotechnology 19:354-359.
Coco et al. (2002) Nature Biotechnology 20:1246-1250.
Comai et al. (2004) Plant J 37: 778-786.
Cooley et al. (2000) Plant Cell 12:663-676.
Curiel et al. (1992) Hum. Gen. Ther. 3:147-154.
Doudna and Charpentier (2014) Science 28:346(6213):1258096.
Dundas et al. (2015) Crop Science 55, 648-657.
Eggert et al. (2005) Chembiochem 6:1062-1067.
Eglitis et al. (1988) Biotechniques 6:608-614.
Enkhbayar et al. (2004) Proteins 54:394-403.
Fujimura et al. (1985) Plant Tissue Cultural Letters 2:74.
Garfinkel et al. (1983) Cell 27: 143-153.
Graham et al. (1973) Virology 54:536-539.
Grant et al. (1995) Plant Cell Rep. 15:254-258.
Greve (1983) J. Mol. Appl. Genet. 1:499-511.
Haft et al. (2005) Computational Biology, PLoS Comput Biol 1(6):e60.
Hao et al. (2013) J. Biol. Chem. 288:35868-35876.
Harayama (1998) Trends Biotechnol. 16:76-82.
Hatta et al. (2018) https://www.biorxiv.org/content/early/2018/07/23/374637
Hellinga (1997) Proc. Natl. Acad. Sci. 94:10015-10017.
Henikoff et al. (2004) Plant Physiol 135: 630-636.
Hinchee et al. (1988) Biotech. 6:915
Ishida et al. (2015) Agrobacterium Protocols, Vol 1, 3rd Edition 1223, 189-198.
Jézéquel et al. (2008) Biotechniques 45:523-532.
Jinek et al. (2012) Science 337:816-821.
Joshi (1987) Nucl. Acid Res. 15: 6643-6653.
Klee et al., (1985): Plant DNA Infectious Agents, Hohn and Schell, (editors), Springer-Verlag, New York, 179-203
Knott (1961). Canadian Journal of Plant Science 41, 109-123.
Krattinger et al. (2009) Science 323:1360-1363.
Liang et al. (2017) Nat Commun. 8:14261.
Liang et al. (2018) Plant Biotechnol J. 16:2053-2062.
Liang et al. (2019) Methods Mol Biol. 1917:327-335.
Langridge et al. (2001) Aust. J. Agric. Res. 52: 1043-1077.
Lemieux (2000) Current Genomics 1: 301-311.
Leung et al. (1989) Technique 1:11-15.
Lu et al. (1993) J. Exp. Med. 178: 2089-2096.
Lemieux (2000) Current Genomics 1: 301-311.
Leung et al. (1989) Technique 1:11-15.
Lu and Berry (2007) Protein Structure Design and Engineering, Handbook of Proteins 2: 1153-1157.
Luo et al. (2016) Plant Cell Rep 35(7):1439-1450.
Lupas et al. (1991) Science 252:1162-1164.
McHale et al. (2006) Genome Biology 7:212.
Ma et al. (2015) Molecular Plant 8: 1274-1284.
Maekawa et al. (2011) Cell Host Microbe 9:187-199.
Mago et al. (2002) Theor Appl Genet 104:1317-1324.
Mago et al. (2005) Theor Appl Genet. 112:41-50.
Makarova (2015) Nat. Rev. Microbiol. 13:722-736.
Medberry et al. (1992) Plant Cell 4: 185-192.
Meyers et al. (1999) Plant Journal 20:317-332.
Michelmore and Meyers (1998) Genome Res. 8:1113-1130.
Medberry et al. (1992) Plant Cell 4: 185-192.
Medberry et al. (1993) Plant J. 3: 619-626.
Moore et al. (2015) Genet 47:1494-1498.
Mundt (2018) Phytopathology 108:792-802.
Needleman and Wunsch (1970) J. Mol Biol. 45:443-453.
Ness et al. (2002) Nature Biotechnology 20:1251-1255.
Niedz et al. (1995) Plant Cell Reports 14: 403-406.
Ostermeier et al. (1999) Nature Biotechnology 17:1205-1209.
Ow et al. (1986) Science 234: 856-859.
Pan et al. (2000) J. Mol. Evol. 50:203-2013.
Park et al. (2009) Cereal Rust Report Season 2009. Vol. 7 Issue 2.
Periyannan et al. (2013) Science 341, 786-788.
Prasher et al. (1985) Biochem. Biophys. Res. Comm. 126: 1259-68.
Pretorius et al. (2000) Plant Disease 84, 203-203.
Pretorius et al. (2015) Phytoparasitica 43, 637-645.
Saintenac et al. (2013) Science 341, 783-786.
Sieber et al. (2001) Nature Biotechnology 19:456-460.
Sing et al. (2015) Phytopathology 105, 872-884.
Stalker et al. (1988) Science 242:419-423.
Stemmer (1994a) Proc. Natl. Acad. Sci. USA 91:10747-10751.
Stemmer (1994b) Nature 370(6488):389-391.
Steuernage et al. (2016) Nat Biotechnol 34, 652-655.
Slade and Knauf (2005) Transgenic Res. 14: 109-115.
Sun et al. (2016) Molecular Plant 9: 628-631.
Svitashev et al. (2016) Nat Commun. 7:13274.
Tameling et al. (2002) Plant Cell 14:2929-2939.
Traut (1994) Eur. J. Biochem. 222:9-19.
Thillet et al. (1988) J. Biol. Chem. 263:12500.
Toriyama et al. (1986) Theor. Appl. Genet. 205:34.
Ve et al. (2015) Apoptosis 20:251-261.
Volkov et al. (1999) Nucleic Acids Research 27:e18.
Wagner et al. (1992) Proc. Natl. Acad. Sci. USA 89:6099-6103.
Wang et al. (2011) New Phytologist. 191: 418-431.
Woo et al. (2015) Nat Biotechnol. 33:1162-1164.
Yu et al. (2017) Methods Mol Biol 1659, 207-213.
Zhang et al. (2017) Proc Natl Acad Sci USA 114, E9483-E9492.
Zhao et al. (1998) Nature Biotechnology 16:258-261.

1. A plant comprising an exogenous polynucleotide encoding a polypeptide which confers resistance to at least one strain of Puccinia graminis, wherein the polypeptide comprises amino acids having a sequence as provided in SEQ ID NO:1, a biologically active fragment thereof, or an amino acid sequence which is at least 70% identical to SEQ ID NO:1.

2. The plant of claim 1, wherein the polynucleotide is operably linked to a promoter capable of directing expression of the polynucleotide in a cell of the plant.

3. The plant of claim 1 or claim 2, wherein the Puccinia graminis is Puccinia graminis f. sp. tritici.

4. The plant according to any one of claims 1 to 3, wherein the strain is one or more or all of race TTRTF, PTKST, TKKTF, TKTTF and PCHSF of Puccinia graminis f. sp. tritici.

5. The plant according to any one of claims 1 to 4 which has enhanced resistance to at least one strain of Puccinia graminis when compared to an isogenic plant lacking the exogenous polynucleotide.

6. The plant according to any one of claims 1 to 5, wherein the polynucleotide comprises nucleotides having a sequence as provided in SEQ ID NO:2, a sequence which is at least 70% identical to SEQ ID NO:2, or a sequence which hybridizes to SEQ ID NO:2.

7. The plant according to any one of claims 1 to 6, wherein
i) the polypeptide comprises amino acids having a sequence which is at least 90% identical to SEQ ID NO:1, and/or
ii) the polynucleotide comprises a sequence which is at least 90% identical to SEQ ID NO:2.

8. The plant according to any one of claims 1 to 7, wherein the polypeptide comprises one, more or all of a coiled coil (CC) domain, an nucleotide binding (NB) domain and a leucine rich repeat (LRR) domain.

9. The plant according to any one of claims 1 to 8 which is a cereal plant such as a wheat plant.

10. The plant according to any one of claims 1 to 9 which comprises one or more further exogenous polynucleotides encoding another plant pathogen resistance polypeptide.

11. The plant according to any one of claims 1 to 10 which is homozygous for the exogenous polynucleotide.

12. The plant according to any one of claims 1 to 11 which is growing in a field.

13. A population of at least 100 plants according to any one of claims 1 to 12 growing in a field.

14. A process for identifying a polynucleotide encoding a polypeptide which confers resistance to at least one strain of Puccinia graminis comprising:
i) obtaining a polynucleotide operably linked to a promoter, the polynucleotide encoding a polypeptide comprising amino acids having a sequence as provided in SEQ ID NO:1, a biologically active fragment thereof, or an amino acid sequence which is at least 70% identical to SEQ ID NO:1,
ii) introducing the polynucleotide into a plant,
iii) determining whether the level of resistance to Puccinia graminis is modified relative to an isogenic plant lacking the polynucleotide, and
iv) optionally, selecting a polynucleotide which when expressed confers resistance to Puccinia graminis.

15. The process of claim 14, wherein one or more of the following apply,
a) the polynucleotide comprises nucleotides having a sequence as provided in SEQ ID NO:2, a sequence which is at least 82% identical to SEQ ID NO:2, or a sequence which hybridizes to SEQ ID NO:2,
b) the plant is a cereal plant such as a wheat plant,
c) the polypeptide is a plant polypeptide or mutant thereof, and
d) step ii) further comprises stably integrating the polynucleotide operably linked to a promoter into the genome of the plant.

16. The process of claim 14 or claim 15, wherein the strain is one or more or all of race TTRTF, PTKST, TKKTF, TKTTF and PCHSF of Puccinia graminis f. sp. tritici.

17. A substantially purified and/or recombinant polypeptide which confers resistance to at least one strain of Puccinia graminis, wherein the polypeptide comprises amino acids having a sequence as provided in SEQ ID NO:1, a biologically active fragment thereof, or an amino acid sequence which is at least 70% identical to SEQ ID NO:1.

18. The polypeptide of claim 17 which comprises amino acids having a sequence which is at least 80% identical, at least 90% identical, or at least 95% identical, to SEQ ID NO:1.

19. An isolated and/or exogenous polynucleotide comprising nucleotides having a sequence as provided in SEQ ID NO:2, a sequence which is at least 70% identical to SEQ ID NO:2, a sequence encoding a polypeptide of claim 17 or claim 18, or a sequence which hybridizes to SEQ ID NO:2.

20. A chimeric vector comprising the polynucleotide of claim 19.

21. The vector of claim 20, wherein the polynucleotide is operably linked to a promoter.

22. A recombinant cell comprising an exogenous polynucleotide of claim 19, and/or a vector of claim 20 or claim 21.

23. The cell of claim 22, wherein the cell is a cereal plant cell such as a wheat cell.

24. A method of producing the polypeptide claim 17 or claim 18, the method comprising expressing in a cell or cell free expression system the polynucleotide of claim 19.

25. A transgenic non-human organism, such as a transgenic plant, comprising an exogenous polynucleotide of claim 19, a vector of claim 20 or claim 21 and/or a recombinant cell of claim 22 or claim 23.

26. A method of producing the cell of claim 22 or claim 23, the method comprising the step of introducing the polynucleotide of claim 19, or a vector of claim 20 or claim 21, into a cell.

27. A method of producing a transgenic plant according to any one of claims 1 to 11, the method comprising the steps of
i) introducing a polynucleotide as defined in claim 19 and/or a vector of claim 21 into a cell of a plant,
ii) regenerating a transgenic plant from the cell, and
iii) optionally harvesting seed from the plant, and/or
iv) optionally producing one or more progeny plants from the transgenic plant, thereby producing the transgenic plant.

28. A method of producing a transgenic plant according to any one of claims 1 to 11, the method comprising the steps of
i) crossing two parental plants, wherein at least one plant is a transgenic plant according to any one of claims 1 to 11,
ii) screening one or more progeny plants from the cross for the presence or absence of the polynucleotide, and
iii) selecting a progeny plant which comprise the polynucleotide,
thereby producing the plant.

29. The method of claim 28, wherein at least one of the parental plants is a tetraploid or hexaploid wheat plant.

30. The method of claim 28 or claim 29, wherein step ii) comprises analysing a sample comprising DNA from the plant for the polynucleotide.

31. The method according to any one of claims 28 to 30, wherein step iii) comprises
i) selecting progeny plants which are homozygous for the polynucleotide, and/or
ii) analysing the plant or one or more progeny plants thereof for resistance to at least one strain of Puccinia graminis.

32. The method according to any one of claims 28 to 31, wherein the strain is one or more or all of race TTRTF, PTKST, TKKTF and PCHSF of Puccinia graminis f. sp. tritici.

33. The method according to any one of claims 28 to 32 which further comprises
iii) backcrossing the progeny of the cross of step i) with plants of the same genotype as a first parent plant which lacked a polynucleotide encoding a polypeptide which confers resistance to at least one strain of Puccinia graminis for a sufficient number of times to produce a plant with a majority of the genotype of the first parent but comprising the polynucleotide, and
iv) selecting a progeny plant which has resistance to the at least one strain of Puccinia graminis.

34. The method according to any one of claims 27 to 33, wherein the method further comprises the step of analysing the plant for at least one other genetic marker.

35. A plant produced using the method according to any one of claims 27 to 34.

36. Use of the polynucleotide of claim 19, or a vector of claim 20 or claim 21, to produce a recombinant cell and/or a transgenic plant.

37. The use of claim 36, wherein the transgenic plant has enhanced resistance to at least one strain of Puccinia graminis when compared to an isogenic plant lacking the exogenous polynucleotide and/or vector.

38. A method for identifying a plant comprising a polynucleotide encoding a polypeptide which confers resistance to at least one strain of Puccinia graminis, the method comprising the steps of
i) obtaining a nucleic acid sample from a plant, and
ii) screening the sample for the presence or absence of the polynucleotide, wherein the polynucleotide encodes a polypeptide of claim 17 or claim 18.

39. The method of claim 38, wherein the polynucleotide comprises nucleotides having a sequence as provided in SEQ ID NO:2, a sequence which is at least 70% identical to SEQ ID NO:2, or a sequence which hybridizes to SEQ ID NO:2.

40. The method of claim 38 or claim 39 which identifies a transgenic plant according to any one of claims 1 to 11.

41. The method of according to any one of claims 38 to 40 which further comprises producing a plant from a seed before step i).

42. A plant part of the plant according to any one of claim 1 to 11, 25 or 35.

43. The plant part of claim 42 which is a seed that comprises an exogenous polynucleotide which encodes a polypeptide which confers to at least one strain of Puccinia graminis.

44. A method of producing a plant part, the method comprising,
a) growing a plant according to any one of claim 1 to 11, 25 or 35, and
b) harvesting the plant part.

45. A method of producing flour, wholemeal, starch or other product obtained from seed, the method comprising;
a) obtaining seed according to claim 43, and
b) extracting the flour, wholemeal, starch or other product.

46. A product produced from a plant according to any one of claim 1 to 11, 25 or 35 and/or a plant part of claim 42 or claim 43.

47. The product of claim 46, wherein the part is a seed.

48. The product of claim 46 or claim 47, wherein the product is a food product or beverage product.

49. The product of claim 48, wherein
i) the food product is selected from the group consisting of: flour, starch, leavened or unleavened breads, pasta, noodles, animal fodder, animal feed, breakfast cereals, snack foods, cakes, malt, beer, pastries and foods containing flour-based sauces, or
ii) the beverage product is beer or malt.

50. The product of claim 46 or claim 47, wherein the product is a non-food product.

51. A method of preparing a food product of claim 48 or claim 49, the method comprising mixing seed, or flour, wholemeal or starch from the seed, with another food ingredient.

52. A method of preparing malt, comprising the step of germinating seed of claim 43.

53. Use of a plant according to any one of claim 1 to 11, 25 or 35, or part thereof, as animal feed, or to produce feed for animal consumption or food for human consumption.

54. A composition comprising one or more of a polypeptide of claim 17 or claim 18, a polynucleotide of claim 19, a vector of claim 20 or claim 21, or a recombinant cell of claim 22 or claim 23, and one or more acceptable carriers.

55. A method of identifying a compound that binds to a polypeptide comprising amino acids having a sequence as provided in SEQ ID NO:1, a biologically active fragment thereof, or an amino acid sequence which is at least 70% identical to SEQ ID NO:1, the method comprising:
i) contacting the polypeptide with a candidate compound, and
ii) determining whether the compound binds the polypeptide.
